(19) United States
(12) Patent Application Publication
(10) Pub. No.: US 2018/0346505 A1
(43) Pub. Date: Dec. 6, 2018

(54) MONONUCLEOTIDES HAVING A BIOREVERSIBLE DISULFIDE GROUP

(71) Applicant: SOLSTICE BIOLOGICS, LTD., Dublin, 2 (IE)

(72) Inventors: Curt W. BRADSHAW, San Diego, CA (US); Sukumar SAKAMURI, San Diego, CA (US); Dingguo LIU, San Diego, CA (US)

(21) Appl. No.: 15/534,022

(22) PCT Filed: Dec. 10, 2015
(86) PCT No.: PCT/US15/65026

§ 371 Date: Jun. 8, 2017

Related U.S. Application Data
(60) Provisional application No. 62/090,158, filed on Dec. 10, 2014.

(51) Int. Cl. C07H 19/10; A61P 31/14

(52) U.S. Cl. CPC C07H 19/10; C07B 2200/05; A61P 31/14

(57) ABSTRACT

The invention features a mononucleotide comprising a nucleobase bonded to a sugar having a 3′-carbon and a 5′-carbon, where the 5′-carbon is bonded to a phosphorus (V) atom of a phosphate group through an oxygen atom, the phosphorus (V) atom being bonded to (i) a disulfide bioreversible group through an oxygen atom; and (ii) (a) optionally substituted amino, optionally substituted alkoxy, optionally substituted aryloxy, or optionally substituted heteroaryloxy; or (b) the 3′-carbon through an oxygen atom. The invention also features methods of delivering the mononucleotide to a cell and methods of treating a subject having Hepatitis C.

### FIELD OF THE INVENTION

This invention relates to mononucleotides having a bioreversible disulfide group and methods of their use.

### BACKGROUND

The use of organophosphates for the treatment of diseases in humans has seen a recent increase with the approval of tenofovir, sofosbuvir, and cyclophosphamide. In vivo activity of some organophosphates requires the phosphate to be present with one or more negative charges. Inclusion of a negative charge in a drug compound, however, can decrease the pharmacological efficacy of the drug, because of the poor uptake of negatively charged molecules of certain size by cells. In one approach for the enhancement of the pharmacological efficacy of such drugs, the negatively charged oxygen atoms of a phosphate are masked as a phosphoester or as phosphamide. The challenge of this approach is in the requirement for rapid and reliable unmasking of the oxygen atoms of the organophosphate inside a cell and prevention of the premature extracellular unmasking. Attempts at the implementation of this approach mainly focused at the introduction of hydrolysable groups. These implementations, however, often present substantial disadvantages, such as the necessity for the co-location of an enzyme capable of unmasking the phosphate, toxicity of unmasking reaction by-products, and premature unmasking due to extracellular esterase of thioesterase activity.

Taken together, these issues present numerous challenges to drug discovery and development. An ideal prodrug and conjugation approach should be synthetically amenable, tolerate structural diversity, be universal among tissues, and consistent between species.

There remains a need for drug delivery approaches involving masking negative charge of organophosphates.

### SUMMARY OF THE INVENTION

In general, the present invention provides an approach for masking a mononucleotide.

In a first aspect, the invention provides a mononucleotide containing a nucleobase bonded to a sugar having a 3′-carbon and a 5′-carbon, wherein said 5′-carbon is bonded to a phosphorus (V) atom of a phosphate group through an oxygen atom, the phosphorus (V) atom being bonded to
(i) one and only one disulfide bioreversible group through an oxygen atom; and
(ii) (a) optionally substituted amino, optionally substituted C₁₋₆ alkoxy, optionally substituted C₆₋₁₄ aryloxy, or optionally substituted C₁₋₉ heteroaryloxy; or (b) the 3′-carbon through an oxygen atom.

In certain embodiments, the phosphate group can contain one and only one phosphorus (V) atom. In particular embodiments, the phosphorus (V) atom can be bonded to the 3′-carbon through an oxygen atom. In some embodiments, the phosphorus (V) atom can be bonded to optionally substituted amino, optionally substituted C₁₋₆ alkoxy, optionally substituted C₆₋₁₄ aryloxy, or optionally substituted C₁₋₉ heteroaryloxy. In particular, the phosphorus (V) atom can be bonded to optionally substituted amino or optionally substituted C₆₋₁₄ aryloxy. For example, the phosphorus (V) atom can be bonded to an optionally substituted amino.

In other embodiments, the disulfide bioreversible group can have a structure of formula (I):

G-S—S-(LinkA)-X (I),

in which

G is a functional cap group,

LinkA is a linker having a molecular weight greater than or equal to 28 Da, and

X is a bond to the oxygen atom of the phosphate group.

In yet other embodiments, the mononucleotide of the invention can be a compound of formula

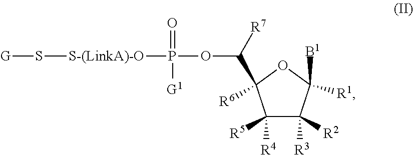

or a pharmaceutically acceptable salt or a phosphorus diastereomer thereof,

in which
G is a functional cap group;
LinkA is a linker;
B¹ is a nucleobase;
R¹ is H, azido, cyano, optionally substituted C₁₋₆ alkyl, optionally substituted C₂₋₆ alkenyl, or optionally substituted C₂₋₆ alkynyl;
each of R² and R³ is independently H, amino, azido, optionally substituted C₁₋₆ alkyl, optionally substituted C₁₋₆ heteroalkyl, optionally substituted C₂₋₆ alkenyl, optionally substituted C₂₋₆ alkynyl, halo, cyano, hydroxy, or optionally substituted C₁₋₆ alkoxy;
G¹ is optionally substituted amino, optionally substituted C₁₋₆ alkoxy, optionally substituted C₆₋₁₄ aryloxy, or optionally substituted C₁₋₉ heteroaryloxy, and R⁴ is hydroxy, optionally substituted C₁₋₆ alkoxy, optionally substituted amino, or azido, or G¹ and R⁴ combine to form —O—;
R⁵ is H, optionally substituted C₁₋₆ alkyl, optionally substituted C₁₋₆ heteroalkyl, optionally substituted C₂₋₆ alkenyl, optionally substituted C₂₋₆ alkynyl, or cyano;
R⁶ is H, azido, cyano, halo, optionally substituted C₁₋₆ alkyl, optionally substituted C₂₋₆ alkenyl, or optionally substituted C₂₋₆ alkynyl; and
R⁷ is H or optionally substituted C₁₋₆ alkyl.

In some embodiments of formula (I) or (II), G can be a blocking group, a delivery domain, or a dye.

In further embodiments, the mononucleotide of the invention can be a compound of formula (II), or a pharmaceutically acceptable salt or a phosphorus diastereomer thereof,

in which
G is optionally substituted C₃₋₁₀ alkyl, optionally substituted C₃₋₁₀ heteroalkyl, optionally substituted C₆₋₁₄ aryl, or optionally substituted C₁₋₆ heterocyclyl;
LinkA contains 1, 2, or 3 monomers independently selected from the group consisting of optionally substituted C₁₋₆ alkylene, optionally substituted C₁₋₆ heteroalkylene, optionally substituted C₆₋₁₄ arylene, optionally substituted C₁₋₆ heterocyclylene, optionally substituted aza, O, and S; wherein LinkA does not comprise two contiguous atoms selected from the group consisting of O and S, and wherein the monomer attached to the oxygen atom of the phosphate group is optionally substituted C₁₋₆ alkylene;
B¹ is a nucleobase;
R¹ is H, azido, cyano, optionally substituted C₁₋₆ alkyl, optionally substituted C₂₋₆ alkenyl, or optionally substituted C₂₋₆ alkynyl;
each of R² and R³ is independently H, amino, azido, optionally substituted C₁₋₆ alkyl, optionally substituted C₁₋₆ heteroalkyl, optionally substituted C₂₋₆ alkenyl, optionally substituted C₂₋₆ alkynyl, halo, cyano, hydroxy, or optionally substituted C₁₋₆ alkoxy;
G¹ is optionally substituted amino, optionally substituted C₁₋₆ alkoxy, optionally substituted C₆₋₁₄ aryloxy, or optionally substituted C₁₋₉ heteroaryloxy, and R⁴ is hydroxy, optionally substituted C₁₋₆ alkoxy, optionally substituted amino, or azido, or G¹ and R⁴ combine to form —O—; and
R⁵ is H, optionally substituted C₁₋₆ alkyl, optionally substituted C₁₋₆ heteroalkyl, optionally substituted C₂₋₆ alkenyl, optionally substituted C₂₋₆ alkynyl, or cyano;
R⁶ is H, azido, cyano, halo, optionally substituted C₁₋₆ alkyl, optionally substituted C₂₋₆ alkenyl, or optionally substituted C₂₋₆ alkynyl; and
R⁷ is H or optionally substituted C₁₋₆ alkyl.

In some embodiments of formula (II), R¹ can be H; R² can be optionally substituted C₁₋₆ alkyl; R³ can be hydroxy, optionally substituted C₁₋₆ alkoxy, or halo (e.g., R³ is halo); R⁵ can be H; R⁶ can be H; and/or R⁷ can be H or Me (e.g., R⁷ is H).

In other embodiments of formula (II), G¹ can be optionally substituted amino or optionally substituted C₆₋₁₄ aryloxy (e.g., G¹ is optionally substituted amino); and/or R⁴ can be hydroxy. Alternatively, G¹ and R⁴ can combine to form —O—.

In particular embodiments of formula (I) or (II), G can be a delivery domain (e.g., G is a delivery domain containing a targeting moiety, an endosomal escape moiety, or a cell penetrating peptide). In certain embodiments of formula (I) or (II), the targeting moiety can contain from 1 to 10 carbohydrates. Each carbohydrate can be independently GaINAc or mannose. The targeting moiety can alternatively be a lipid.

In further embodiments of formula (I) or (II), G can be a blocking group, such as optionally substituted C₃₋₁₀ alkyl, optionally substituted C₃₋₁₀ heteroalkyl, optionally substituted C₆₋₁₄ aryl, or optionally substituted C₁₋₉ heterocyclyl.

In some embodiments of formula (I) or (II), LinkA can contain 1, 2, or 3 monomers independently selected from the group consisting of optionally substituted C₁₋₆ alkylene, optionally substituted C₁₋₆ heteroalkylene, optionally substituted C₆₋₁₄ arylene, optionally substituted C₁₋₆ heterocyclylene, optionally substituted aza, O, and S; provided that LinkA does not contain two contiguous atoms selected from the group consisting of O and S, and wherein the monomer attached to the oxygen atom of said phosphate group is optionally substituted C₁₋₆ alkylene. For example, LinkA can contain 1, 2, or 3 monomers independently selected from the group consisting of optionally substituted C₁₋₆ alkylene, optionally substituted C₆₋₁₄ arylene, and O. In particular, LinkA can contain 1 or 2 monomers independently selected from the group consisting of optionally substituted C₁₋₆ alkylene and optionally substituted C₆₋₁₄ arylene.

In certain embodiments of formula (II), R³ is H, azido, optionally substituted C₁₋₆ alkyl, optionally substituted C₁₋₆ heteroalkyl, optionally substituted C₂₋₆ alkenyl, optionally substituted C₂₋₆ alkynyl, halo, cyano, hydroxy, or optionally substituted C₁₋₆ alkoxy; and/or R² is optionally substituted C₁₋₆ alkyl.

In further embodiments of the first aspect, the mononucleotide can be

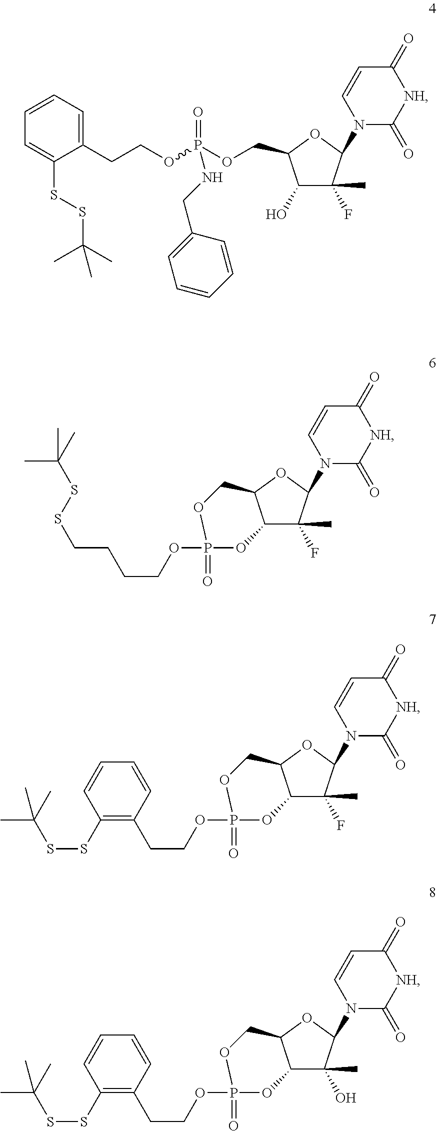

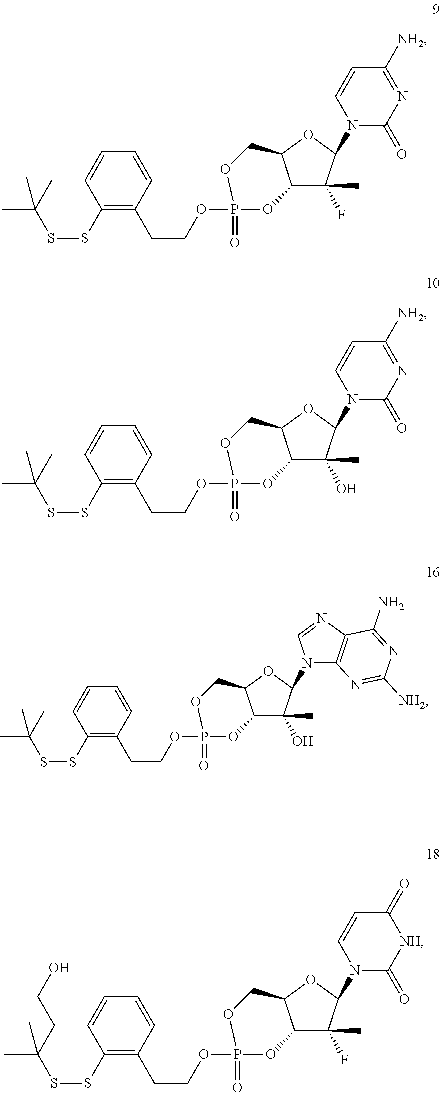

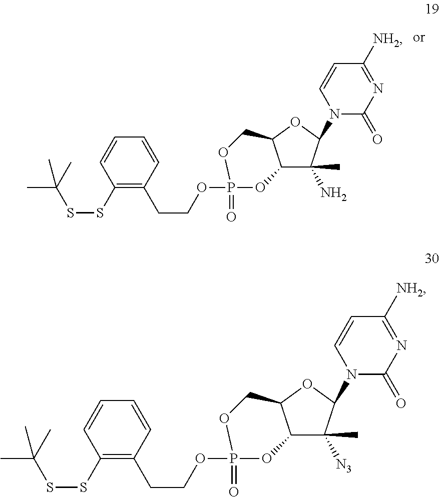

or a pharmaceutically acceptable salt or a phosphorus diastereomer thereof

The mononucleotide of the first aspect may also be in the form of an isotopically enriched composition, e.g., in a heavy isotope (e.g., ¹⁵N). For example, the nucleobase may include an isotopically enriched exocyclic amino group (e.g., cytosine).

In a second aspect, the invention provides a pharmaceutical composition containing a mononucleotide or the isotopically enriched composition of the first aspect. In certain embodiments of the second aspect, the pharmaceutical composition contains a pharmaceutically acceptable carrier.

In a third aspect, the invention provides a method of delivering a mononucleotide to a cell involving contacting the cell (e.g., a liver cell (hepatocyte)) with a mononucleotide or isotopically enriched composition of the first aspect.

In a fourth aspect, the invention provides a method of treating a subject (e.g., a human) having an RNA virus infection (e.g., hepatitis C) involving administering to the subject a mononucleotide or isotopically enriched composition of the first aspect. Alternatively, the pharmaceutical composition of the second aspect can be administered to the subject to treat an RNA virus infection (e.g., hepatitis C) in this subject.

### Definitions

The term “about,” as used herein, represents a value that is ±10% of the recited value.

The term “alkanoyl,” as used herein, represents a hydrogen or an alkyl group that is attached to the parent molecular group through a carbonyl group and is exemplified by formyl (i.e., a carboxyaldehyde group), acetyl, propionyl, butyryl, and iso-butyryl. Unsubstituted alkanoyl groups contain from 1 to 7 carbons. The alkanoyl group may be unsubstituted of substituted (e.g., optionally substituted C₁₋₇ alkanoyl) as described herein for alkyl group. The ending “-oyl” may be added to another group defined herein, e.g., aryl, cycloalkyl, and heterocyclyl, to define “aryloyl,” “cycloalkanoyl,” and “(heterocyclyl)oyl.” These groups represent a carbonyl group substituted by aryl, cycloalkyl, or heterocyclyl, respectively. Each of “aryloyl,” “cycloalkanoyl,” and “(heterocyclyl)oyl” may be unsubstituted or substituted as defined for “aryl,” “cycloalkyl,” or “heterocyclyl,” respectively.

The term “(C_{x1-y1} aryl)-C_{x2-y2}-alkyl,” as used herein, represents an aryl group of x1 to y1 carbon atoms attached to the parent molecular group through an alkylene group of x2 to y2 carbon atoms. Exemplary unsubstituted (C_{x1-y1} aryl)-C_{x2-y2}-alkyl groups are from 7 to 16 carbons. In some embodiments, the alkylene and the aryl each can be further substituted with 1, 2, 3, or 4 substituent groups as defined herein for the respective groups. Other groups followed by “alkyl” are defined in the same manner, where “alkyl” refers to a C₁₋₆ alkylene, unless otherwise noted, and the attached chemical structure is as defined herein.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkenylene,” as used herein, refers to a straight or branched chain alkenyl group with one hydrogen removed, thereby rendering this group divalent. Non-limiting examples of the alkenylene groups include ethen-1,1-diyl; ethen-1,2-diyl; prop-1-en-1,1-diyl, prop-2-en-1,1-diyl; prop-1-en-1,2-diyl, prop-1-en-1,3-diyl; prop-2-en-1,1-diyl; prop-2-en-1,2-diyl; but-1-en-1,1-diyl; but-1-en-1,2-diyl; but-1-en-1,3-diyl; but-1-en-1,4-diyl; but-2-en-1,1-diyl; but-2-en-1,2-diyl; but-2-en-1,3-diyl; but-2-en-1,4-diyl; but-2-en-2,3-diyl; but-3-en-1,1-diyl; but-3-en-1,2-diyl; but-3-en-1,3-diyl; but-3-en-2,3-diyl; buta-1,2-dien-1,1-diyl; buta-1,2-dien-1,3-diyl; buta-1,2-dien-1,4-diyl; buta-1,3-dien-1,1-diyl; buta-1,3-dien-1,2-diyl; buta-1,3-dien-1,3-diyl; buta-1,3-dien-1,4-diyl; buta-1,3-dien-2,3-diyl; buta-2,3-dien-1,1-diyl; and buta-2,3-dien-1,2-diyl. The alkenylene group may be unsubstituted or substituted (e.g., optionally substituted alkenylene) as described for alkyl.

The term “alkoxy,” as used herein, represents a chemical substituent of formula —OR, where R is a C₁₋₆ alkyl group, unless otherwise specified. In some embodiments, the alkyl group can be further substituted as defined herein. The term “alkoxy” can be combined with other terms defined herein, e.g., aryl, cycloalkyl, or heterocyclyl, to define an “aryl alkoxy,” “cycloalkyl alkoxy,” and “(heterocyclyl)alkoxy” groups. These groups represent an alkoxy that is substituted by aryl, cycloalkyl, or heterocyclyl, respectively. Each of “aryl alkoxy,” “cycloalkyl alkoxy,” and “(heterocyclyl)alkoxy” may be unsubstituted or substituted as defined herein for each individual portion.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four substituents independently selected from the group consisting of: amino; aryl; aryloxy; azido; cycloalkyl; cycloalkoxy; cycloalkenyl; cycloalkynyl; halo; heterocyclyl; (heterocyclyl)oxy; hydroxy; nitro; thiol; silyl; cyano; ═O; ═S; ═NR′, where R′ is H, alkyl, aryl, or heterocyclyl. Each of the substituents may itself be unsubstituted or, valency permitting, substituted with unsubstituted substituent(s) defined herein for each respective group.

The term “alkylamino,” as used herein, refers to a group having the formula —N(R^{N1})₂ or —NHR^{N1}, in which R^{N1} is alkyl, as defined herein. The alkyl portion of alkylamino can be optionally substituted as defined for alkyl. Each optional substituent on the substituted alkylamino may itself be unsubstituted or, valency permitting, substituted with unsubstituted subtituent(s) defined herein for each respective group.

The term “alkylene,” as used herein, refers to a saturated divalent, trivalent, or tetravalent hydrocarbon group derived from a straight or branched chain saturated hydrocarbon by the removal of at least two hydrogen atoms. Alkylene can be trivalent if bonded to one aza group that is not an optional substituent; alkylene can be trivalent or tetravalent if bonded to two aza groups that are not optional substituents. The valency of alkylene defined herein does not include the optional substituents. Non-limiting examples of the alkylene group include methylene, ethane-1,2-diyl, ethane-1,1-diyl, propane-1,3-diyl, propane-1,2-diyl, propane-1,1-diyl, propane-2,2-diyl, butane-1,4-diyl, butane-1,3-diyl, butane-1,2-diyl, butane-1,1-diyl, and butane-2,2-diyl, butane-2,3-diyl. The term “C_{x-y} alkylene” represents alkylene groups having between x and y carbons. Exemplary values for x are 1, 2, 3, 4, 5, and 6, and exemplary values for y are 2, 3, 4, 5, 6, 7, 8, 9, 10, 11, and 12. In some embodiments, alkylene can be further substituted with 1, 2, 3, or 4 substituent groups as defined herein for alkyl. Similarly, the suffix “ene,” when used in conjunction with a name of a radical defined herein, designates a divalent radical of the corresponding monovalent radical as defined herein. For example, alkenylene, arylene, aryl alkylene, cycloalkylene, cycloalkyl alkylene, cycloalkenylene, heteroarylene, heteroaryl alkylene, heterocyclylene, and heterocyclyl alkylene are divalent forms of alkenyl, alkynyl, aryl, aryl alkyl, cycloalkyl, cycloalkyl alkyl, cycloalkenyl, heteroaryl, heteroaryl alkyl, heterocyclyl, and heterocyclyl alkyl. For aryl alkylene, cycloalkyl alkylene, heteroaryl alkylene, and heterocyclyl alkylene, the two valences in the group may be located in the acyclic portion only or one in the cyclic portion and one in the acyclic portion.

The term “alkylsulfenyl,” as used herein, represents a group of formula —S-(alkyl). Alkylsulfenyl may be optionally substituted as defined for alkyl.

The term “alkylsulfinyl,” as used herein, represents a group of formula —S(O)-(alkyl). Alkylsulfinyl may be optionally substituted as defined for alkyl.

The term “alkylsulfonyl,” as used herein, represents a group of formula —S(O)₂-(alkyl). Alkylsulfonyl may be optionally substituted as defined for alkyl.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. The alkynyl groups may be unsubstituted or substituted (e.g., optionally substituted alkynyl) as defined for alkyl.

The term “amino,” as used herein, represents —N(R^{N1})₂ or —N(R^{N1})C(NR^{N1})N(R^{N1})₂ wherein each R^{N1} is independently H, —OH, —NO₂, —N(R^{N2})₂, —SO₂OR^{N2}, —SO₂R^{N2}, —SOR^{N2}, —COOR^{N2}, an N-protecting group, alkyl, alkenyl, alkynyl, alkoxy, aryl, aryloxy, cycloalkyl, cycloalkenyl, heteroalkyl, or heterocyclyl, and wherein each R^{N2} is independently H, alkyl, or aryl. In some embodiments, amino is —NH₂ or —NHR^{N1}, where R^{N1} is independently —OH, —SO₂OR^{N2}, —SO₂R^{N2}, —SOR^{N2}, —COOR^{N2}, alkyl, or aryl, and each R^{N2} can be alkyl or aryl. Each R^{N1} and R^{N2}, when present, may be independently unsubstituted or substituted as described herein (e.g., optionally substituted amino). In some embodiments, amino may be alkylamino. Each of the substituents may itself be unsubstituted or substituted with unsubstituted substituent(s) defined herein for each respective group. When amino is part of a functional cap group connected to the phosphorus (V) atom of the mononucleotide of the invention, any one of the substituents on the amino group may further include a delivery domain, a dye, or a blocking group.

The term “aryl,” as used herein, represents a mono-, bicyclic, or multicyclic carbocyclic ring system having one or two aromatic rings. An aryl group may include from 6 to 14 carbon atoms (e.g., from 6 to 10 carbon atoms). All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be optionally substituted with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; and cyano. Each of the substituents may itself be unsubstituted or substituted with unsubstituted substituent(s) defined herein for each respective group.

The term “aryloxy,” as used herein, represents a chemical substituent of formula —OR, where R is an aryl group, unless otherwise specified. In some embodiments, the aryl group can be further substituted as defined herein.

The term “aza,” as used herein, represents a divalent —N(R^{N1})— group or a trivalent —N═ group. The aza group may be unsubstituted, where R^{N1} is H or absent, or substituted, where R^{N1} is as defined for “amino,” except R^{N1} is not H. Two aza groups may be connected to form “diaza.”

The term “azido,” as used herein, represents an —N₃ group.

The term “blocking group,” as used herein, refers to a chemical group that is inert under physiological conditions and has at least one carbon atom. The at least one carbon atom of the blocking group is bonded to —S—S— of the disulfide bioreversible group.

The term “bulky group,” as used herein, represents any substituent or a group of substituents as defined herein, in which the radical of the bulky group bears one hydrogen atom or fewer if the radical is sp³-hybridized carbon, or bears no hydrogen atoms if the radical is sp²-hybridized carbon. The radical is not sp-hybridized carbon. The bulky group bonds to another group only through a carbon atom. For example, the statements “bulky group bonded to the disulfide linkage,” “bulky group attached to the disulfide linkage,” and “bulky group linked to the disulfide linkage” indicate that the bulky group is bonded to the disulfide linkage through a carbon radical.

The term “carbocyclic,” as used herein, represents an optionally substituted C₃₋₁₂ monocyclic, bicyclic, or tricyclic structure in which the rings, which may be aromatic or non-aromatic, are formed by carbon atoms. Carbocyclic structures include cycloalkyl, cycloalkenyl, cycloalkynyl, and certain aryl groups.

The term “carbohydrate,” as used herein, represents a compound which comprises one or more monosaccharide units having at least 5 carbon atoms (which may be linear, branched or cyclic) with an oxygen, nitrogen or sulfur atom bonded to each carbon atom. The term “carbohydrate” therefore encompasses monosaccharides, disaccharides, trisaccharides, tetrasaccharides, oligosaccharides, and polysaccharides. Representative carbohydrates include the sugars (mono-, di-, tri- and oligosaccharides containing from about 4-9 monosaccharide units), and polysaccharides such as starches, glycogen, cellulose, and polysaccharide gums. Specific monosaccharides include C₅₋₆ sugars; di- and trisaccharides include sugars having two or three monosaccharide units (e.g., C₅₋₆ sugars).

The term “carbonyl,” as used herein, represents a C(O) group. Examples of functional groups which comprise a “carbonyl” include esters, ketones, aldehydes, anhydrides, acyl chlorides, amides, carboxylic acids, and carboxylates.

The term “cyano,” as used herein, represents —CN group.

The term “cycloalkenyl,” as used herein, refers to a non-aromatic carbocyclic group having at least one double bond in the ring and from three to ten carbons (e.g., a C₃-C₁₀ cycloalkenyl), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenyl alkyl,” as used herein, represents an alkyl group substituted with a cycloalkenyl group, each as defined herein. The cycloalkenyl and alkyl portions may be substituted as the individual groups defined herein.

The term “cycloalkenylene,” as used herein, refers to a divalent carbocyclic non-aromatic group having at least one double bond in the ring and from three to ten carbons (e.g., C₃-C₀ cycloalkenylene), unless otherwise specified. Non-limiting examples of the cycloalkenylene include cycloprop-1-en-1,2-diyl; cycloprop-2-en-1,1-diyl; cycloprop-2-en-1,2-diyl; cyclobut-1-en-1,2-diyl; cyclobut-1-en-1,3-diyl; cyclobut-1-en-1,4-diyl; cyclobut-2-en-1,1-diyl; cyclobut-2-en-1,4-diyl; cyclopent-1-en-1,2-diyl; cyclopent-1-en-1,3-diyl; cyclopent-1-en-1,4-diyl; cyclopent-1-en-1,5-diyl; cyclopent-2-en-1,1-diyl; cyclopent-2-en-1,4-diyl; cyclopent-2-en-1,5-diyl; cyclopent-3-en-1,1-diyl;cyclopent-1,3-dien-1,2-diyl; cyclopent-1,3-dien-1,3-diyl; cyclopent-1,3-dien-1,4-diyl; cyclopent-1,3-dien-1,5-diyl; cyclopent-1,3-dien-5,5-diyl; norbornadien-1,2-diyl; norbornadien-1,3-diyl; norbornadien-1,4-diyl; norbornadien-1,7-diyl; norbornadien-2,3-diyl; norbornadien-2,5-diyl; norbornadien-2,6-diyl; norbornadien-2,7-diyl; and norbornadien-7,7-diyl. The cycloalkenylene may be unsubstituted or substituted (e.g., optionally substituted cycloalkenylene) as defined for cycloalkyl.

The term “cycloalkoxy,” as used herein, represents a chemical substituent of formula —OR, where R is cycloalkyl group, unless otherwise specified. In some embodiments, the cycloalkyl group can be further substituted as defined herein.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1]heptyl, 2-bicyclo[2.2.1]heptyl, 5-bicyclo[2.2.1]heptyl, 7-bicyclo[2.2.1]heptyl, and decalinyl. The cycloalkyl group may be unsubstituted or substituted (e.g., optionally substituted cycloalkyl) with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “cycloalkyl alkyl,” as used herein, represents an alkyl group substituted with a cycloalkyl group, each as defined herein. The cycloalkyl and alkyl portions may be substituted as the individual groups described herein.

The term “cycloalkynyl,” as used herein, refers to a monovalent carbocyclic group having one or two non-contiguous carbon-carbon triple bonds and having from eight to ten carbons (e.g., a C₈-C₁₀ cycloalkynyl), unless otherwise specified. Non-limiting examples of cycloalkynyl include cyclooctynyl, cyclononynyl, cyclodecynyl, and cyclodecadiynyl. The cycloalkynyl group may be unsubstituted or substituted (e.g., optionally substituted cycloalkynyl) as defined for cycloalkyl.

The term “cycloalkynyl alkyl,” as used herein, represents an alkyl group substituted with a cycloalkynyl group, each as defined herein. The cycloalkynyl and alkyl portions may be substituted as the individual groups described herein.

The term “disulfide bioreversible group,” as used herein, represents a moiety including a disulfide group (—S—S—). The disulfide group can be actively cleaved intracellularly, e.g., via the action of one or more intracellular enzymes (e.g., an intracellar reductase) or passively cleaved intracellularly, e.g., by exposing the group to the intracellular environment or a condition present in the cell (e.g., reductive or oxidative environment, or reaction with intracellular species, such as glutathione).

The term “endosomal escape moiety,” as used herein, represents a moiety which enhances the release of endosomal contents or allows for the escape of a molecule from an intracellular compartment, such as an endosome.

The term “halo,” as used herein, represents a halogen selected from bromine, chlorine, iodine, and fluorine.

The term “heteroalkyl,” as used herein refers to an alkyl, alkenyl, or alkynyl group interrupted once by one or two heteroatoms; twice, each time, independently, by one or two heteroatoms; three times, each time, independently, by one or two heteroatoms; or four times, each time, independently, by one or two heteroatoms. Each heteroatom is, independently, O, N, or S. In some embodiments, the heteroatom is O or N. None of the heteroalkyl groups includes two contiguous oxygen or sulfur atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteratom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N-protecting group, alkyl, alkenyl, alkynyl, aryl, cycloalkyl, cycloalkenyl, cycloalkynyl, heterocyclyl, or cyano, where each R^{N2} is independently H, alkyl, cycloalkyl, cycloalkenyl, cycloalkynyl, aryl, or heterocyclyl, and each R^{N3} is independently alkyl, cycloalkyl, cycloalkenyl, cycloalkynyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group.

The term “heteroaryloxy,” as used herein, refers to a structure —OR, in which R is heteroaryl. Heteroaryloxy can be optionally substituted as defined for heterocyclyl.

The term “heterocyclyl,” as used herein, represents a monocyclic, bicyclic, tricyclic, or tetracyclic ring system having fused or bridging 5-, 6-, or 7-membered rings, unless otherwise specified, containing one, two, three, or four heteroatoms independently selected from the group consisting of nitrogen, oxygen, and sulfur. Heterocyclyl can be aromatic or non-aromatic. Non-aromatic 5-membered heterocyclyl has zero or one double bonds, and non-aromatic 6- and 7-membered heterocyclyl groups have zero to two double bonds. Certain heterocyclyl groups include from 2 to 12 carbon atoms. Other such groups may include up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, etc. If the heterocyclic ring system has at least one aromatic resonance structure or at least one aromatic tautomer, such structure is an aromatic heterocyclyl (i.e., heteroaryl). Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be optionally substituted with one, two, three, four or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “heterocyclyl alkyl,” as used herein, represents an alkyl group substituted with a heterocyclyl group, each as defined herein. The heterocyclyl and alkyl portions may be substituted as the individual groups described herein.

The term “(heterocyclyl)oxy,” as used herein, represents a chemical substituent of formula —OR, where R is a heterocyclyl group, unless otherwise specified. In some embodiments, the heterocyclyl group can be further substituted as defined herein.

The terms “hydroxyl” and “hydroxy,” as used interchangeably herein, represent an —OH group.

The term “isotopically enriched,” as used herein, refers to a composition including an isotope, e.g., ¹⁵N, in the mononucleotide in an abundance greater than found naturally. Typically and depending on the isotope, compositions enriched in a particular isotope may have an isotopic enrichment factor of at least 5, at least 10, at least 50, at least 500, at least 2000, at least 3000, at least 6000, or at least 6600. When the composition is isotopically enriched, the compound is preferably enriched in a heavy isotope, i.e., an isotope of the specified element having an isotopic mass greater than the isotopic mass of the naturally most abundant isotope of the specified element.

The term “isotopic enrichment factor,” as used herein, refers to the mole percentage of the specified isotope in the specified composition relative to the naturally occurring abundance of that isotope.

The term “mononucleoside,” as used herein, represents a sugar-nucleobase compound. Non-limiting examples of mononucleosides are found in the following compounds: sofosbuvir, VX-135, IDX21437, IDX20963, ACH3422, mericitabine, valopicitabine, balapiravir, MK0608, GS-6620, IDX184, IDX19368, INX189, PSI938, PSI661, RS-1389, and those disclosed in WO 2005/003147, WO 2009/067409, and WO 2010/108140, the mononucleosides of which are incorporated herein.

The term “mononucleotide,” as used herein, refers to a mononucleoside, the 5′-carbon of which is bonded to a phosphate group.

The term “nitro,” as used herein, represents an —NO₂ group.

The term “nucleobase,” as used herein, represents a nitrogen-containing heterocyclic ring system found at the 1′ position of the sugar of a nucleoside. Nucleobases can be unmodified or modified. As used herein, “unmodified” or “natural” nucleobases include purine bases (e.g., adenine (A) or guanine (G)) or pyrimidine bases (e.g., thymine (T), cytosine (C), or uracil (U)). A modified nucleobase can be a protected version of the purine or pyrimidine base, in which one or more oxygen and/or nitrogen atoms is protected with an appropriate protecting group or is present as a prodrug moiety. A modified nucleobase can be an O- or N-alkylated version of the purine or pyrimidine base. Modified nucleobases include aza- and deaza- modifications of adenine, guanine, thymine, cytosine, and uracil. In particular, aza modifications include substitution of one or more carbon atoms within the purine or pyrimidine base with a nitrogen atom. Deaza-modifications include substitution of one or more nitrogen atoms within the purine or pyrimidine base with a carbon atom. In a non-limiting example, a purine base can be modified to include aza- and deaza- modifications, thereby forming a pyrrolo[2,1-f] [1,2,4]triazine. Additionally or alternatively, modifications of the purine or pyrimidine base may include the alteration of the unsaturation degree of the base to higher or lower than that of the initial base. Additionally or alternatively, the pyrimidine or purine base may be rendered unsubstituted or substituted with substituents defined for aryl or heterocyclyl, as appropriate.

The term “oxo,” as used herein, represents a divalent oxygen atom (e.g., the structure of oxo may be shown as ═O).

The term “Ph,” as used herein, represents phenyl.

The term “phosphate group,” as used herein, refers to a molecular fragment having a phosphorus (V) atom bonded to 2, 3, or 4 oxygen atoms, optionally one sulfur atom, and optionally one nitrogen atom, provided that the total number of atoms bonded to the phosphorus (V) atom is equal to 4.

The term “phosphorus (V) atom,” as used herein, refers to a phosphorus atom in the formal oxidation state (V). Within compounds of the invention, a phosphorus (V) atom has five valencies, two of which are occupied by ═O or ═S, one or two of the remaining three valencies is bonded to a mononucleoside, and one valency is bonded to a disulfide bioreversible group. The phosphorus (V) atom may be a part of a phosphate group. One or two oxygen atom(s) of the phosphate group is/are a part of a mononucleoside.

The term “physiological conditions,” as used herein, refer to the conditions that may exist inside a living mammalian cell (e.g., a liver cell). The physiological conditions include temperatures from about 34° C. to about 43° C. (e.g., from about 35° C. to about 42° C.) and aqueous pH from about 6 to about 8 (e.g., from about 6.5 to about 7.8).

The term “protecting group,” as used herein, represents a group intended to protect a hydroxy, an amino, or a carbonyl from participating in one or more undesirable reactions during chemical synthesis. The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Greene, “Protective Groups in Organic Synthesis,” 3^{rd} Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-n itrophenoxyacetyl, a-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-n itrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyI)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, triphenylmethyl, benzyloxymethyl, and the like and silyl groups such as trimethylsilyl, and the like. Useful N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “silyl,” as used herein, represents a group having the structure —SiR′₃, in which each R′ is independently selected from the group consisting of H, alkyl, aryl, cycloalkyl, cycloalkenyl, cycloalkynyl, heteroalkyl, and heterocyclyl. The silyl group may be unsubstituted or substituted (e.g., optionally substituted silyl). When silyl is substituted, at least one R′ includes at least one unsubstituted or substituted substituent selected from those defined for the group in question. In some embodiments, each R′ is independently unsubstituted alkyl or unsubstituted aryl.

The term “subject,” as used herein, represents a human or non-human animal (e.g., a mammal). In some embodiments, the subject may be suffering from hepatitis C, as determined by a qualified professional (e.g., a doctor or a nurse practitioner) with or without known in the art laboratory test(s) of sample(s) from the subject.

The term “sulfide” as used herein, represents a divalent —S— or ═S group. Disulfide is —S—S—.

The term “targeting moiety,” as used herein, represents any moiety that specifically, covalently or non-covalently binds to a receptor (e.g., a cell surface receptor) or other receptive moiety associated with a given target cell population.

The term “therapeutically effective dose,” as used herein, represents the quantity of the mononucleotide of the invention necessary to ameliorate, treat, or at least partially arrest the symptoms of a disease or disorder (e.g., hepatitis C). Amounts effective for this use depend on the severity of the disease and the weight and general state of the subject. Typically, dosages used in vitro may provide useful guidance in the amounts useful for in vivo administration of the pharmaceutical composition, and animal models may be used to determine effective dosages for treatment of a particular disease (e.g., hepatitis C).

The term “thiocarbonyl,” as used herein, represents a C(═S) group.

The term “thiol,” as used herein, represents an —SH group.

The term “treating” as used in reference to a disorder in a subject, is intended to refer to reducing at least one symptom of the disorder by administrating a therapeutic (e.g., the mononucleotide of the invention) to the subject.

As used herein and in the appended claims, the singular forms “a,” “and,” and “the” include plural referents unless the context clearly dictates otherwise. Thus, for example, reference to “a targeting moiety” includes a plurality of such targeting moieties, and reference to “the cell” includes reference to one or more cells known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

“Comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

For any term present in the art which is identical to any term expressly defined in this disclosure, the term's definition presented in this disclosure will control in all respects.

Each position in the compounds of the invention may include elements in their natural isotopic abundance. Alternatively, one or more positions in the compound of the invention may include an element enriched in a naturally occurring or a synthetic isotope. For example, one or more positions of the compound of the invention including hydrogen may be enriched with, e.g., deuterium or tritium. In some embodiments, one or more positions of the compound of the invention including carbon may be enriched with, e.g., ¹⁴C or ¹³C. In other embodiments, one or more positions of the compound of the invention including nitrogen may be enriched with, e.g., ¹⁵N. In certain embodiments, one or more positions of the compound of the invention including oxygen may be enriched with, e.g., ¹⁸O, ¹⁷O, or ¹⁵O. In particular embodiments, one or more positions of the compound of the invention including fluorine may be enriched with, e.g., ¹⁸F. In other embodiments, one or more positions of the compound of the invention including carbon may be enriched with, e.g., ³²S, ³³S, ³⁴S, ³⁵S, or ³⁶S. In yet other embodiments, one or more positions of the compound of the invention including chlorine may be enriched with, e.g., ³⁵Cl, ³⁶Cl, or ³⁷Cl.

### BRIEF DESCRIPTION OF THE DRAWINGS

FIG. 1 shows a structure of the hepatitis C viral (HCV) replicons. The HCV replicons contain the 5′ end of HCV (with HCV Internal Ribosome Entry Site, IRES and the first few amino acids of the HCV core protein) which drives the production of HCV core-neomycin phosphotransferase (Neo^{R}) fusion protein. The EMCR IRES element (E-1) controls the translation of the HCV structural proteins NS3-NS5. The NS3 protein cleaves the HCV polyprotein to release the mature NS3, NS4A, NS4B, NSSA and NS5B proteins that are required for HCV replication. At the 3′ end of the replicon is the authentic 3′NTR of HCV.

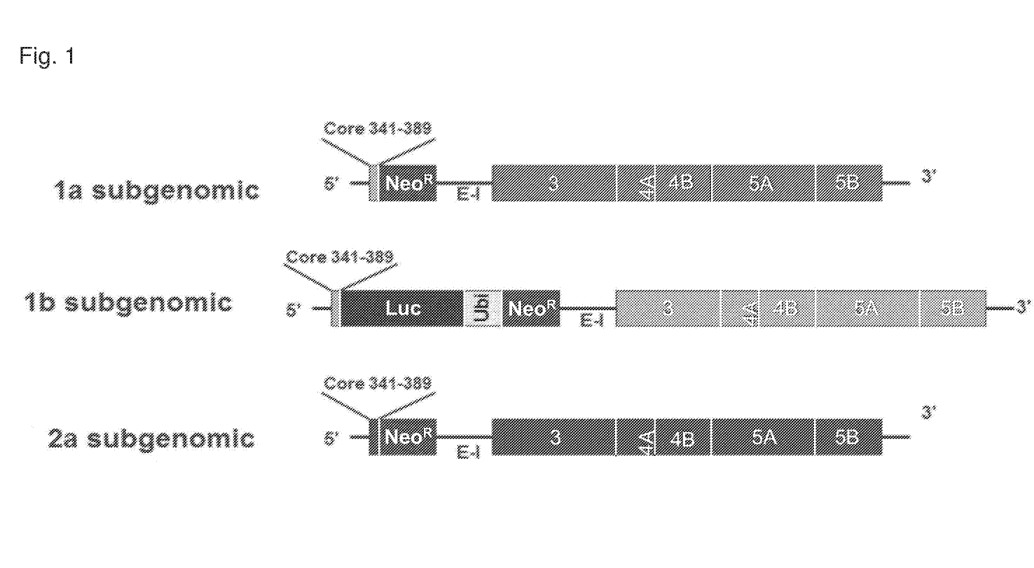

FIG. 2 is a chart showing mouse serum stability of nucleoside phosphoesters.

FIG. 3 is a chart showing rat serum stability of nucleoside phosphoesters.

FIG. 4 is a chart showing human serum stability of nucleoside phosphoesters.

FIG. 5 is a chart showing intracellular levels of active nucleoside triphosphates in vitro in Huh7 cells.

FIG. 6 is a chart showing intracellular levels of active nucleoside triphosphates in vitro in primary human hepatocytes.

FIG. 7 is a chart showing intracellular levels of active nucleoside triphosphates in rat liver homogenate isolated from rats dosed intravenously with nucleoside phosphoesters.

FIG. 8 is a chart showing intracellular levels of active nucleoside triphosphates in rat liver homogenate isolated from rats dosed orally with nucleoside phosphoesters.

### DETAILED DESCRIPTION

In general, the present invention relates to an approach for masking a phosphate in mononucleotides. In the present approach, one of the negative charges of a phosphate group in a mononucleotide is masked with a disulfide bioreversible group. Without being bound by a theory, the disulfide bioreversible group undergoes rapid sulfur-sulfur bond cleavage inside a cell, as an intracellular medium can be more reducing than an extracellular medium. The reliance on the intracellular reduction can overcome the challenge of premature extracellular unmasking of a phosphate.

Mononucleotides of the invention possess enhanced stability in serum and gastrointestinal fluids relative to other mononucleotide prodrugs. Further, mononucleotides of the invention exhibit greater potency relative to other mononucleotide prodrugs.

The present invention features a mononucleotide containing a nucleobase bonded to a sugar having a 3′-carbon and a 5′-carbon, where the 5′-carbon is bonded to a phosphorus (V) atom of a phosphate group through an oxygen atom, the phosphorus (V) atom being bonded to (i) a disulfide bioreversible group through an oxygen atom, and (ii) (a) optionally substituted amino, optionally substituted alkoxy, or optionally substituted aryloxy, or (b) the 3′-carbon through an oxygen atom.

### Disulfide Bioreversible Group

Disulfide bioreversible groups included in the mononucleotides of the invention can contain a bulky group proximal to —S—S—. The inclusion of the bulky group proximal to —S—S— can facilitate the preparation of the mononucleotides of the invention as described herein without significant losses of the material due to the sulfur-sulfur bond cleavage.

The sulfur atoms of the disulfide bioreversible group can be separated from the phosphate group by at least 2 contiguous atoms. In some embodiments, —S—S— of the disulfide bioreversible group can be separated from the phosphate group by at least 3 contiguous atoms. Without being bound by a theory, the separation between the disulfide group and the phosphate group allows for extrusion and cyclization of a portion of the atomic chain (e.g., —S-(LinkA)-) connected to the phosphate group with a concomitant release of the mononucleotide having an unmasked or partially unmasked phosphate group upon cleavage of the sulfur-sulfur bond inside a living cell.

The disulfide bioreversible group may have a structure of formula (I):

G-S—S-(LinkA)-X (I),

where

G is a first functional cap group,

LinkA is a linker having a molecular weight greater than or equal to 28 Da, and

X is a bond to the oxygen atom of a phosphate group.

LinkA

LinkA is a linker that includes an sp³-hybridized carbon atom bonded to —O— in formula (I) or (II). This structural feature permits the detachment of LinkA from the oxygen atom connected to the phosphorus (V) atom of formula (I) or (II). LinkA does not contain two contiguous atoms selected from O and S. LinkA may have a molecular weight greater than or equal to 28 Da (e.g., greater than or equal to 56 Da). LinkA may have a molecular weight less than or equal to 1000 Da (e.g., less than or equal to 300 Da). For example, the molecular weight of LinkA may be from 28 Da to 1000 Da (e.g., from 28 Da to 300 Da or from 56 Da to 300 Da). LinkA may include 1, 2, or 3 monomers linked together in a chain connecting G-S—S— and —O— in formula (I) or (II). Each of these monomers is independently optionally substituted C₁₋₆ alkylene, optionally substituted C₁₋₆ heteroalkylene, optionally substituted C₆₋₁₄ arylene, optionally substituted C₁₋₉ heterocyclylene, optionally substituted aza, O, or S. The shortest chain of atoms in LinkA that connects G-S—S—and —O— in formula (I) or (II) may be greater than or equal to two (e.g., greater than or equal to three; preferably, greater than or equal to four). The shortest chain of atoms in LinkA that connects G-S—S— and —O— in formula (I) or (II) may be less than or equal to 10 (e.g., less than or equal to 6; preferably less than or equal to five). In a non-limiting example, the shortest chain of atoms in LinkA that connects G-S—S— and —O— in formula (I) or (II) may be four or five. Non-limiting examples of LinkA include optionally substituted C₆₋₁₄ aryl C₁₋₆ alkylene, e.g., phenylene-ethylene, and optionally substituted C₂₋₁₀ alkylene, e.g., butylene. LinkA may include a bulky group proximal to the disulfide group.

Functional Cap Groups

A functional cap group may be a blocking group, a delivery domain, or a dye. Functional cap groups of the invention may have one or more desirable functions, e.g., protection of the disulfide group against reactivity of a phosphorus (III) atom during the synthesis of the compounds of the invention (e.g., by including a bulky blocking group). Other non-limiting examples of the desirable functions include: (1) providing a capability for delivery to a specific tissue (e.g., by including a targeting moiety); (2) providing a capability for visualizing the tissues to which the mononucleotide of the invention is delivered (e.g., by including a dye); (3) enhancing a capability for the escape from an intracellular compartment, such as endosome (e.g., by including an endosomal escape moiety); (4) enhancing the efficacy of transmembrane transport into the target cell (e.g., by including a cell penetrating peptide). A function cap group can also be used to modify solubility or bioavailability of the mononucleotide. This function can be achieved independently of the capability to deliver the mononucleotide of the invention to a specific tissue. A functional cap group can be an intermediate prior to conjugation of any of the delivery domains.

The functional cap group can fulfill one or more of these features by incorporating the moieties that provide each desired function. All types of functional cap groups (e.g., a blocking group or a delivery domain), when bonded to the phosphorus (V) atom, mask the negative charge of mononucleoside phosphate, which is released upon hydrolysis of the bond between the functional cap group and the phosphorus (V) atom in vivo.

### Sugars

Sugars included in the mononucleotides of the present invention can be monosaccharides having at least 5 carbon atoms, which may be linear, branched, or cyclic. In particular, the sugar can be a ribose or a modification thereof, e.g., a 2-deoxyribose, 2-methylribose, 2-methyl-2-deoxyribose. The 2-deoxyribose sugars can include a halogen (e.g., F) or optionally substituted C₁₋₆ alkoxy (e.g., methoxy or methoxyethoxy) at position 2.

The sugar can be a compound of formula (III):

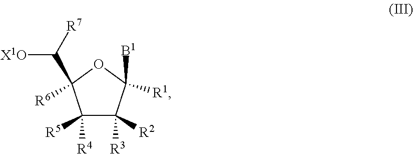

where
X¹ is a bond to the phosphorus (V) atom of a phosphate group;
B¹ is a bond to a nucleobase;
R¹ is H, azido, cyano, optionally substituted C₁₋₆ alkyl, optionally substituted C₂₋₆ alkenyl, or optionally substituted C₂₋₆ alkynyl;
each of R² and R³ is independently H, amino, azido, optionally substituted C₁₋₆ alkyl (e.g., methyl), optionally substituted C₁₋₆ heteroalkyl, optionally substituted C₂₋₆ alkenyl, optionally substituted C₂₋₆ alkynyl, halo (e.g., F), cyano, hydroxy, or optionally substituted C₁₋₆ alkoxy,
R⁴ is hydroxy, optionally substituted C₁₋₆ alkoxy (e.g., alkoxy optionally substituted with ═O and/or amino (e.g., —NH₂)), optionally substituted amino, azido, or —O—X², where X² is a bond to the phosphorus (V) atom;
R⁵ is H, optionally substituted C₁₋₆ alkyl, optionally substituted C₁₋₆ heteroalkyl, optionally substituted C₂₋₆ alkenyl, optionally substituted C₂₋₆ alkynyl, or cyano;
R⁶ is H, azido, cyano, halo (e.g., F), optionally substituted C₁₋₆ alkyl, optionally substituted C₂₋₆ alkenyl, or optionally substituted C₂₋₆ alkynyl; and
R⁷ is H or optionally substituted C₁₋₆ alkyl (e.g., Me).

In certain embodiments, if one of R² and R³ is halo, the other is not amino, hydroxy, or optionally substituted C₁₋₆ alkoxy. In other embodiments, at least one of R² and R³ is not H.

The mononucleotide of the invention can have a structure of formula (II):

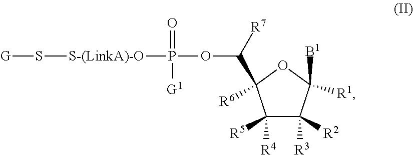

or a pharmaceutically acceptable salt or a phosphorus diastereomer thereof,

where
G is a functional cap group;
LinkA is a linker;
B¹ is a nucleobase;
R¹ is H, azido, cyano, optionally substituted C₁₋₆ alkyl, optionally substituted C₂₋₆ alkenyl, or optionally substituted C₂₋₆ alkynyl;
each of R² and R³ is independently H, amino, azido, optionally substituted C₁₋₆ alkyl (e.g., methyl), optionally substituted C₁₋₆ heteroalkyl, optionally substituted C₂₋₆ alkenyl, optionally substituted C₂₋₆ alkynyl, halo (e.g., F), cyano, hydroxy, or optionally substituted C₁₋₆ alkoxy,
G¹ is an optionally substituted amino, optionally substituted C₁₋₆ alkoxy, optionally substituted C₆₋₁₄ aryloxy, or optionally substituted C₁₋₉ heteroaryloxy, and R⁴ is hydroxy, optionally substituted C₁₋₆ alkoxy (e.g., alkoxy optionally substituted with ═O and/or amino (e.g., —NH₂)), optionally substituted amino, or azido, or G¹ and R⁴ combine to form —O—;
R⁵ is H, optionally substituted C₁₋₆ alkyl, optionally substituted C₁₋₆ heteroalkyl, optionally substituted C₂₋₆ alkenyl, optionally substituted C₂₋₆ alkynyl, or cyano;
R⁶ is H, azido, cyano, halo (e.g., F), optionally substituted C₁₋₆ alkyl, optionally substituted C₂₋₆ alkenyl, or optionally substituted C₂₋₆ alkynyl; and
R⁷ is H or optionally substituted C₁₋₆ alkyl (e.g., Me).

In certain embodiments, if one of R² and R³ is halo, the other is not amino, hydroxy, or optionally substituted C₁₋₆ alkoxy. In other embodiments, at least one of R² and R³ is not H.

### Nucleobases

Nucleobases included in the mononucleotides of the present invention can be modified or unmodified nucleobases. Unmodified nucleobases can be a purine base (e.g., adenine (A) or guanine (G)) or a pyrimidine base (e.g., thymine (T), cytosine (C), or uracil (U)). Modified nucleobases can be protected versions of the purine or pyrimidine base, in which one or more oxygen and/or nitrogen atoms is protected with an appropriate protecting group or is present as a prodrug moiety. In a non-limiting example, the nucleobase can be uracil, cytosine, adenosine, or guanosine, or a modification thereof (e.g., 2-amino-6-alkoxypurine).

Nucleobases may include one or more positions enriched in an isotope heavier than the atomic weight of an element. For example, a nucleobase may include a nitrogen atom position that is enriched in ¹⁵N. In some embodiments, the nucleobase is cytosine having an exocyclic amino group enriched in 15_{N.}

The mononucleotides of the invention have a modular structure, which allows for variation of portions of the molecule (e.g., variation of functional cap groups, such as inclusion of targeting moieties) without substantially affecting the sulfur-sulfur bond cleavage mechanism. The inclusion of the targeting moieties in the compounds of the invention may decrease the minimum effective concentration required for the pharmaceutical activity of the mononucleotide. For example, by including a targeting moiety specific to liver cells (e.g., GaINAc, mannose, or a lipid), the compounds of the invention may be specifically delivered to liver cells even if administered systemically (e.g., orally, topically, or intravenously).

Non-limiting examples of the mononucleotides of the invention include:

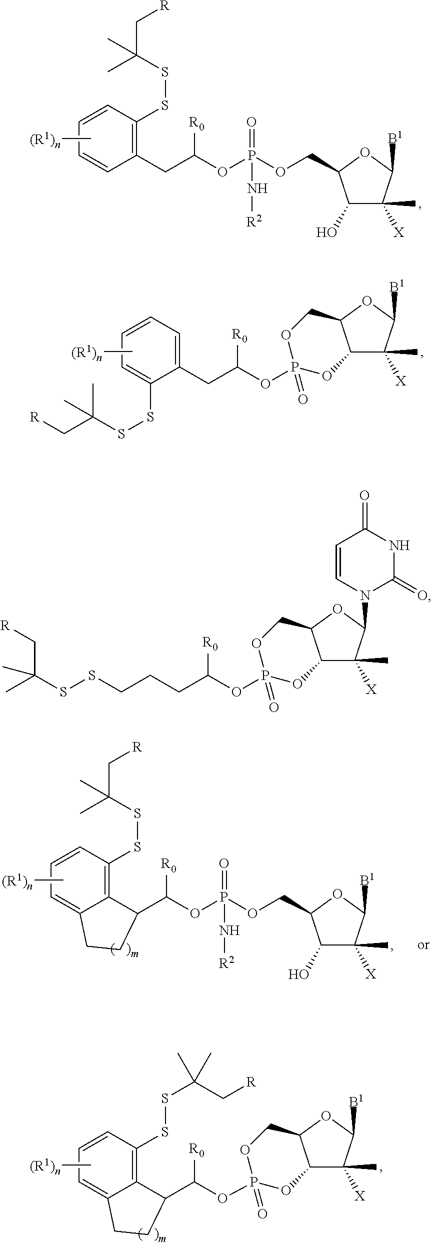

or a pharmaceutically acceptable salt or a phosphorus diastereomer thereof,
in which

X is F, OH, or optionally substituted C₁₋₆ alkoxy (e.g., OMe);

R is H, OH, or optionally substituted amino (e.g., NMe₂);

R₀ is H or optionally substituted C₁₋₆ alkyl (e.g., Me);

each R¹ is independently halogen, C₁₋₆ alkyl (e.g., Me), C₃₋₈ cycloalkyl (e.g., cyclopentyl), or C₁₋₉ heterocyclyl (e.g., C₅ including one heteroatom: N, O, or S);

n is 0, 1, 2, 3, or 4;

m is 1, 2, or 3;

R² is optionally substituted C₁₋₆ alkyl (e.g., benzyl or (R)-1-isopropoxycarbonyl-ethyl); and

B¹ is a nucleobase (e.g., uracil, cytosine, adenosine, guanosine, (2-amino-6-methoxy)purin-9-yl, or (2-amino-6-ethoxy)purin-9-yl).

In some embodiments, m is 2.

In particular, the mononucleotide of the invention can be one of the following compounds:

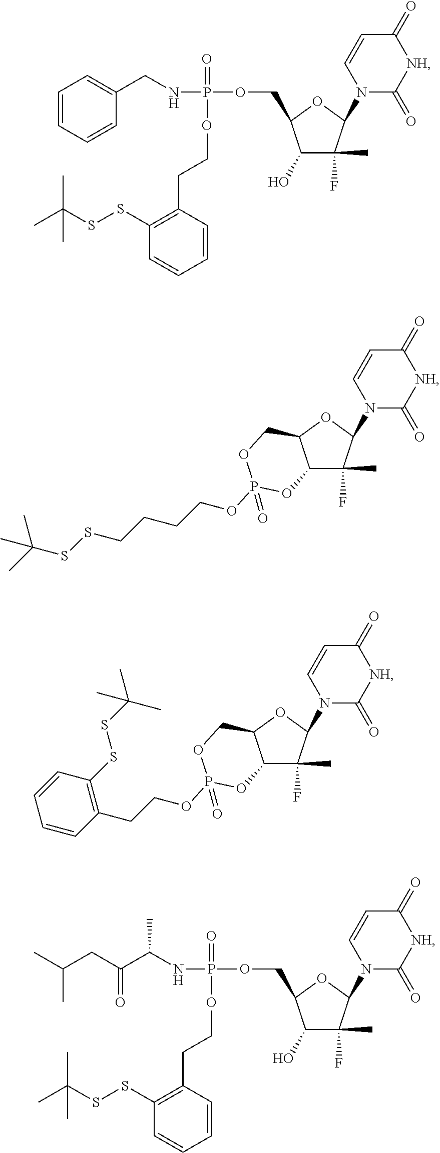

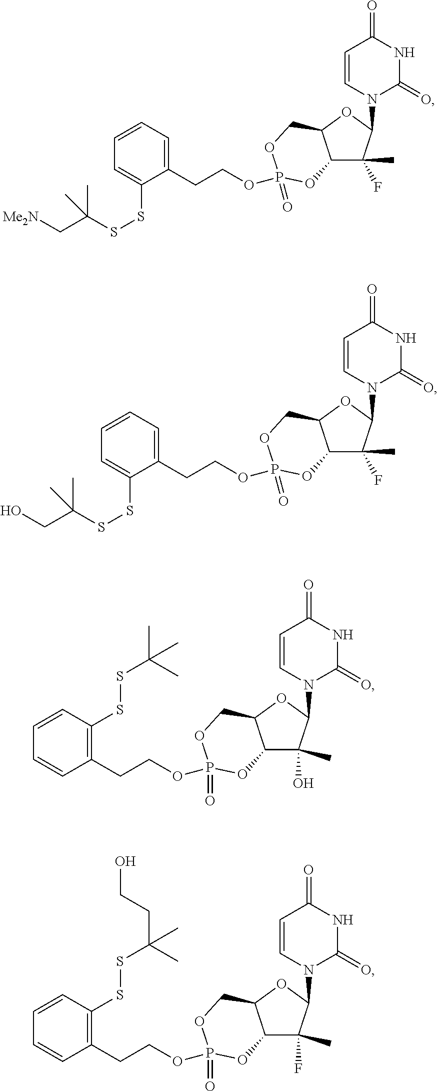

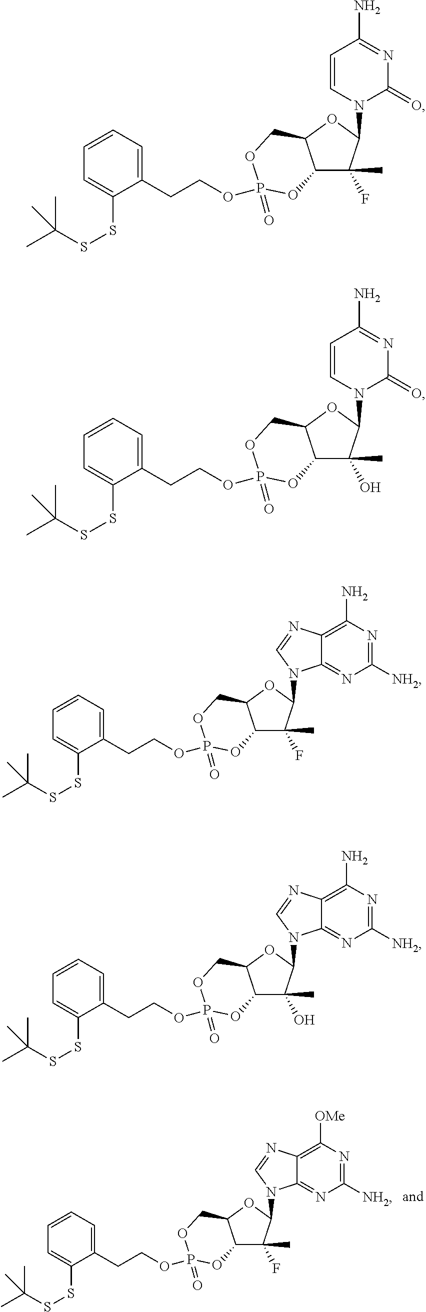

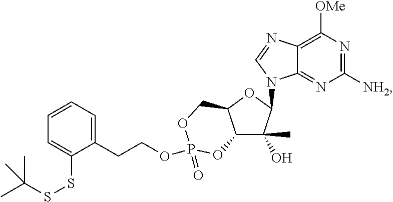

or a pharmaceutically acceptable salt or a phosphorus diastereomer thereof,

Non-limiting examples of the mononucleotides of the invention including a delivery domain are as follows:

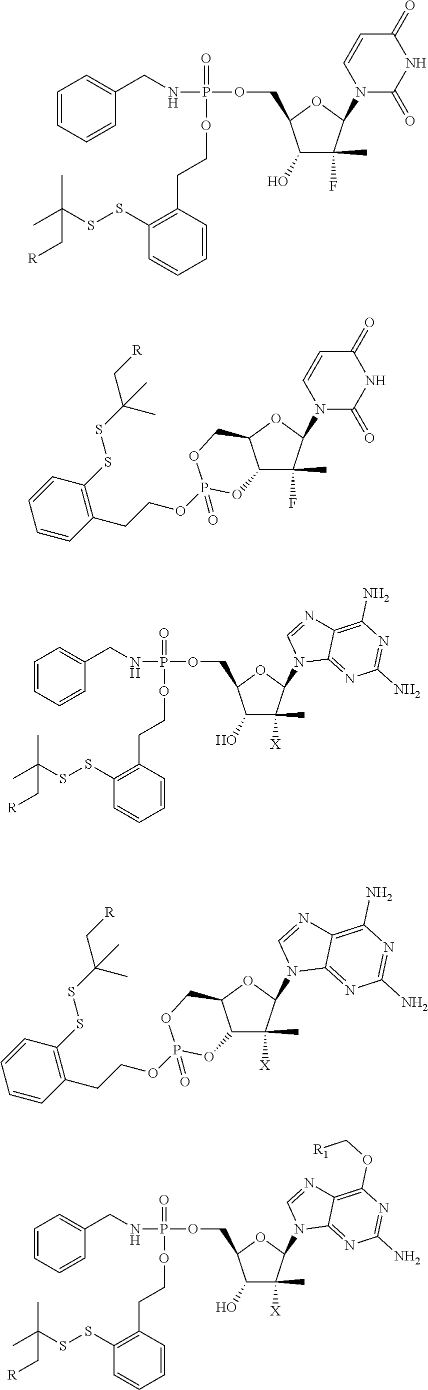

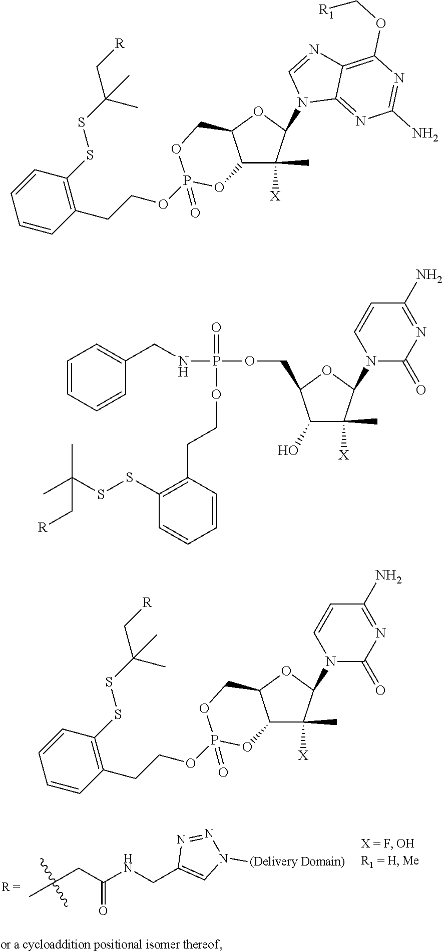

or a pharmaceutically acceptable salt or a phosphorus diastereomer thereof,

Delivery Domain can be, e.g., a targeting moiety (e.g., GaINAc, Mannose, Lipid, etc.), a cell penetrating peptide, or an endosomal escape moiety.

### Blocking Groups

The blocking groups included in the compounds of the invention may have a molecular weight greater than or equal to 43 Da (e.g., greater than or equal to 57 Da). The blocking groups may have a molecular weight of less than or equal to 10 kDa (e.g., less than or equal to 3 kDa or less than or equal to 300 Da). The structures within the blocking group may be inert to spontaneous reactivity under intracellular physiological conditions. The blocking group may contain a bulky group proximal to the disulfide (e.g., a blocking group may include a branched optionally substituted C₃₋₁₀ alkylene (e.g., this blocking group may be a branched optionally substituted C₃₋₁₀ alkyl)), particularly in those mononucleotides of the invention, which lack a bulky group proximal to the disulfide on the linker connecting the disulfide to the phosphorus (V) atom. Non-limiting examples of blocking groups include optionally substituted C₃₋₁₀ alkyl (e.g., t-Bu; 2-hydroxy-1,1-dimethyl-ethyl; and 2-dimethylamino-1,1-dimethyl-ethyl).

### Delivery Domains

The inclusion of a delivery domain in the mononucleotide of the invention may facilitate one or more of targeting a specific tissue type, a cellular uptake of the mononucleotide of the invention, an intracellular release of the mononucleoside or mononucleoside phosphate inside a cell (e.g., from an intracellular compartment, such as an endosome) after the cellular uptake, and detection of the delivery of the mononucleoside or mononucleoside phosphate into the targeted cell. Thus, a delivery domain may be a targeting moiety, a dye, an endosomal escape moiety, or a cell penetrating peptide.

A targeting moiety (e.g., extracellular targeting moiety) is any moiety that specifically binds or reactively associates or complexes with a receptor or other receptive moiety associated with a given target cell population (e.g., liver cells or lymphocytes). Non-limiting examples of targeting moieties for liver cells include carbohydrates (e.g., GaINAc or mannose) and lipids. Non-limiting examples of targeting moieties for lymphocytes include anti-CD3 antibodies (e.g., otelixizumab, teplizumab, and visilizumab), anti-CD4 antibodies (e.g., OKT4 or RPA-T4, available from eBioscience, San Diego, Calif.), anti-CD8 antibodies (e.g., OKT8 or SK1, available from eBioscience, San Diego, Calif.), anti-CD16 antibodies (e.g., CB16 or B73.1, available from eBioscience, San Diego, Calif.), and anti-CD19 antibodies (e.g., HIB19 available from eBioscience, San Diego, Calif.). Targeting moieties for other cells are known in the art. Some of the extracellular targeting moieties of the invention are described herein. In one embodiment, the targeting moiety is a receptor binding domain. In another embodiment, the targeting moiety is or specifically binds to a protein selected from the group comprising insulin, insulin-like growth factor receptor 1 (IGF1R), IGF2R, insulin-like growth factor (IGF; e.g., IGF 1 or 2), mesenchymal epithelial transition factor receptor (c-met; also known as hepatocyte growth factor receptor (HGFR)), hepatocyte growth factor (HGF), epidermal growth factor receptor (EGFR), epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-α), TNF-β, folate receptor (FOLR), folate, transferrin, transferrin receptor (TfR), mesothelin, Fc receptor, c-kit receptor, c-kit, an integrin (e.g., an α4 integrin or a β-1 integrin), P-selectin, sphingosine-1-phosphate receptor-1 (S1PR), hyaluronate receptor, leukocyte function antigen-1 (LFA-1), CD4, CD11, CD18, CD20, CD25, CD27, CD52, CD70, CD80, CD85, CD95 (Fas receptor), CD106 (vascular cell adhesion molecule 1 (VCAM1), CD166 (activated leukocyte cell adhesion molecule (ALCAM)), CD178 (Fas ligand), CD253 (TNF-related apoptosis-inducing ligand (TRAIL)), ICOS ligand, CCR2, CXCR3, CCR5, CXCL12 (stromal cell-derived factor 1 (SDF-1)), interleukin 1 (IL-1), IL-1ra, IL-2, IL-3, IL-4, IL-6, IL-7, IL-8, CTLA-4, MART-1, gp100, MAGE-1, ephrin (Eph) receptor, mucosal addressin cell adhesion molecule 1 (MAdCAM-1), carcinoembryonic antigen (CEA), Lewis^{Y}, MUC-1, epithelial cell adhesion molecule (EpCAM), cancer antigen 125 (CA125), prostate specific membrane antigen (PSMA), TAG-72 antigen, and fragments thereof. In further embodiments, the targeting moiety is erythroblastic leukemia viral oncogene homolog (ErbB) receptor (e.g., ErbB1 receptor; ErbB2 receptor; ErbB3 receptor; and ErbB4 receptor). In other embodiments, a targeting moiety may selectively bind to asialoglycoprotein receptor, a manno receptor, or a folate receptor. In particular embodiments, the targeting moiety contains one or more N-acetyl galactosamines (GaINAc), mannoses, or a folate ligand. In certain embodiments, the folate ligand has the structure:

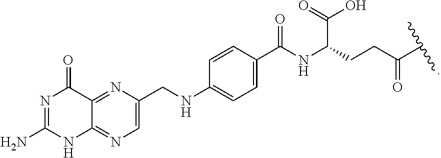 [SMILES: *C(=O)CC[C@H](NC(=O)c1ccc(NCc2cnc3[nH]c(N)nc(=O)c3n2)cc1)C(=O)O]

The targeting moiety can also be selected from bombesin, gastrin, gastrin-releasing peptide, tumor growth factors (TGF), such as TGF-α and TGF-β, and vaccinia virus growth factor (VVGF). Non-peptidyl ligands can also be used as the targeting moiety and may include, for example, steroids, carbohydrates, vitamins, and lectins. The targeting moiety may also be selected from a polypeptide, such as somatostatin (e.g., a somatostatin having the core sequence cyclo[Cys-Phe-D-Trp-Lys-Thr-Cys] (SEQ ID NO: 6), and in which, for example, the C-terminus of the somatostatin analog is: Thr-NH₂), a somatostatin analog (e.g., octreotide and lanreotide), bombesin, a bombesin analog, or an antibody, such as a monoclonal antibody.

Endosomal escape moieties enhance the release of endosomal contents or allow for the escape of a molecule from an internal cellular compartment such as an endosome. Exemplary endosomal escape moieties include chemotherapeutics (e.g., quinolones such as chloroquine); fusogenic lipids (e.g., dioleoylphosphatidyl-ethanolamine (DOPE)); and polymers such as polyethylenimine (PEI); poly(beta-amino ester)s; peptides or polypeptides such as polyarginines (e.g., octaarginine) and polylysines (e.g., octalysine); proton sponges, viral capsids, and peptide transduction domains as described herein. For example, fusogenic peptides can be derived from the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion protein of the Sendai virus; the transmembrane glycoprotein of the Semliki forest virus; the fusion protein of the human respiratory syncytial virus (RSV); the fusion protein of the measles virus; the fusion protein of the Newcastle disease virus; the fusion protein of the visna virus; the fusion protein of murine leukemia virus; the fusion protein of the HTL virus; and the fusion protein of the simian immunodeficiency virus (SIV). Other moieties that can be employed to facilitate endosomal escape are described in Dominska et al., Journal of Cell Science, 123(8):1183-1189, 2010. Non-limiting examples of endosomal escape moieties are provided in Table 1.

A cell penetrating peptide is a short polypeptide (e.g., a polypeptide of 4 to 50 amino acids) that facilitates cellular uptake of the mononucleotide of the invention. A cell penetrating peptide may contain a cationic Peptide Transduction Domain (PTD), such as TAT or (Arg₈) (Snyder and Dowdy, 2005, Expert Opin. Drug Deliv. 2, 43-51). PTDs can be used to deliver a wide variety of cargo (Schwarze et al., 1999, Science 285, 1569-1572; Eguchi et al., 2001, J. Biol. Chem. 276, 26204-26210; and Koppelhus et al., 2002, Antisense Nucleic Acid Drug Dev. 12, 51-63), including the mononucleotides described herein. Cationic PTDs enter cells by macropinocytosis, a specialized form of fluid phase uptake that all cells perform. Non-limiting examples of cell-penetrating peptides are provided in Table 1.

TABLE 1

| Com- | SEQ | | C- | | MW |
|---|---|---|---|---|---|
| pound | ID | | Term- | MW | Ob- |
| # | NO: | Structure | inus | Calcd | serv |
| P22 | 1 | N₃ GGRKKRRQRRR-Peg24- | CONH₂ | 6459 | 6450 |
| | | GGRKKRRQRRR-Peg24- | | | |
| | | GGRKKRRQRRR | | | |
| P27 | 2 | GGLHKLLHHLLHHLHKLLHHLHHLLHKL | CONH₂ | 3382 | 3380 |
| P28 | 3 | GGACTGSTQHQCG | CONH₂ | 1205 | 1203 |
| P29 | 4 | GGLIRLWSHLIHIWFQNRRLKWKKK | CONH₂ | 3214 | 3211 |
| P31 | 5 | GGIGAVLKVLTTGLPALISWIKRKRQQ | CONH₂ | 2904 | 2903 |

In Table 1: compound P22 includes a cell-penetrating peptide; and compounds P27, P28, P29, and P31 include endosomal escape moieties.

### Dyes

Dyes may be included in the functional cap groups for the purpose of visualization of uptake. or monitoring the movement of the mononucleotide of the invention inside a cell (e.g., using Fluorescence Recovery After Photobleaching (FRAP)). Dyes known in the art may be included in a functional cap group. Non-limiting examples of useful structures that can be included in dyes include FITC, RD1, allophycocyanin (APC), aCFTM dye (Biotium, Hayward, Calif.), BODIPY (Invitrogen of Life Technologies, Carlsbad, Calif.), AlexaFluor® (Invitrogen of Life Technologies, Carlsbad, Calif.), DyLight Fluor (Thermo Scientific Pierce Protein Biology Products, Rockford, Ill.), ATTO (ATTO-TEC GmbH, Siegen, Germany), FluoProbe (Interchim SA, Motluçon, France), and Abberior Probes (Abberior GmbH, Göttingen, Germany).

### Unmasking of Mononucleotides

Without being bound by theory, the mononucleotides of the invention can be unmasked by intracellular reduction of the disulfide, following by intramolecular cyclization. Additional moieties on the phosphorous atom, e.g., alkoxy or amino, can be released by known mechanisms, e.g., enzymatically (e.g., through the action of phosphoramidase, phosphodiesterase, or general hydrolysis). One non-limiting example of the disulfide bond cleavage inside a cell with subsequent release of, e.g., an unmasked mononucleotide, is shown in Scheme 1.

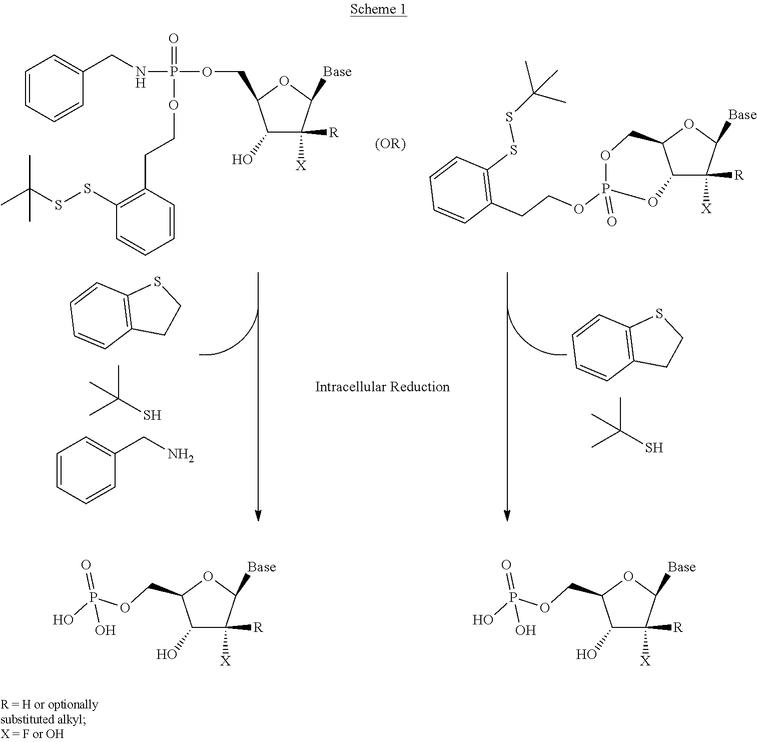

### Synthesis of the Mononucleotides of the Invention

A mononucleotide of the invention can be prepared according to the methods described herein or according to the methods known in the art. A non-limiting example of the synthesis of a mononucleotide of the invention is shown in Scheme 2.

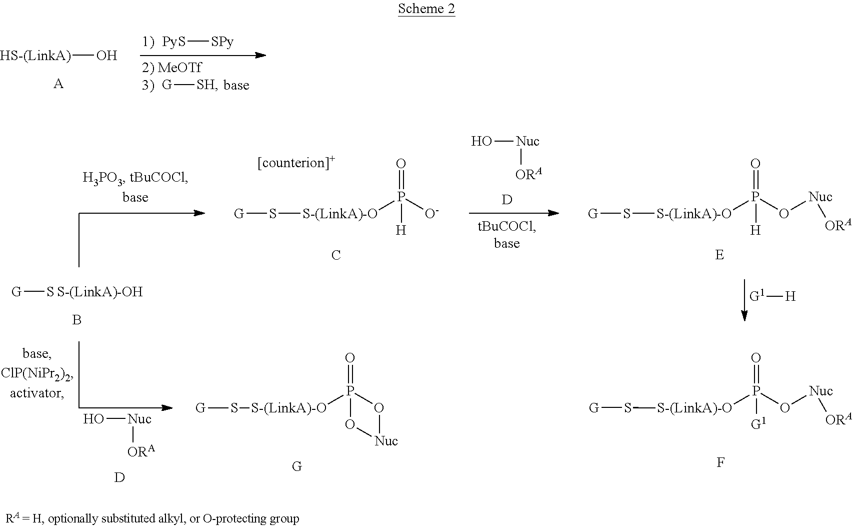

In Scheme 2, HO-Nuc-OR^{A} is a mononucleoside, which may be unprotected (e.g., R^{A} is H or optionally substituted alkyl) or protected with an O-protecting group. One of skill in the art will recognize that the synthesis of compound G requires R^{A} to be H. One of skill in the art will also recognize that the synthesis of compound F permits R^{A} to be any group within the scope of the present invention, including an O-protecting group, which, if desired, may be removed at the end of the synthesis.

As shown in Scheme 2, compound A can be subjected to a metathesis reaction with 2,2′-dipyridyldisulfide (PyS-SPy) to afford a mixed disulfide intermediate, which, upon treatment with an electrophile (e.g., MeOTf) followed by G-SH in the presence of a base (e.g., a trialkylamine base, such as Hünig's base (DIEA)), can furnish compound B.

Compound B can be used to prepare compounds of the invention that include a phosphorus (V) atom having only one (e.g., compound F) or two (e.g., compound G) valencies bonded to a mononucleoside. Thus, preparation of compound F can be achieved according to the following sequence of reactions. Compound B can be reacted with phosphorous acid in the presence of a base (e.g., organic base, such as pyridine) and pivaloyl chloride to furnish compound C, which upon reaction with compound D in the presence of pivaloyl chloride and a base (e.g., pyridine) can yield compound E. The counterion in compound C may originate in the base employed in the reaction or may be provided upon quench. Treatment of compound E with G¹-H (H is attached to a heteroatom, such as N or O) can provide compound F. Compound G can be prepared by reacting compound B with compound D (R^{A}═H) in the presence of a base (e.g., trialkylamine base, such as Hünig's base (DIEA)), an activator (e.g., 4,5-dicyanoimidazole), and CIP(NiPr₂)₂.

In the reactions described above, it may be necessary to protect reactive functional groups (e.g., hydroxy, amino, thio, or carboxy groups) to avoid their unwanted participation in the reactions. The incorporation of such groups, and the methods required to introduce and remove them are known to those skilled in the art (for example, Greene, supra). The deprotection step may be the final step in the synthesis such that the removal of protecting groups affords compounds of the invention. Starting materials used in any of the schemes above can be purchased or prepared by methods described in the chemical literature, or by adaptations thereof, using methods known by those skilled in the art. The order in which the steps are performed can vary depending on the groups introduced and the reagents used, but would be apparent to those skilled in the art.

### Pharmaceutical Compositions

The compounds used in the methods described herein are preferably formulated into pharmaceutical compositions for administration to human subjects in a biologically compatible form suitable for administration in vivo. Pharmaceutical compositions typically include a compound as described herein and a pharmaceutically acceptable excipient.

For human use, a mononucleotide of the invention can be administered alone or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of compounds of Formula (I) or (II) into preparations which can be used pharmaceutically.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6^{th} Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21^{st} Ed., Gennaro, Ed., Lippencott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

### Dosages

The dosage of the compound used in the methods described herein, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds used in the methods described herein may be administered initially in a suitable dosage that may be adjusted as required, depending on the clinical response. In general, a suitable daily dose of a mononucleotide of the invention will be that amount of the compound that is the lowest dose effective to produce a therapeutic effect. Such an effective dose will generally depend upon the factors described above.

A mononucleotide of the invention may be administered to the patient in a single dose or in multiple doses. When multiple doses are administered, the doses may be separated from one another by, for example, 1-24 hours, 1-7 days, 1-4 weeks, or 1-12 months. The compound may be administered according to a schedule or the compound may be administered without a predetermined schedule. An active compound may be administered, for example, 1, 2, 3, 4, 5, 6, 7, 8, 9, 10, 11, or 12 times per day, every 2^{nd}, 3^{rd}, 4^{th}, 5^{th}, or 6^{th} day, 1, 2, 3, 4, 5, 6, or 7 times per week, 1, 2, 3, 4, 5, or 6 times per month, or 1, 2, 3, 4, 5, 6, 7, 8, 9, 10, 11, or 12 times per year. It is to be understood that, for any particular subject, specific dosage regimes should be adjusted over time according to the individual need and the professional judgment of the person administering or supervising the administration of the compositions.

While the attending physician ultimately will decide the appropriate amount and dosage regimen, an effective amount of a mononucleotide of the invention may be, for example, a total daily dosage of, e.g., between 0.05 mg and 3000 mg of any of the compounds described herein. Alternatively, the dosage amount can be calculated using the body weight of the patient. Such dose ranges may include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

In the methods of the invention, the time period during which multiple doses of a mononucleotide of the invention are administered to a patient can vary. For example, in some embodiments doses of the compounds of the invention are administered to a patient over a time period that is 1-7 days; 1-12 weeks; or 1-3 months. In other embodiments, the compounds are administered to the patient over a time period that is, for example, 4-11 months or 1-30 years. In other embodiments, the compounds are administered to a patient at the onset of symptoms. In any of these embodiments, the amount of compound that is administered may vary during the time period of administration. When a compound is administered daily, administration may occur, for example, 1, 2, 3, 4, 5, 6, 7, 8, 9, 10, 11, or 12 times per day.

### Formulations

A compound identified as capable of treating any of the conditions described herein, using any of the methods described herein, may be administered to patients or animals with a pharmaceutically-acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer the identified compound to patients suffering from a disease in which necrosis occurs. Administration may begin before the patient is symptomatic.

The compounds or pharmaceutical compositions thereof, may be administered to a patient in a variety of forms depending on the selected route of administration, as will be understood by those skilled in the art. The compounds used in the methods described herein may be administered, for example, by enteral or parenteral administration. Enteral administration may be oral route of administration. Parenteral administration may include intramuscular, intravenous, intraarterial, intracranial, subcutaneous, intraorbital, intraventricular, intraspinal, intrathecal, intraperitoneal, rectal, and topical routes of administration. Topical route of administration may include transdermal, intradermal, intranasal, intrapulmonary, buccal, and sublingual routes of administration. The pharmaceutical compositions are formulated according to the selected route of administration. Parenteral administration may be by continuous infusion over a selected period of time. The compounds desirably are administered with a pharmaceutically acceptable carrier. Pharmaceutical formulations of the compounds described herein formulated for treatment of the disorders described herein are also part of the present invention.

### Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, maltodextrin, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Formulations for Buccal Administration

Dosages for buccal or sublingual administration typically are 0.1 to 500 mg per single dose as required. In practice, the physician determines the actual dosing regimen which is most suitable for an individual patient, and the dosage varies with the age, weight, and response of the particular patient. The above dosages are exemplary of the average case, but, in certain individual instances, higher or lower dosages are merited, and such are within the scope of this invention.

For buccal administration, the compositions may take the form of tablets, lozenges, etc. formulated in a conventional manner. Liquid drug formulations suitable for use with nebulizers and liquid spray devices and electrohydrodynamic (EHD) aerosol devices will typically include a mononucleotide of the invention with a pharmaceutically acceptable carrier. Preferably, the pharmaceutically acceptable carrier is a liquid, e.g., alcohol, water, polyethylene glycol, or a perfluorocarbon. Optionally, another material may be added to alter the aerosol properties of the solution or suspension of compounds of the invention. Desirably, this material is liquid, e.g., an alcohol, glycol, polyglycol, or a fatty acid. Other methods of formulating liquid drug solutions or suspension suitable for use in aerosol devices are known to those of skill in the art (see, e.g., Biesalski, U.S. Pat. No. 5,112,598 and Biesalski, U.S. Pat. No. 5,556,611, each of which is herein incorporated by reference).

### Formulations for Nasal or Inhalation Administration

The compounds may also be formulated for nasal administration. Compositions for nasal administration also may conveniently be formulated as aerosols, drops, gels, and powders. The formulations may be provided in a single or multidose form. In the case of a dropper or pipette, dosing may be achieved by the patient administering an appropriate, predetermined volume of the solution or suspension. In the case of a spray, this may be achieved, for example, by means of a metering atomizing spray pump.

The compounds may further be formulated for aerosol administration, particularly to the respiratory tract by inhalation and including intranasal administration. The compound will generally have a small particle size for example on the order of five (5) microns or less. Such a particle size may be obtained by means known in the art, for example by micronization. The active ingredient is provided in a pressurized pack with a suitable propellant, e.g., a chlorofluorocarbon (CFC), for example, dichlorodifluoromethane, trichlorofluoromethane, or dichlorotetrafluoroethane, or carbon dioxide, or other suitable gas. The aerosol may conveniently also contain a surfactant, e.g., lecithin. The dose of drug may be controlled by a metered valve. Alternatively, the active ingredients may be provided in a form of a dry powder, e.g., a powder mix of the compound in a suitable powder base, e.g., lactose, starch, starch derivatives (e.g., hydroxypropylmethyl cellulose), or polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, e.g., a single dose nasal inhaler or an aerosol dispenser fitted with a metering valve which is intended for disposal after use. Where the dosage form comprises an aerosol dispenser, it will contain a propellant, which can be a compressed gas, e.g., compressed air or an organic propellant, e.g., fluorochlorohydrocarbon. The aerosol dosage forms can also take the form of a pump-atomizer.

### Formulations for Parenteral Administration

The compounds described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:
(1) “Drug Injection”: a liquid preparation that is a drug substance (e.g., a compound of Formula (I) or (II)), or a solution thereof;
(2) “Drug for Injection”: the drug substance (e.g., a compound of Formula (I) or (II)) as a dry solid that will be combined with the appropriate sterile vehicle for parenteral administration as a drug injection;
(3) “Drug Injectable Emulsion”: a liquid preparation of the drug substance (e.g., a compound of Formula (I) or (II)) that is dissolved or dispersed in a suitable emulsion medium;
(4) “Drug Injectable Suspension”: a liquid preparation of the drug substance (e.g., a compound of Formula (I) or (II)) suspended in a suitable liquid medium; and
(5) “Drug for Injectable Suspension”: the drug substance (e.g., a compound of Formula (I) or (II)) as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21^{st} Ed., Gennaro, Ed., Lippencott Williams & Wilkins (2005)and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

### Methods of Treating

The mononucleotides of the invention can be used for the treatment of a disease or condition treatable by a mononucleotide or a mononucleoside therapy (e.g., RNA viral infections (e.g., HIV or hepatitis C)), as the mononucleotides of the invention can include a mononucleoside or mononucleotide that, upon unmasking in vivo, is known to treat the disease or condition (e.g., the RNA viral infection (e.g., HIV or hepatits C)). The methods of the invention include a method of treating a disease or condition treatable by a mononucleotide or a mononucleoside therapy (e.g., an RNA viral infection (e.g., HIV or hepatitis C)) by administering the mononucleotide of the invention or a pharmaceutical composition of the invention to a subject (e.g., a human) in need thereof. The formulations, routes of administration, and dosages can be as described above. The methods of the invention also include a method of delivering a mononucleoside or a mononucleotide to a cell (e.g., a liver cell or a lymphocyte) by contacting the cell with the mononucleotide of the invention.

The following examples are meant to illustrate the invention. They are not meant to limit the invention in any way.

### EXAMPLES

### Example 1

### Preparation of the Compounds of the Invention

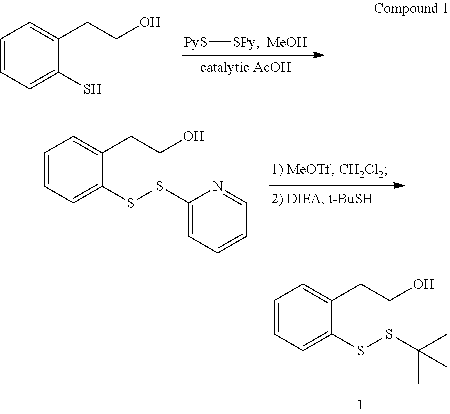

To a solution of dithiodipyridine (52.0 g, 236.3 mmol) and acetic acid (3.0 mL) in methanol (200 mL) at room temperature was added a solution of 2-(2-hydroxyethyl)thiophenol (14.6 g, 94.5 mmol) in methanol (50 mL) and stirred overnight. Volatiles were removed, and to the residue, were added 100 mL diethyl ether, and the separated solids were filtered and washed with diethyl ether (3×50 mL). The combined ether washings evaporated to give crude product which on flash silica gel column purification using ISCO companion (ethyl acetate/hexane, 0-50%) gave 14.1 g (57%) of 2-(2-hydroxyethyl)phenyl pyridyl disulfide. ¹H NMR (500 MHz, CDCl₃): δ8.48 (1H, d, J5.0 Hz), 7.65-7.60 (3H, m), 7.25-7.18 (3H, m), 7.13-7.10 (1H, m), 3.96 (2H, t, J6.5 Hz), 3.17 (1H, t, J6.5 Hz)

To a solution of 2-(2-hydroxyethyl)phenyl pyridyl disulfide (4.5 g, 17.0 mmol) in 30.0 mL of dichloromethane was added MeOTf drop wise at room temperature. The reaction mixture was stirred for 10 minutes before tert-butyl mercaptan (1.9 mL, 17.0 mmol) and N,N-diisopropylethylamine (6.0 mL, 34.0 mmol) were added. The reaction mixture was stirred for another 30 min at room temperature before being condensed in vacuo. The crude mixture was purified by silica gel column chromatography using ethyl acetate/hexane solvent system (0-30% gradient on Combi Flash Rf Instrument) to give compound 1 as colorless oil (2.5 g, 61% yield). ¹H NMR (500 MHz): δ7.84 (1H, d, J5.0 Hz), 7.25-7.13 (3H, m), 3.92 (2H, t, J7.0 Hz), 3.12 (2H, t, J7.0 Hz), 1.30 (9H, s)

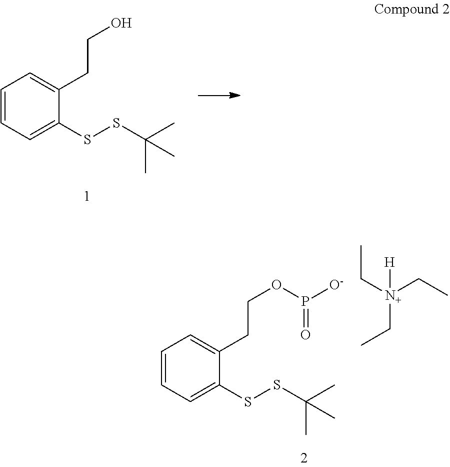

Phosphorous acid (1.69 g, 20.6 mmol) was co-evaporated three times with anhydrous pyridine and then re-dissolved in 10 mL of anhydrous pyridine. To the mixture was added alcohol 1 (0.5 g, 2.06 mmol), and the resulting mixture was stirred for 10 min and then cooled to 0° C. Pivaloyl chloride (1.37 g, 11.33 mmol) was added to the reaction mixture, warmed to room temperature, and stirred for another 3 hrs. The reaction was quenched with triethylammonium bicarbonate buffer (5.0 mL, 1 M) and diluted with ethyl acetate (30.0 mL). After extraction with ethyl acetate (3×20.0 mL), the combined organic layers were washed with triethylammonium bicarbonate buffer (5.0 mL, 0.5M) and dried over anhydrous sodium sulfate. The volatiles was removed in vacuo to afford a residue, which was subjected to flash silica gel column purification using ISCO companion (0-10% methanol/dichloromethane containing 1% triethylamine) to give 0.49 g (58%) of compound 2 as a white solid. ¹H NMR (500 Hz, CDCl₃): δ 12.15 (1H, s), 7.80 (1H, d, J8.5 Hz), 7.20 (2H, t, J6.5 Hz), 7.11 (1H, t, J6.5 Hz), 6.81 (1H, d, J6.5 Hz), 4.18 (2H, m), 3.21 (2H, t, J7.0 Hz), 3.07 (6H, m), 1.35 (9H, t, J7.0 Hz), 1.29 (9H, s). ³¹ P NMR (202 MHz, CDCl₃): δ10.3 (s)

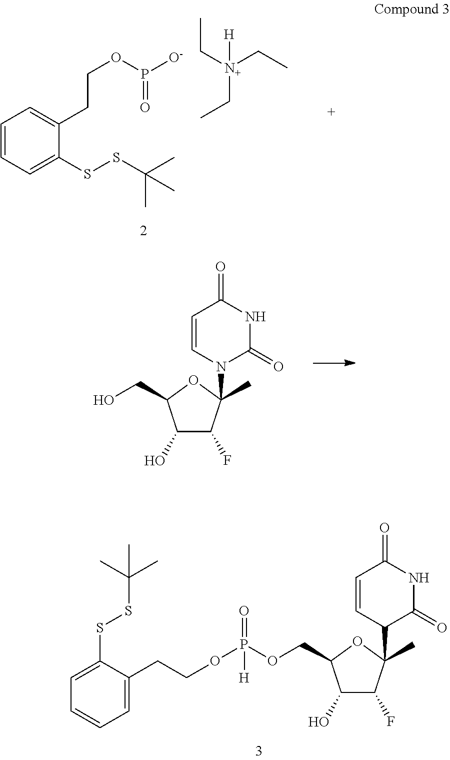

A solution of compound 2 (0.49 g, 1.20 mmol) and 2′-Me-2′-F-deoxyuridine (0.26 g, 1.0 mmol) was co-evaporated with anhydrous pyridine twice and the residue was re-dissolved in 15.0 mL of anhydrous pyridine and cooled to −15° C. To this mixture was added pivaloyl chloride (0.25 mL, 2 mmol) dropwise and stirring continued at −15° C. for 1.5 hrs. The reaction mixture was diluted with dichloromethane (30.0 mL) and quenched with aqueous ammonium chloride solution (0.5M, 20.0 mL). Organic layer separated, and the aqueous layer was extracted with dichloromethane (2×20.0 mL). The combined organic layers were washed with aqueous ammonium chloride solution (0.5M) and brine, dried over anhydrous sodium sulfate. Volatiles were removed in vacuo to afford a residue, which was subjected to flash silica gel column purification on an ISCO companion (2-10% methanol/dichloromethane containing 1% acetic acid) to give 0.24 g (44%) of compound 3 as a white solid. ¹ H NMR (500 Hz, CDCl₃): δ8.25 (1H, s), 7.84 (1H, d, J8.5 Hz), 7.56 (1H, d, J8.5 Hz), 7.39 (1H, dd, J6.5, 3.5 Hz), 7.27 (1H, m), 7.17 (2H, m), 6.85 (1H, d, J710 Hz), 5.70 (1H, dd, J8.0 Hz), 4.42-4.25 (4H, m), 4.04 (1H, d, J9.0 Hz), 5 3.90 (1H, m), 3.27 (2H, t, J6.5 Hz), 1.40 (3H, d, J22.0 Hz), 1.30 (9H, s). ³¹ P NMR (202 MHz, CDCl₃): δ14.25 (s), 14.21 (s)

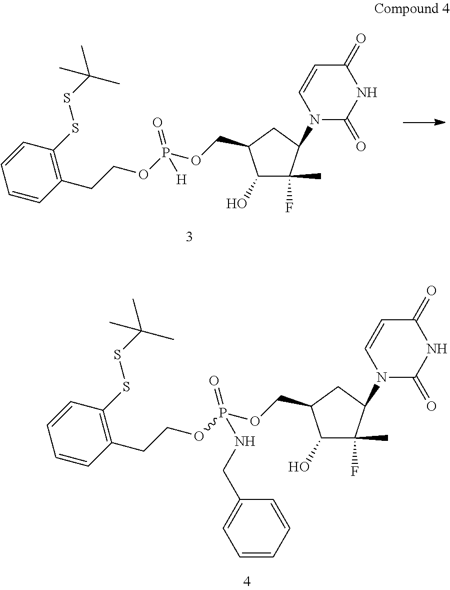 [SMILES: CC(C)(C)SSc1ccccc1CCO[PH](=O)OC[C@H]1C[C@@H](n2ccc(=O)[nH]c2=O)[C@](C)(F)[C@@H]1O ; CC(C)(C)SSc1ccccc1CCOP(=O)(NCc1ccccc1)OC[C@H]1C[C@@H](n2ccc(=O)[nH]c2=O)[C@](C)(F)[C@@H]1O]

To a solution of 3 (0.14 g, 0.26 mmol) in a mixture of dichloromethane and carbon tetrachloride (v/v=1:1, 4 mL) was added benzylamine (0.14 mL, 1.28 mmol) dropwise and the resulting mixture was stirred for 3 hrs. Volatiles were removed in vacuo to afford a residue, which was subjected to flash silica gel column purification on an ISCO companion (1-8% methanol/dichloromethane) to give 0.069 g (41%) of compound 4 (mixture of diastereomers) as a white solid. ESI MS for C₂₉H₃₇FN₃O₇PS₂ calculated 653.7, observed 654.7 [M+H]⁺. ³¹P NMR (202 MHz, CDCl₃): δ15.3 (s), 15.1 (s)

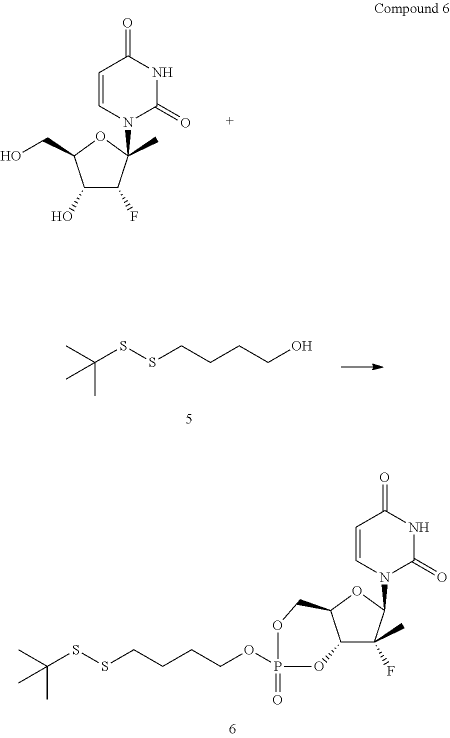

To a solution of 2′-Me-2′-F-deoxyuridine (0.13 g, 0.5 mmol) in dry dichloromethane (3.0 mL) at −78° C., bis-(N,N-diisopropylamino)-chlorophosphine (0.13 g, 0.5 mmol) in dry dichloromethane (2.0 mL) was added dropwise followed by N,N-diisopropylethylamine (0.094 mL, 0.55 mmol). The reaction mixture warmed to room temperature and was stirred for additional 1 hour. To this mixture, a solution of 4,5-dicyanoimidazole (0.054 g, 0.5 mmol) in dry acetonitrile (3.0 mL) was added, and the resulting mixture stirred for 1 hour followed by addition of acetonitrile (5.0 mL) solution of the alcohol 5 (0.097 g, 0.5 mmol), 4,5-dicyanoimidazole (0.054 g, 0.5 mmol) and stirring continued overnight. To this solution, t-butyl hydroperoxide (0.1 mL, 5-6 M in decane) was added and the mixture was stirred for additional 30 min. Volatiles were removed in vacuo to afford a residue, which was subjected to HPLC purification (acetonitrile/H₂O; 15%-65%, 30 min) to give two isomers (0.028 g of the more polar diastereomer 6A and 0.041 g of the less polar diastereomer 6B) as white solids.

Compound 6A: ¹H NMR (500 MHz, CDCl₃): δ8.50 (1H, s), 7.18 (1H, d, J8.0 Hz), 6.38 (1H, d, J19.0 Hz), 5.82 (1H, d, J7.5 Hz), 4.68 (1H, m), 4.55 (1H, m), 4.36 (1H, m), 4.25-4.15 (3H, m), 2.73 (2H, t, J6.5 Hz), 1.87-1.77 (4H, m), 1.47 (3H, d, J20.0 Hz), 1.33 (9H, s). ESI MS for C₁₈H₂₈FN₂O₇PS₂ calculated 498.5, observed 497.4 [M-H]⁺. ³¹P NMR (202 MHz, CDCl₃) δ1.7 (s)

Compound 6B: ¹H NMR (500 MHz, CDCl₃): δ7.65-7.60 (2H, m), 6.35 (1H, d, J21.0 Hz), 5.76 (1H, s, br), 4.671 (1H, m), 4.60 (1H, m), 4.32 (1H, m), 4.25-4.18 (3H, m), 2.78 (2H, t, J6.5 Hz), 1.92-1.78 (4H, m), 1.46 (3H, d, J22.0 Hz), 1.33 (9H, s). ESI MS for C₁₈H₂₈FN₂O₇PS₂ calculated 498.5, observed 497.4 [M-H]⁺. ³¹P NMR (202 MHz, CDCl₃) δ0.6(s)

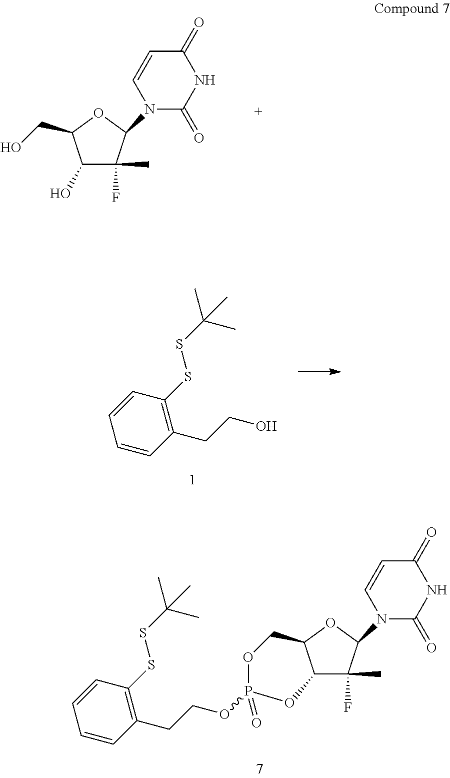

A solution of bis-(N,N-diisopropylamino)-chlorophosphine (0.13 g, 0.5 mmol) in dry dichloromethane (2.0 mL) was added dropwise to a solution of 2-Me-2′-F-uridine (0.13 g, 0.5 mmol) and N,N-diisopropylethylamine (0.094 mL, 0.55 mmol) in dry dichloromethane (3.0 mL) at −78° C. The reaction mixture warmed to room temperature and stirred for 1 hour. A solution of 4,5-dicyanoimidazole (0.054 g, 0.5 mmol) in dry acetonitrile (3.0 mL) was added, and the resulting mixture was stirred for 1 hour. To this, a solution of the alcohol 1 (0.12 g, 0.5 mmol) and 4,5-dicyanoimidazole (0.054 g, 0.5 mmol) in acetonitrile (5.0 mL) was added, and the resulting mixture was stirred overnight. t-Butyl hydroperoxide solution (0.1 mL, 5-6 M in decane) was added and the mixture was stirred for additional 30 min. Volatiles were removed in vacuo to afford a residue which was subjected to HPLC purification (acetonitrile/H₂O; 20%-75%, 30 min) to give 0.011 g of the more polar diastereomer 7A and 0.039 g of the less polar diastereomer 7B as white solids.

Compound 7A: ¹H NMR (500 MHz, CDCl₃): δ8.68 (1H, s), 7.83 (1H, d, J8.0 Hz), 7.30-7.15 (3H, m), 7.04 (1H, s), 6.29 (1H, d, J20.0 Hz), 6.02 (1H, s), 4.55-4.43 (3H, m), 4.21 (1H, td, J10.0, 4.5 Hz), 3.95-3.50 (2H, m), 3.40-3.25 (2H, m), 1.29 (9H, s), 1.29 (3H, m). ESI MS for C₂₂H₂₈FN₂O₇PS₂ calculated 546.6, observed 545.6 [M-H]⁺. ³¹ P NMR (202 MHz, CDCl₃): δ −2.2 (s)

Compound 7B: ¹H NMR (500 MHz, CDCl₃): δ8.45 (1H, s), 7.83 (1H, d, J8.0 Hz), 7.29-7.20 (1H, m), 7.20-7.13 (3H, m), 6.35 (1H, d, J18.5 Hz), 5.82 (1H, d, J7.0 Hz), 4.60-4.40 (4H, m), 4.32-4.15 (2H, m), 3.32-3.23 (2H, m), 1.45 (3H, d, J20.0 Hz), 1.30 (9H, s). ESI MS for C₂₂H₂₈FN₂O₇PS₂ calculated 546.6, observed 545.5 [M-H]⁺. ³¹P NMR (202 MHz, CDCl₃) δ1.1 (s)

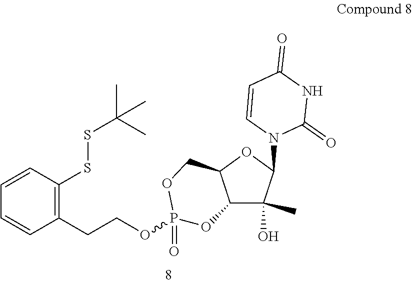

A solution of bis-(N,N-diisopropylamino)-chlorophosphine (0.27 g, 1.0 mmol) in dry dichloromethane (0.5 mL) was added dropwise to a solution of 2-Me-2′-OH-uridine (0.26 g, 1.0 mmol) and N,N-diisopropylethylamine (0.19 mL, 1.1 mmol) in dry N,N-dimethylformamide (2.0 mL) at −78° C. The reaction mixture warmed to room temperature and was stirred for 1 hour. A solution of 4,5 dicyanoimidazole (0.11 g, 1.0 mmol) in dry N,N-dimethylformamide (0.5 mL) was added and the resulting mixture was stirred for an additional 1 hour. The solution of the alcohol 1 (0.24 g, 1.0 mmol) and 4,5-dicyanoimidazole (0.11 g, 1.0 mmol) in dry N,N-dimethylformamide (1.0 mL) was added and the resulting mixture was stirred overnight. A solution of t-butyl hydroperoxide (0.2 mL, 5-6 M in decane) was added and the mixture was stirred for 30 min. The volatiles were removed in vacuo to afford a residue, which was subjected to HPLC purification (acetonitrile/H₂O; 20%-60%, 30 min) to give 0.023 g of the more polar isomer 8A and 0.012 g of the less polar isomer 8B as white powders (7%).

Compound 8A: ¹H NMR (500 MHz, CD₃OD): δ7.86 (1H, dd, J8.0, 1.0 Hz), 7.38 (1H, d, J7.5 Hz), 7.33-7.28 (2H, m), 7.24 (1H, td, J7.0, 1.0 Hz), 6.04 (1H, s), 5.84 (1H, d, J7.0 Hz), 4.59 (1H, d, J23.0 Hz), 4.41 (1H, t, J6.5 Hz), 4.40 (1H, t, J 6.5 Hz), 4.28-4.19 (2H, m), 3.83 (1H, d, J7.5 Hz), 3.33 (2H, t, J6.5 Hz), 1.29 (9H, s), 1.16 (3H, s). ESI MS for C₂₂H₂₉N₂O₈PS₂ calculated 544.6, observed 543.6 [M-H]⁺. ³¹ P NMR (202 MHz, CD₃OD) δ −0.46 (s)

Compound 8B: ¹H NMR (500 MHz, CD₃OD): δ7.84 (1H, dd, J8.0, 1.0 Hz), 7.58 (1H, d, J8.0 Hz), 7.29-7.25 (2H, m), 7.21 (1H, m), 6.09 (1H, s), 5.74 (1H, d, J8.0 Hz), 4.59-4.50 (2H, m), 4.43-4.35 (3H, m), 4.30 (1H, d, J10.0 Hz), 3.33 (2H, t, J6.5 Hz), 1.29 (9H, s), 1.26 (3H, s). ESI MS for C₂₂H₂₉N₂O₈PS₂ calculated 544.6, observed 543.6 [M-H]⁺. ³¹P NMR (202 MHz, CD₃OD) δ1.2 (s)

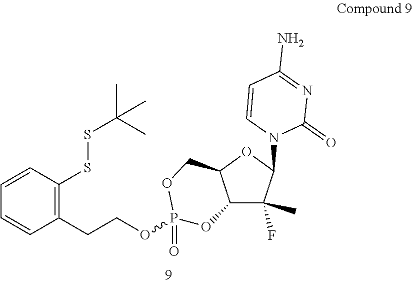

A solution of bis-(N,N-diisopropylamino)-chlorophosphine (0.27 g, 1.0 mmol) in dry dichloromethane (0.5 mL) was added dropwise to a solution of 2-Me-2′-F-cytidine (0.26 g, 1.0 mmol) and N,N-diisopropylethylamine (0.19 mL, 1.1 mmol) in dry N,N-dimethylformamide (2.0 mL) at −78° C. The reaction mixture warmed to room temperature and was stirred for 1 hour. A solution of 4,5 dicyanoimidazole (0.11 g, 1.0 mmol) in dry N,N-dimethylformamide (0.5 mL) was added, and the resulting mixture was stirred for an additional 1 hour. The solution of the alcohol 1 (0.24 g, 1.0 mmol) and 4,5-dicyanoimidazole (0.11 g, 1.0 mmol) in dry N,N-dimethylformamide (1.0 mL) was added, and the resulting mixture was stirred overnight. A solution of t-butyl hydroperoxide (0.2 mL, 5-6 M in decane) was added and the mixture was stirred for 30 min. The volatiles were removed in vacuo to afford a residue, which was subjected to HPLC purification (acetonitrile/H₂O; 20%-60%, 30 min) to give 0.023 g (47%) of the more polar isomer 9A and 0.017 g of the less polar isomer 9B as white powders (7%).

Compound 9A: ¹H NMR (500 MHz, CD₃OD): δ7.86 (1H, dd, J8.0, 1.0 Hz), 7.73 (1H, m), 7.32-7.28 (2H, m), 7.23 (1H, td, J7.0, 1.0 Hz), 6.30 (1H, d, J18.0 Hz), 6.20 (1H, s, br), 4.64 (1H, s, br), 4.44-4.25 (2H, m), 4.44 (1H, t, J7.0 Hz), 4.43 (1H, t, J7.0 Hz), 4.09-4.01 (1H, m), 3.35 (2H, t, J7.0 Hz), 1.40 (3H, d, J20.0 Hz), 1.30 (9H, s). ESI MS for C₂₂H₂₉FN₃O₆PS₂ calculated 545.6, observed 544.4 [M-H]⁺. ³¹P NMR (202 MHz, CD₃OD) δ1.09 (s)

Compound 9B: ¹H NMR (500 MHz, CD₃OD): δ7.85 (1H, d, J1.0 Hz), 7.84 (1H, s), 7.30-7.25 (2H, m), 7.21 (1H, td, J7.0, 1.0 Hz), 6.31 (1H, d, J18.0 Hz), 6.13 (1H, d, J8.0 Hz), 4.60 (1H, s, br), 4.50-4.30 (2H, m), 4.43 (1H, t, J7.0 Hz), 4.40 (1H, t, J7.0 Hz), 3.31 (2H, t, J7.0 Hz), 1.45 (3H, d, J20.0 Hz), 1.30 (9H, s). ESI MS for C₂₂H₂₉FN₃O₆PS₂ calculated 545.6, observed 544.5 [M-H]⁺. ³¹P NMR (202 MHz, CD₃OD) δ0.63(s)

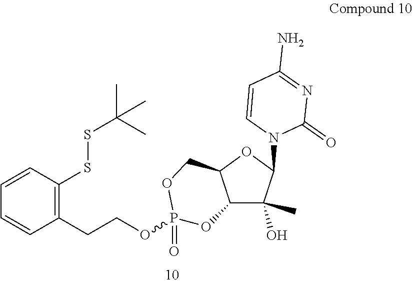

A solution of bis-(N,N-diisopropylamino)-chlorophosphine (0.27 g, 1.0 mmol) in dry dichloromethane (0.5 mL) was added dropwise to a solution of 2-Me-2′-OH-cytidine (0.26 g, 1.0 mmol) and N,N-diisopropylethylamine (0.19 mL, 1.1 mmol) in dry N,N-dimethylformamide (2.0 mL) at −78° C. The reaction mixture warmed to room temperature and was stirred for 1 hour. A solution of 4,5-dicyanoimidazole (0.11 g, 1.0 mmol) in dry N,N-dimethylformamide (0.5 mL) was added and the resulting mixture was stirred for an additional 1 hour. The solution of the alcohol 1 (0.24 g, 1.0 mmol) and 4,5-dicyanoimidazole (0.11 g, 1.0 mmol) in dry N,N-dimethylformamide (1.0 mL) was added and the resulting mixture was stirred overnight. A solution of t-butyl hydroperoxide (0.2 mL, 5-6 M in decane) was added and the mixture was stirred for 30 min. The volatiles were removed in vacuo to afford a residue, which was subjected to HPLC purification (acetonitrile/H₂O; 20%-60%, 30 min) to give 0.042 g (47%) of the more polar isomer 10A and 0.007 g of the less polar isomer 10B as white powders (9%).

Compound 10A: ¹H NMR (500 MHz, CD₃OD): δ7.86 (1H, dd, J8.0 , 1.0 Hz), 7.70 (1H, d, J6.5 Hz), 7.32-7.28 (2H, m), 7.23 (1H, td, J7.0, 1.0 Hz), 6.20 (1H, d, J7.0 Hz), 6.05 (1H, s), 4.62 (1H, d, J22.0 Hz), 4.41 (1H, t, J6.5 Hz), 4.40 (1H, t, J6.5 Hz), 4.35-4.25 (2H, m), 3.86 (1H, s), 3.34 (2H, t, J6.5 Hz), 1.29 (9H, s), 1.18 (3H, s); ESI MS for C₂₂H₃₀N₃O₇PS₂ calculated 543.6, observed 542.5 [M-H]⁺; ³¹ P NMR (202 MHz, CD₃OD) δ-0.50 (s)

Compound 10B: ¹H NMR (500 MHz, CD₃OD): δ7.84 (1H, dd, J8.0, 1.0 Hz), 7.84 (1H, s), 7.30-7.27 (2H, m), 7.21 (1H, td, J7.5, 1.0 Hz), 6.15-6.07 (2H, m), 4.60-4.52 (2H, m), 4.48-4.39 (3H, m), 4.26 (1H, d, J9.0 Hz), 3.34 (2H, t, J6.5 Hz), 1.30 (9H, s), 1.26 (3H, s); ESI MS for C₂₂H₃₀N₃O₇PS₂ calculated 543.6, observed 542.9 [M-H]⁺; ³¹P NMR (202 MHz, CD₃OD) δ1.25 (s)

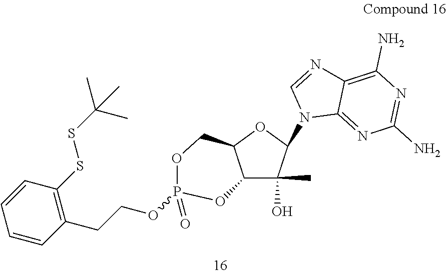

### Synthesis of Compound 13:

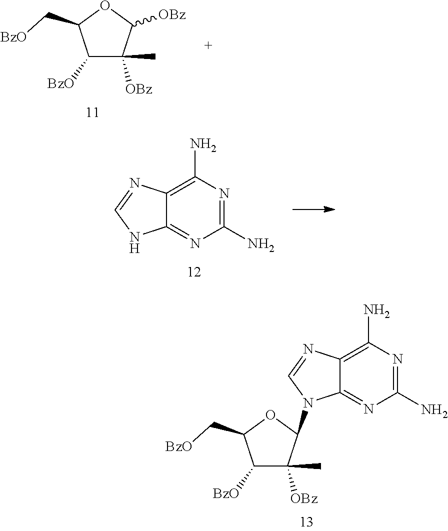

To a stirred suspension of (3R,4S,5R)-5-((benzoyloxy)methyl)-3-methyltetrahydrofuran-2,3,4-trityl tribenzoate 11 (2.9 g, 5 mmol) and 2,6-diaminopurine 12 (0.83 g, 5.5 mmol) in anhydrous acetonitrile (30 mL) at −78° C. was added DBU (2.3 mL, 15.0 mmol), followed by a slow addition of TMSOTf (3.8 mL, 20.0 mmol). The reaction mixture was heated to 65° C. and stirred overnight, cooled to room temperature, and diluted with dichloromethane (200 mL). The resulting mixture was washed with saturated aq. NaHCO₃. The organic layer was separated, and the aqueous layer was extracted with dichloromethane (2×20 mL). The combined organic layers were dried over anhydrous sodium sulfate. Volatiles were removed in vacuo to afford a residue, which was subjected to flash silica gel column purification on an ISCO companion (2-10% methanol/ethyl acetate) to give 1.5 g (49%) of compound 13 as a white solid. ¹H NMR (500 MHz, CDCl₃): δ8.15-8.17 (2H, m), 7.97-8.02 (4H, m), 7.77 (1H, s), 7.45-7.61 (5H, m), 7.32-7.37 (4H, m), 6.62 (1H, s), 6.53 (1H, d, J6.5 Hz), 5.91 (2H, s), 5.04-5.10 (3H, m), 4.82-4.86 (1H, m), 4.70-4.74 (1H, m), 1.62 (3H, s); ESI MS for C₃₂H₂₈N₆O₇ calculated 608.6, observed 609.2 [M+H]⁺

### Synthesis of Compound 14:

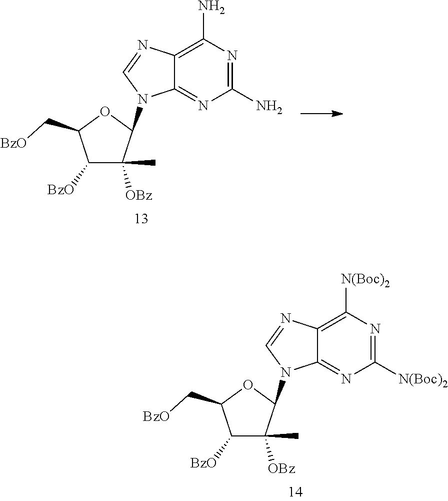

To a solution of 13 (1.0 g, 1.64 mmol) in THF (10 mL) were added Boc anhydride (2.15 g, 9.86 mmol) and DMAP (0.040 g, 0.33 mmol), and the mixture was stirred for 24 hrs. Volatiles were removed in vacuo to afford a residue, which was subjected to flash silica gel column purification on an ISCO companion (0-40% ethyl acetate/hexane) to give 1.2 g (73%) of compound 14 as a white solid. ¹H NMR (500 Hz, CDCl₃): δ8.37 (1H, s), 8.17 (2H, d, J7.5 Hz), 8.07 (2H, d, J7.5 Hz), 7.87 (2H, d, J7.5 Hz), 7.61-7.55 (2H, m), 7.52-7.47 (3H,), 7.43 (2H, t, J7.5 Hz), 7.27 (2H, t, J7.5 Hz), 6.74 (1H, s), 6.00 (1H, d, J5.0 Hz), 4.97-4.91 (2H, m), 4.73 (1H, q, J5.0 Hz), 1.58 (3H, s), 1.43 (18H, s), 1.37 (18H, s)

### Synthesis of Compound 15:

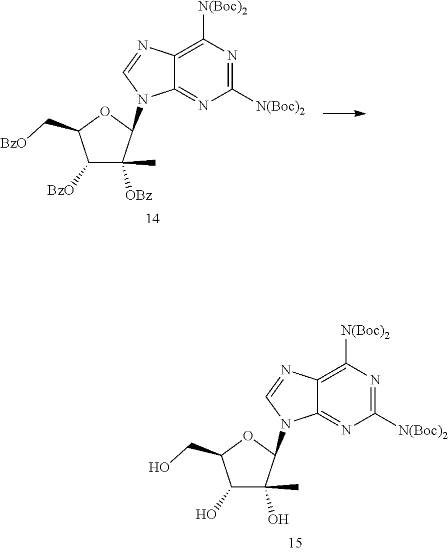

To a solution of 14 (1.15 g, 1.14 mmol) in methanol (40 mL) was added a solution of sodium methoxide (4.37 M, 0.23 mL, 1.0 mmol), and the mixture was stirred for 30 min. The reaction mixture was neutralized by portionwise addition of Dowex® resin (H⁺form) to pH=7.0, and the resin was filtered, and washed with methanol. The filtrate was evaporated to give a residue, which was subjected to flash silica gel column purification on an ISCO companion (30-100% ethyl acetate/hexane) to give 0.65 g (73%) of compound 15 as a white solid. ¹H NMR (400 MHz, CD₃OD): δ9.09 (1H, s), 6.19 (1H, s), 4.22 (1H, d, J8.8 Hz), 4.03-4.11 (2H, m), 3.89 (1H, dd, J12.5, 3.0 Hz), 1.41 (18H, s), 0.92 (3H, s); ESI MS for C₃₁H₄₈N₆O₁₂ calculated 696.7, observed 697.4 [M+H]⁺

### Synthesis of Compound 16:

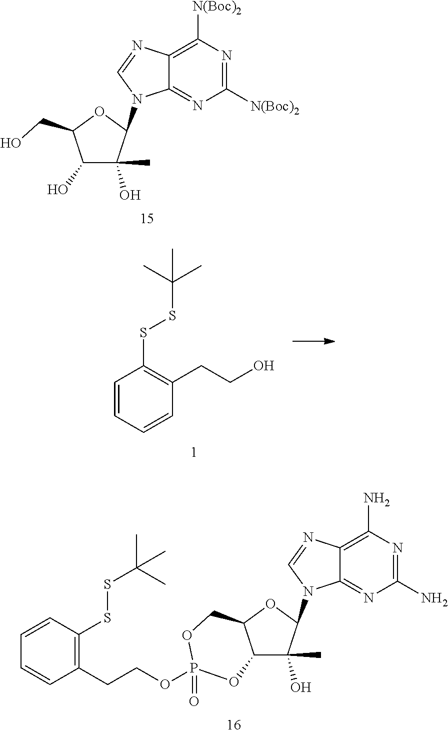

A solution of bis-(N,N-diisopropylamino)-chlorophosphine (0.25 g, 0.94 mmol) in dry dichloromethane (2.0 mL) was added dropwise to a solution of 15 (0.65 g, 0.94 mmol) and N,N-diisopropylethylamine (0.176 mL, 1.03 mmol) in dry dichloromethane (5.0 mL) at −78° C. The reaction mixture was warmed to room temperature and stirred for 1 hour. A solution of 4,5-dicyanoimidazole (0.11 g, 0.94 mmol) in dry acetonitrile (3 mL) was added, and the resulting mixture was stirred for 1 hour. To this, a solution of the alcohol 1 (0.23 g, 0.94 mmol) and 4,5-dicyanoimidazole (0.11 g, 0.94 mmol) in acetonitrile (5 mL) was added, and the resulting mixture was stirred overnight. t-Butyl hydroperoxide solution (0.19 mL, 5-6 M in decane) was added, and the mixture was stirred for additional 30 min. Volatiles were 20 removed in vacuo to afford a residue, which was treated with 4 mL of TFA/DCM (1:1) mixture. The resulting mixture was stirred for 2 hrs. Volatiles were removed in vacuo to afford a residue, which was subjected to HPLC purification (acetonitrile/H₂O; 20%-55%, 30 min) to give 0.022 g of the more polar diastereomer 16A and 0.008 g of the less polar diastereomer 16B as white solids.

Compound 16A: ¹H NMR (500 MHz, CD₃OD): δ7.87 (1H, dd, J8.0, 1.0 Hz), 7.84 (1H, s), 7.31 (1H, d, J7.5 Hz), 7.28 (1H, td, J7.5, 1.0 Hz), 7.22 (1H, td, J7.5, 1.0 Hz), 5.97 (1H, s), 4.66-4.60 (1H, m), 4.48-4.32 (5H, m), 3.36 (2H, t, J6.5 Hz), 1.27 (9H, s), 1.00 (3H, s); ESI MS for C₂₃H₃₁N₆O₆PS₂ calculated 582.6, observed 581.6 [M-H]⁺; ³¹P NMR (202 MHz, CD₃OD) δ-0.28 (s)

Compound 16B: ¹H NMR (500 MHz, CD₃OD): δ7.86 (1H, d, J8.0 Hz), 7.77 (1H, s), 7.32 (1H, d, J7.5 Hz), 7.28 (1H, td, J7.5, 1.0 Hz), 7.21 (1H, td, J7.5, 1.0 Hz), 5.95 (1H, s), 4.66-4.58 (1H, m), 4.48-4.32 (5H, m), 3.36 (2H, t, J7.0 Hz), 1.27 (9H, s), 1.00 (3H, s); ESI MS for C₂₃H₃₁N₆O₆PS₂ calculated 582.6, observed 581.5 [M-H]⁺; ³¹P NMR (202 MHz, CD₃OD) δ2.0(s)

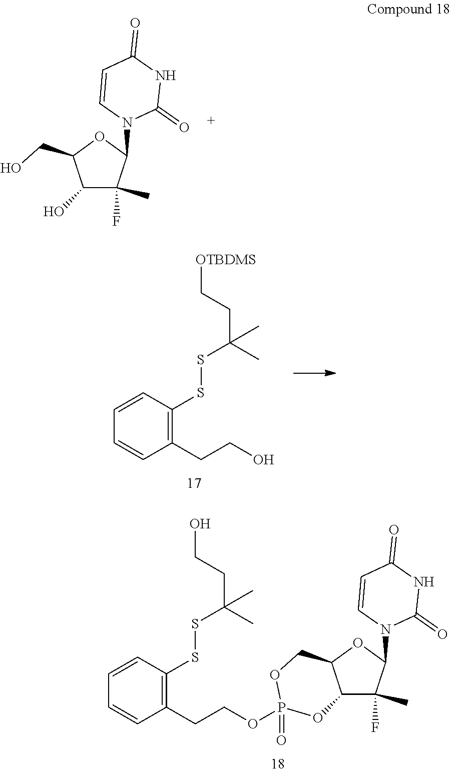

Two diastereomers of compound 18 were synthesized using the same procedure reported for compound 7 employing TBDMS protected disulfide 17 followed by deprotection using TBAF in THF. Compound 18A: ESI MS for C₂₃H₃₀FN₂O₈PS₂ calculated 576.6, observed 577.5 [M+H]⁺; Compound 18B: ESI MS for C₂₃H₃₀FN₂O₈PS₂ calculated 576.6, observed 577.3 [M+H]⁺.

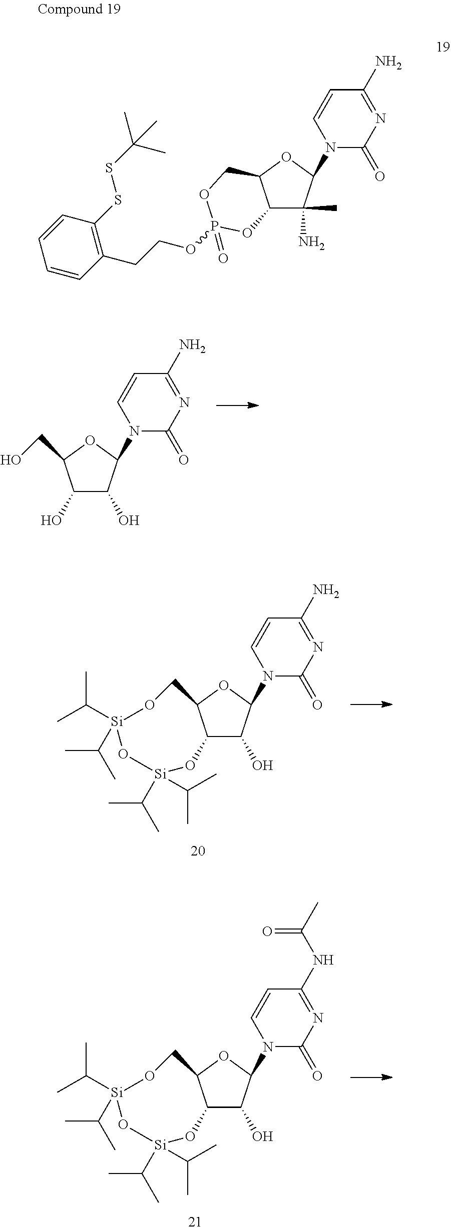

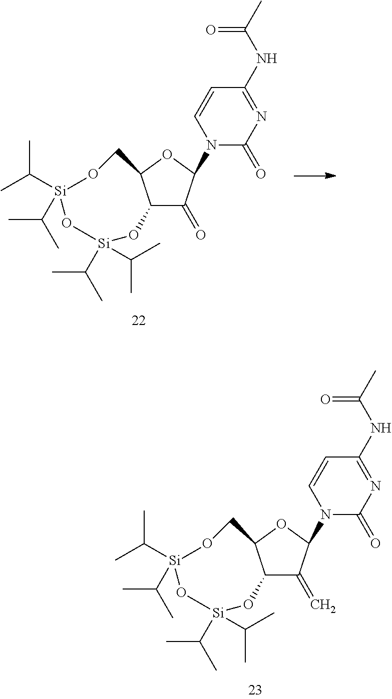

### Synthesis of Compound 20

Cytidine (10.0 g, 41.1 mmol) was azeotroped with pyridine (2×20 mL) and suspended in 40.0 mL of pyridine. To the suspension was added tetraisopropyldisiloxanedichloride (14.3 g, 45.2 mmol) dropwise over 15 minutes. The resulting suspension was stirred for about 16 hours at room temperature. The reaction mixture was carefully diluted with water and extracted with ethyl acetate. The organic layer was washed with brine, dried over anhydrous Na₂SO₄, filtered, and concentrated in vacuo. The residue was triturated with hexane to provide compound 20 as a white solid. ESI MS for C₂₁H₃₉N₃O₆Si₂ calculated 485.7, observed 486.2 [M+H]⁺

### Synthesis of Compound 21

Compound 20 was dissolved in 200 mL of ethanol and treated with 20.0 mL of acetic anhydride. The reaction mixture was heated to reflux and stirred for 3 hours. The solvent was removed under reduced pressure. The residue obtained was cooled to 0° C. in an ice bath, treated with saturated NaHCO₃, and extracted with ethyl acetate. The organic layer was washed with brine, dried over anhydrous Na₂SO₄, filtered, and concentrated in vacuo. The crude mixture was purified by silica gel column chromatography using ethyl acetate/hexane solvent system (0-70% gradient on Combi Flash Rf Instrument) to give 9.0 g of product 21 as a white solid (42% over two steps). ESI MS for C₂₃H₄₁N₃O₇Si₂ calculated 527.7, observed 528.3 [M+H]⁺

### Synthesis of Compound 22

A solution of compound 21 (8.6 g, 16.3 mmol) in 200 mL of dichloromethane was cooled to 0° C. in an ice bath and treated with Dess-Martin periodinane (17.4 g, 40.8 mmol). The resulting mixture was stirred for about 16 hours at room temperature and diluted with diethyl ether. The solution was washed with a mixture of saturated NaHCO₃ and 10% sodium thiosulfate (v/v=1:1). The organic layer was dried over anhydrous Na₂SO₄, filtered, and concentrated in vacuo. The crude mixture was purified by silica gel column chromatography using ethyl acetate/hexane solvent system (0-60% gradient on Combi Flash Rf Instrument) to give 6.0 g of product 22 as a light yellow foam (72%).

ESI MS for C₂₃H₃₉N₃O₇Si₂calculated 525.7, observed 526.2 [M+H]⁺

### Synthesis of Compound 23

To the suspension of methyltriphenylphosphonium bromide (16.2 g, 45.4 mmol) in 150 mL of tetrahydrofuran was added KHMDS solution (0.5M in toluene, 87.0 mL, 43.3 mmol) dropwise under argon. The reaction mixture was allowed to stir at room temperature for 30 minutes, cooled to 0° C. in an ice bath, and treated with a solution of compound 22 (6.0 g, 11.4 mmol) in 40.0 mL of tetrahydrofuran dropwise. The resulting mixture was warmed to room temperature and stirred for 4 hours. The reaction mixture was quenched with saturated ammonium chloride and extracted with ethyl acetate. The organic layer was washed with brine, dried over anhydrous Na₂SO₄, filtered and concentrated in vacuo. The crude mixture was purified by silica gel column chromatography using ethyl acetate/hexane solvent system (0-50% gradient on Combi Flash Rf Instrument) to give 4.7 g of compound 23 as a white foam (79%). ¹H NMR (500 MHz, CDCl₃): δ9.96 (1H, s), 8.0 (1H, d, J7.5 Hz), 7.43 (1H, d, J7.5 Hz), 6.61 (1H, d, J1.0 Hz), 5.71 (1H, d, J1.5 Hz), 5.39 (1H, t, J2.0 Hz), 4.81 (1H, dd, J9.0, 1.0 Hz), 4.2 (1H, dd, J13.5, 1.5 Hz), 4.05 (1H, dd, J13.0, 2.5 Hz), 3.73 (1H, dd, J9.0, 4.5 Hz), 2.27 (s, 3H), 1.12-1.02 (m, 28H); ESI MS for C₂₄H₄₁ N₃O₆Si₂ calculated 523.7, observed 524.2 [M+H]⁺

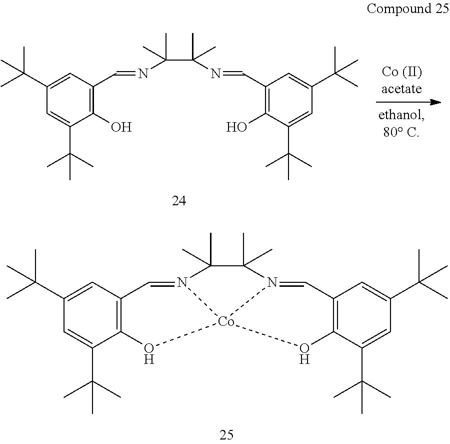

N,N-Bis(3,5-di-tert-butylsalicylidene)-1,1,2,2-tetramethylethylenediamine (24, 0.73 g, 1.3 mmol) was suspended in 10.0 mL of ethanol. The resulting suspension was heated to 80° C. and stirred for 5 minutes under argon balloon. Cobalt (II) acetate (0.24 g, 1.3 mmol) was then added, and the reaction mixture was stirred for another 2 hours at 80° C. The crimson red suspension was cooled down to room temperature in an ice bath and was filtered. The collected red solid was dried under vacuum to provide 0.70 g of compound 25 (87%).

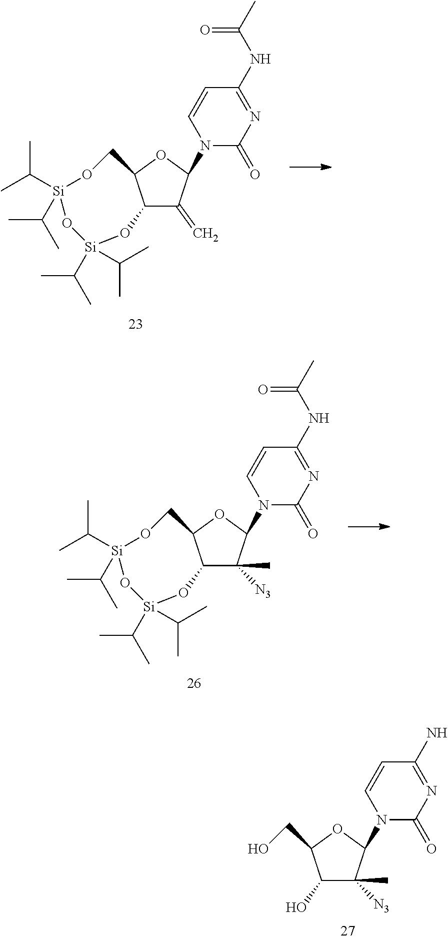

### Synthesis of Compound 26

Compound 23 (4.7 g, 9.0 mmol) and compound 25 (0.19 g, 0.3 mmol) were dissolved in 4-methylbenzenesulfonyl azide (28.4 g, 144 mmol), and the reaction mixture was stirred for 5 minutes at room temperature. A solution of phenylsilane (1.17 g, 10.8 mmol) in 30.0 mL of ethanol was added dropwise, and the reaction mixture was allowed to stir for an additional 40 minutes. The reaction was quenched with brine and extracted with ethyl acetate. The organic layer was washed with brine, dried over anhydrous Na₂SO₄, filtered, and concentrated in vacuo. The crude mixture was purified by silica gel column chromatography using ethyl acetate/hexane solvent system (0-30% gradient on Combi Flash Rf Instrument) to give 3.34 g of product 26 as a light yellow solid (66%). ¹H NMR (500 MHz, CDCl₃): δ9.78 (1H, br), 8.18 (1H, d, J7.5 Hz), 7.43 (1H, d, J7.5 Hz), 5.9 (1H, s), 4.24 (1H, d, J13.5 Hz), 4.17-4.12 (m, 2H), 4.04 (1H, d, J13.5 Hz), 2.27 (s, 3H), 1.40 (s, 3H), 1.12-1.02 (m, 28H); ESI MS for C₂₄H₄₂N₆O₆Si₂ calculated 566.8, observed 567.3 [M+H]⁺

### Synthesis of Compound 27

Compound 26 (3.34 g, 5.9 mmol) was dissolved in 25.0 mL of tetrahydrofuran and treated with a solution of tetrabutylammonium fluoride (1.0M in THF, 11.8 mL, 11.8 mmol). The reaction mixture was stirred for 1 hour at room temperature and concentrated in vacuo. The residue obtained was dissolved in a mixture of 30% aqueous ammonia (15.0 mL) and methylamine (15.0 mL), stirred for 3 hours, and condensed in vacuo. The crude mixture was purified by silica gel column chromatography using methanol/dichloromethane system (0-20% gradient on Combi Flash Rf Instrument) to give 1.2 g of product 27 as a white solid (72%). ¹H NMR (500 MHz, CD₃OD): δ8.56 (1H, d, J8.0 Hz), 6.09 (1H, d, J7.5 Hz), 5.86 (1H, s), 4.09 (1H, d, J9.5 Hz), 4.01-3.96 (2H, m), 3.80 (1H, d, J13.0 Hz), 1.39 (3H, s); ESI MS for C₁₀H₁₄N₆O₄calculated 282.2, observed 283.5 [M+H]⁺

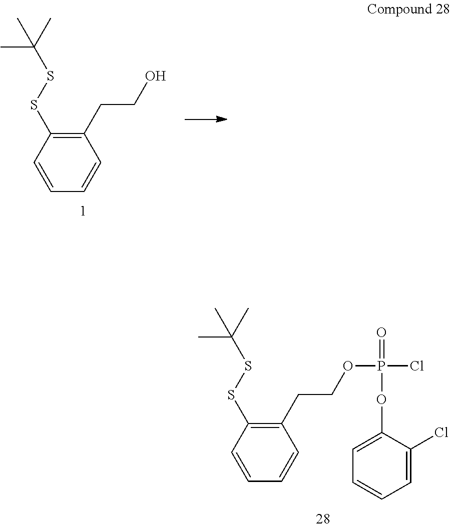 [SMILES: CC(C)(C)SSc1ccccc1CCO ; CC(C)(C)SSc1ccccc1CCOP(=O)(Cl)Oc1ccccc1Cl]

A solution of 2-chlorophenyl phosphorodichloridate (2.0 g, 8.3 mmol) in 10.0 mL of anhydrous THF (over 4 Å molecular sieves) was cooled in an ice bath and was added compound 1 (2.0 g, 8.3 mmol) in 5.0 mL of THF under argon, followed by the dropwise addition of 2,6-lutidine (0.89 g, 8.3 mmol). The reaction mixture was allowed to warm to room temperature and stirred for another 3 hours. The suspension was filtered and the filtrate was concentrated in vacuo, and the residue was purified by silica gel column chromatography using ethyl acetate/hexane solvent system (0-30% gradient on Combi Flash Rf Instrument) to give 2.5 g of product 28 as a colorless oil (68%).

¹H NMR (500 MHz, CDCl₃): δ7.81 (1H, d, J8.0 Hz), 7.37 (1H, d, J8.0 Hz), 7.29-7.07 (6H, m), 4.41 (2H, t, J7.0 Hz), 4.12 (2H, t, J7.0 Hz), 1.27 (9H, s)

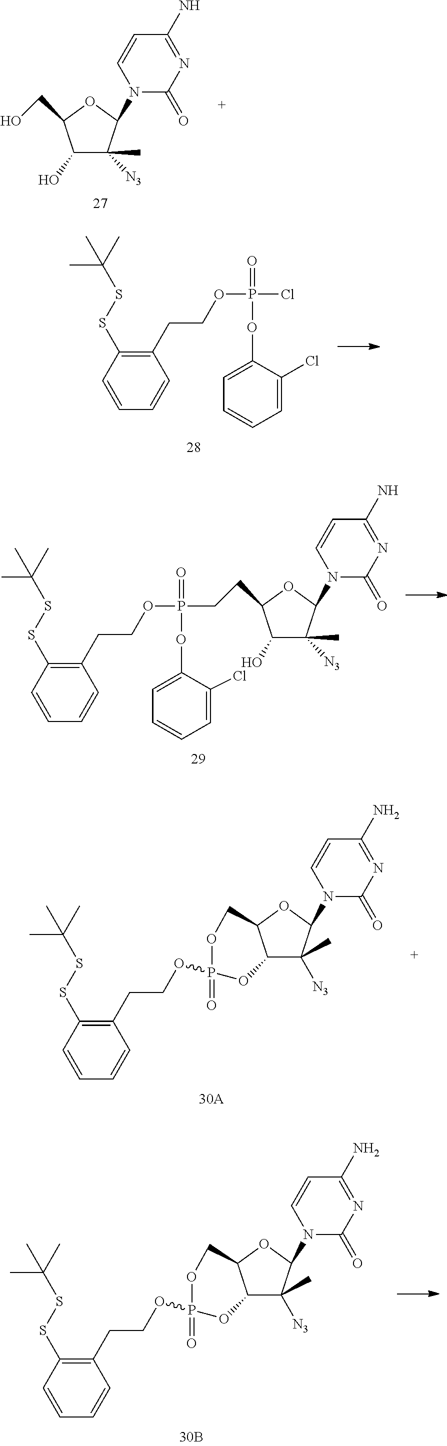

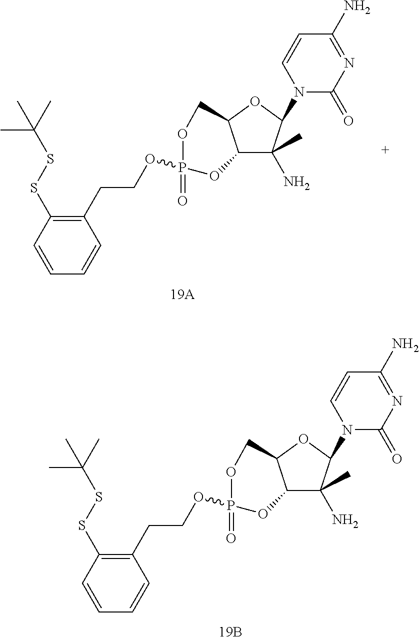

### Synthesis of Compound 29

A solution of compound 27 (0.10 g, 0.35 mmol) in 1.0 mL of anhydrous THF (over 4 Å molecular sieves) was cooled in an ice bath and was added 1.0 mL of 1-methylimidazole. The reaction mixture was stirred for 15 minutes until the clear reaction solution was formed, followed by the dropwise addition of a solution of compound 28 (0.17 g, 0.39 mmol) in 1.0 mL of THF. The reaction mixture was allowed to warm to room temperature, stirred for additional 2 hours, and the reaction was quenched with water and extracted with ethyl acetate. The organic layer was washed with saturated ammonium chloride and brine, dried over anhydrous Na₂SO₄, filtered, and concentrated in vacuo. The residue was purified by silica gel column chromatography using methanol/dichloromethane solvent system (0-15% gradient on Combi Flash Rf Instrument) to give 0.050 g of product 29 as a colorless oil (20%). ESI MS for C₂₈H₃₄ClN₆O₇PS₂ calculated 697.1, observed 697.8 [M+H]⁺

### Synthesis of Compound 30

A solution of compound 29 (0.025 g, 0.035 mmol) in 1.0 mL of anhydrous THF (over 4 Å molecular sieves) was cooled in an ice bath and was added potassium t-butoxide (0.008 g, 0.071 mmol) in one portion. The reaction mixture was allowed to warm to room temperature and stirred for another 15 minutes. The reaction was quenched with saturated ammonium chloride at 0° C. and concentrated under reduced pressure. The residue was diluted with ethyl acetate, washed with brine, dried over anhydrous Na₂SO₄, filtered, and concentrated in vacuo. The crude mixture of diastereoisomers 30A and 30B was used in the next step without purification. ESI MS for C₂H₂₉N₆O₆PS₂calculated 568.6, observed 569.4 [M+H]⁺

### Synthesis of Compound 19

To a solution of compounds 30A and 30B in 1.0 mL mixture of THF/water (4:1, v/v) was added triphenylphosphine (0.009 g, 0.035 mmol), and the reaction mixture was stirred for 16 hours at room temperature. The solvent was removed under reduced pressure, and the residue was diluted with methanol and purified by preparative HPLC (C18 column, acetonitrile/H₂O/0.1%TFA) to give 0.004 g of product 19A (more polar) and 0.001 g of product 19B (less polar) as white solids.

Compound 19A: ¹H NMR (500 MHz, CD₃OD): δ7.87 (1H, d, J7.5 Hz), 7.73 (1H, m), 7.33-7.23 (3H, m), 6.15-6.13 (2H, m), 4.72 (1H, dd, J23, 4.5 Hz), 4.47 (2H, m), 4.32 (2H, m), 4.21 (1H, m), 3.36 (2H, m), 1.30 (9H, s), 1.25 (3H, s); ESI MS for C₂₂H₃₁N₄O₆PS₂calculated 542.6, observed 543.2 [M+H]⁺; ³¹P NMR (202 MHz, CDCl₃) δ −7.07 (s)

Compound 19B: ¹H NMR (500 MHz, CD₃OD): δ7.85 (1H, d, J7.5 Hz), 7.70 (1H, m), 7.30-7.20 (3H, m), 6.19 (1H, m), 6.03 (1H, m), 4.68-4.65 (3H, m), 4.48-4.42 (3H, m), 3.31 (2H, m), 1.33 (3H, s), 1.30 (9H, 5); ³¹P NMR (202 MHz, CDCl₃) δ −4.38 (s)

### Compound 31

Cytidine pharmacophores are known to be metabolized to uridines via a deamination process. This conversion can compromise the pharmacological outcome of the cytidine pharmacophores. In one embodiment, this metabolic liability may be reduced by employing a heavy atom approach that slows or even stops metabolic activity, e.g., insertion of ¹⁵N into to the cytidine base.

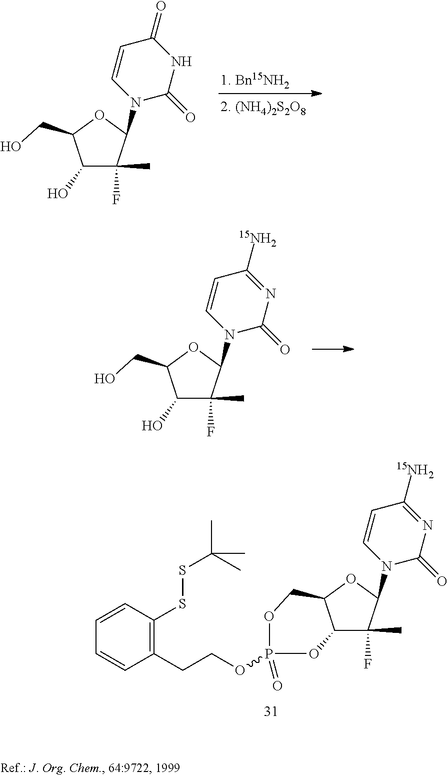

Compound 31 can be prepared using procedure similar to the preparation described for compound 7.

The following compounds can be prepared according to methods described herein:

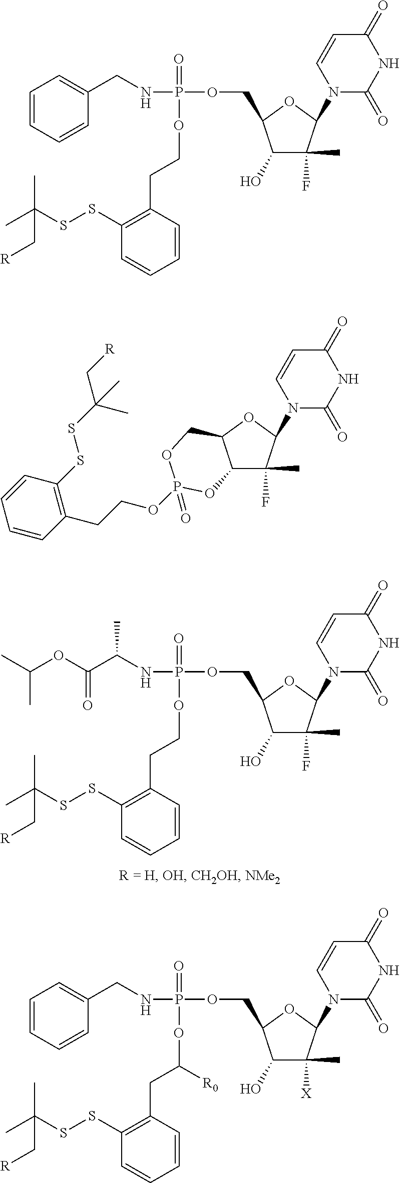

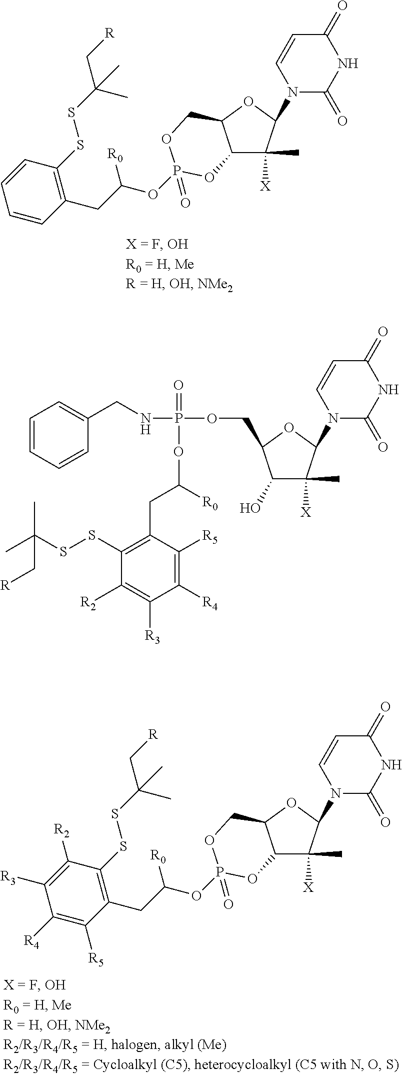

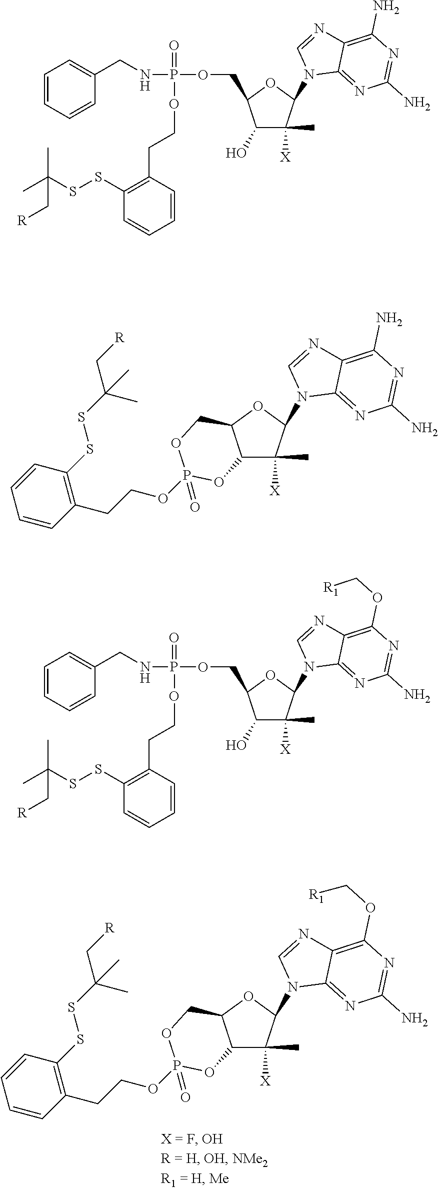

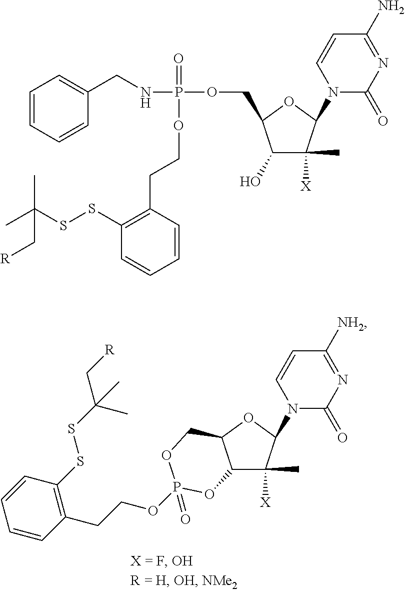

or a pharmaceutically acceptable salt or a phosphorus diastereomer thereof.

### Conjugates

### Synthesis of Cell Penetrating Peptides (Protein Transduction Domains)

Peptide Synthesis:

Synthesis: Rink amide polystyrene resin (0.080 g, 0.61 mmol/g) was added to the reaction vessel, swelled three times in dimethylformamide (5 volumes) for 7 min. each time with nitrogen bubbling and then drained. The assembly of the peptide was carried out using the following cycles and employing standard Fmoc chemistry:
Fmoc deprotection with 20% piperidine in dimethylformamide (DMF) 3×4 min;
Resin washed with DMF, 6×1 min;
Couplings used 5 eq. protected amino acid, 15 eq. N-methylmorpholine (NMM), and 5 eq. HCTU. After adding the coupling solution, the reaction was allowed to proceed for 2×20 min;
On completion of coupling, the resin was washed with DMF for 6×1 min;
For the final assembly step, the N-terminus was capped by adding 5 eq. of Fmoc-6-Hydrazinoicotinic Acid; 5 eq. HATU and 15 eq. NMM in DMF and mixing until the reaction was complete (around 1 hr), as confirmed by the Kaiser (ninhydrin) test. The Fmoc removed by 20% piperidine in DMF 3×4 min; and
The completed resin-bound peptide was washed three times with DMF, three times with dichloromethane (DCM) and then dried under vacuum.

Cleavage: The peptide was cleaved/deprotected from the resin using the following solution: trifluoroacetic acid/dithiothreitol/water/acetone/triisopropylsilane (10 ml, 90/3/2/3/2), with stirring for 2 hr. The resin was filtered through a medium frit, syringe filter and washed twice with neat trifluoroacetic acid (TFA). The filtrates were combined and the volume reduced to half by evaporation. The TFA solution was stirred and the crude peptide precipitated by the slow addition of 4 volumes of ice-cold ether. The precipitated crude peptide was collected by filtration.

Purification: The crude material was analyzed by LC/MS using a 15-75% B (A=0.1% trifluoroacetic acid/water; B=0.1% trifluoroacetic acid/acetonitrile) over 20 min using a Phenomenex Luna C₁₈ (100×4.6 mm 5 μ) column. The prepared cell penetrating peptides are listed in Table 1.

### Synthesis of Targeting Moieties

GaINAc (NAG) Ligand Synthesis:

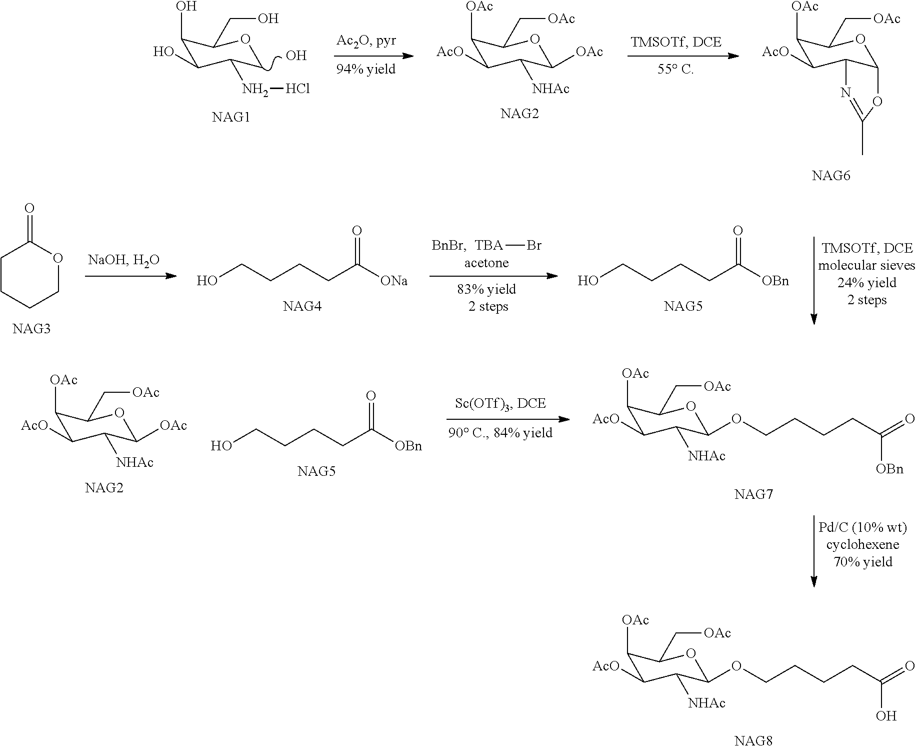

Preparation of D-galactosamine pentaacetate (NAG2). D-Galactosamine (25.0 g, 116 mmol) was suspended in anhydrous pyridine (250 mL) and cooled to 0° C. under an inert atmosphere. Acetic anhydride (120 mL, 1160 mmol) was added over the course of 2 h. After stirring overnight, the reaction mixture was concentrated in vacuo. Upon addition of methanol, a white solid precipitated and was collected via filtration to provide the desired product (42.1 g, 93% yield). ¹H NMR (CDCl₃, 500 MHz): δ 5.69 (d, 1H, J9.0 Hz), 5.40 (m, 1H), 5.37 (d, 1H, J3.0 Hz), 5.08 (dd, 1H, J3.0 Hz, 11 Hz), 4.44 (dt, 1H, J9.5 Hz, 11 Hz), 4.17 (dd, 1H, J7.0 Hz, 11.5 Hz), 4.11 (dd, 1H, J7.0 Hz, 11.5 Hz), 4.01 (t, 1H, J7.0 Hz), 2.17 (s, 3H), 2.13 (s, 3H), 2.05 (s, 3H), 2.02 (s, 3H), 1.94 (s, 3H), 1.57 (s, 3H).

Preparation of benzyl 5-hydroxy pentanoate (NAG5). A solution of delta-valerolactone (10.0 g, 100 mmol) and NaOH (4.00 g, 100 mmol) in water (100 mL) was stirred overnight at 70° C. The reaction mixture was cooled to rt and concentrated in vacuo to give white solid NAG4. This solid was suspended in acetone (100 mL) and refluxed overnight with benzyl bromide (20.5 g, 120 mmol) and tetrabutylammonium bromide (1.61 g, 0.50 mmol). Acetone was removed in vacuo to afford an oily residue, which was dissolved in EtOAc and washed with sat NaHCO₃ (aq.) and brine. The organic layer was dried over Na₂SO₄ and concentrated in vacuo give the oily product NAG5 (17.1 g, 82% yield). ¹H NMR (CDCl₃, 500 MHz): δ7.35 (m, 5H), 3.64 (q, 2H, J6 Hz, 11.5 Hz), 2.41 (t, 2H, J7.5 Hz), 1.75 (m, 2H), 1.60 (m, 2H), 1.44 (t, 1H, J6 Hz).

Preparation of benzyloxycarbonylbutyl 2-deoxy 2-N-acetyl -3,4,6-tri-O-acetyl-β-D-galactopyranoside (NAG7)—Method A. Under an inert atmosphere, TMSOTf (8.56 g, 38.4 mmol) was added to a solution of NAG2 (10.0 g, 25.6 mmol) in DCE (100 mL) at ambient temperature. The mixture was stirred at 55° C. for 2 h, removed from heat, and stirred overnight. The reaction mixture was poured onto ice cold sat NaHCO₃ (aq.) and extracted with CH₂Cl₂. The organic layer was dried over Na₂SO₄ and concentrated in vacuo to give syrup NAG6. A solution NAG6 in DCE (60 mL) was charged with alcohol NAG5 (8.00 g, 38.4 mmol) and molecular sieves. The mixture was placed under an inert atmosphere, treated with TMSOTf (2.85 g, 12.8 mmol), and stirred overnight at rt. The mixture was poured over ice cold sat NaHCO₃ (aq.) and extracted with CH₂Cl₂. The organic layer was dried over Na₂SO₄ and concentrated in vacuo to give syrup. This crude material was purified via SiO₂ gel chromatography to afford glycoside NAG7 (3.3 g, 24% yield). ¹H NMR (CD01₃, 500 MHz): δ7.35 (m, 5H), 5.98 (d, 1H, J7.0 Hz), 5.57 (m, 1H), 5.34 (d, 1H, J3.0 Hz), 5.25 (dd, 1H, J3.0 Hz, 11 Hz), 5.10 (s, 2H), 4.63 (d, 1H, J8.5 Hz), 4.11 (m, 2H), 3.95 (m, 1 H), 3.88 (m, 2H), 3.49 (m, 1H), 2.37 (m, 2H), 2.13 (s, 3H), 2.03 (s, 3H), 1.99 (s, 3H), 1.90 (s, 3H), 1.70 (m, 2H), 1.61 (m, 2H).

Preparation of benzyloxycarbonylbutyl 2-deoxy 2-N-acetyl -3,4,6-tri-O-acetyl-β-D-galactopyranoside (NAG7)—Method B. To a solution of NAG2 (5.00 g, 12.8 mmol) and alcohol NAG5 (5.33 g, 25.6 mmol) in DCE (50 mL) was added Sc(OTf)₃ (0.44 g, 0.90 mmol) in one portion. The mixture was placed under an inert atmosphere and refluxed for 3 h. Upon cooling the mixture was diluted with CH₂Cl₂, washed with sat NaHCO3 (aq.), dried over MgSO₄, and concentrated in vacuo. Purification via SiO₂ gel chromatography afforded glycoside NAG7 (5.53 g, 80% yield).

Preparation of carboxybutyl 2-deoxy 2-N-acetyl -3,4,6-tri-O-acetyl-β-D-galactopyranoside (NAG8). A solution of glycoside NAG7 (1.50 g, 2.41 mmol) in EtOH (25 mL) was degassed under vacuum and purged with argon. The palladium catalyst (10% wt. on activated carbon, 0.50 g) was added in one portion and the mixture was degassed under vacuum purged with argon. The heterogeneous mixture was charged with cyclohexene (25 mL) and refluxed for 6 h. Upon cooling the catalyst was removed by filtration and the mother liquor concentrated in vacuo. The crude was purified via SiO₂ gel chromatography to afford a white foam NAG8 (0.76 g, 70% yield). ¹H NMR (CDCL₃, 500 MHz): δ5.72 (d, 1H, J8.5 Hz), 5.35 (d, 1H, J3.5 Hz), 5.26 (dd, 1H, J3.5 Hz, 11.5 Hz), 4.67 (d, 1H, J8.5 Hz), 4.17 (dd, 1H, J6.5 Hz, 11.5 Hz), 4.12 (dd, 1H, 6.5 Hz, 11.5 Hz), 4.00 (dt, 1H, J8.5 Hz, 11.5 Hz), 3.92 (m, 2H), 3.53 (m, 1 H), 2.39 (m, 2H), 2.15 (s, 3H), 2.05 (s, 3H), 2.01 (s, 3H), 1.97 (s, 3H), 1.71 (m, 2H), 1.65 (m, 2H).

Synthesis of Trivalent GaINAc Targeting Moiety (NAG21)

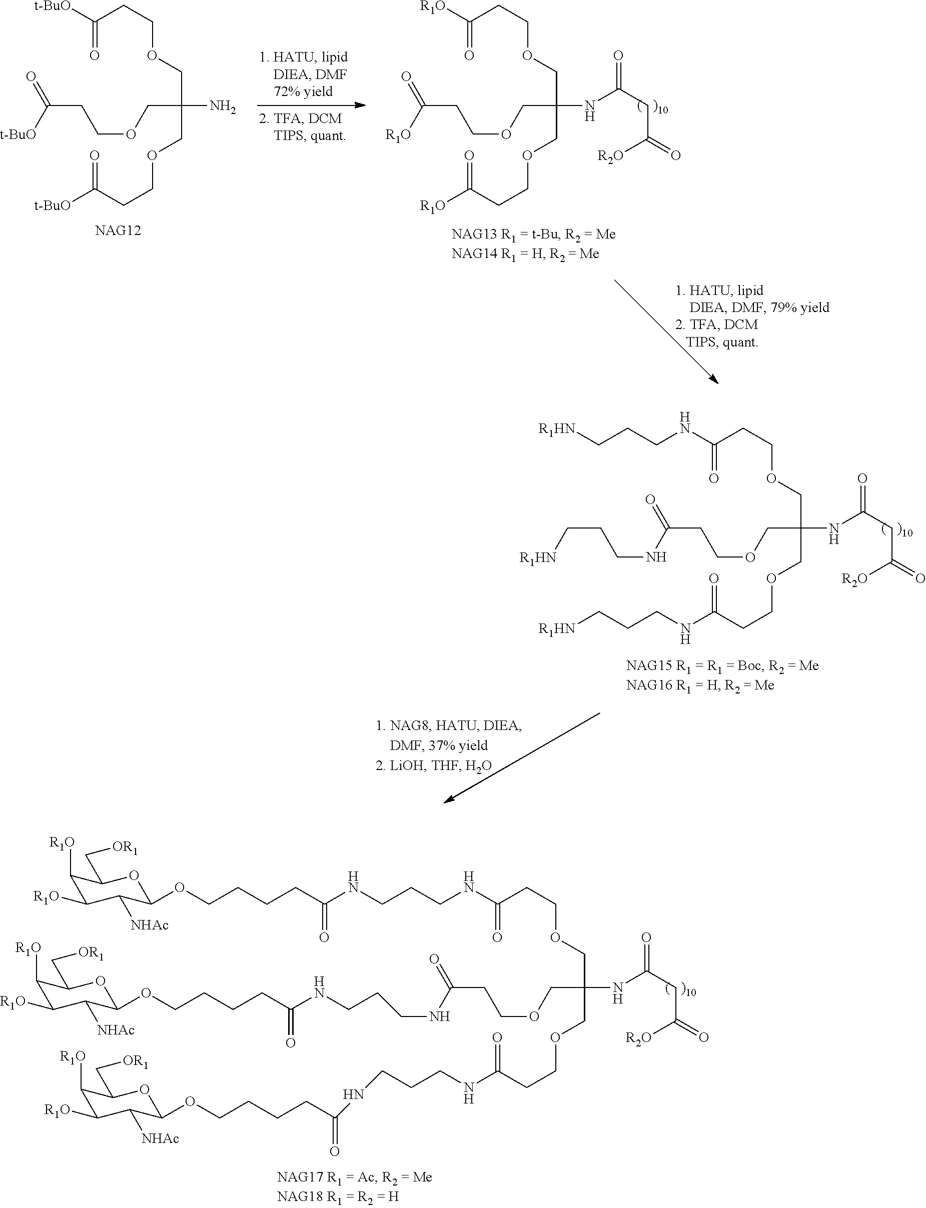

Preparation of tris-(carboxyethoxymethyl)-methylamido-dodecanedioate methyl ester (NAG14). To a solution of dodecanedioic acid methyl ester (211 mg, 0.42 mmol) activated with HATU (122 mg, 0.50 mmol) and DIEA (218 μL, 1.25 mmol) in DMF (2 mL) was added tris linker NAG12. After 1 h, the reaction mixture was concentrated in vacuo and purified by SiO₂ gel chromatography to afford NAG13 (214 mg, 70% yield). MALDI-TOF mass calcd C₃₈H₆₉NO₁₂: 731.48, Found: 755.10 [M+Na]. Tris t-butyl ester NAG13 was hydrolyzed with a TFA:TIPS:DCM (9:0.25:1) cocktail (10.25 mL) for 4 h and concentrated in vacuo to give tris acid NAG14. MALDI-TOF mass calcd C₂₆H₄₅NO₁₂: 563.29, Found: 565.33 [M+H].

Preparation of tris-(aminopropamido-ethoxymethyl)-methylamido-dodecanedioate methyl ester (NAG16). To a solution of tris acid NAG14 (230 mg, 0.41 mmol) activated with HATU (557 mg, 1.35 mmol) and DIEA (470 μL, 2.70 mmol) in DMF (4 mL) was added monoBoc 1,3-diaminopropane (250 mg, 1.44 mmol). After 1 h, the reaction was concentrated in vacuo and purified by SiO₂ gel chromatography to afford NAG15 (335 mg, 79% yield). MALDI-TOF mass calcd C₅₀H₉₃N₇O₁₅: 1031.67, Found: 1056.40 [M+Na]. Tris Boc linker NAG15 was treated with a TFA:TIPS:DCM (9:0.25:1) cocktail (10.25 mL) for 1 h and concentrated in vacuo to give tris amine NAG16. MALDI-TOF mass calcd C₃₅H₆₉N₇O₉: 731.51, Found: 733.18 [M+H].

Preparation of tris-GaINAc (NAG18): Monosaccharide NAG8 (192 mg, 0.43 mmol) was treated with HATU (163 mg, 0.43 mmol) and DIEA (150 μL, 0.86 mmol) in DMF (2 mL). After 30 min, a solution of NAG16 (80 mg, 0.11 mmol) in DMF (1 mL) was added and the mixture stirred for 1 h. The crude mixture was purified by SiO₂ gel chromatography to afford NAG17 (82 mg, 37% yield). Mass calcd C₉₂H₁₅₀N₁₀O₃₉: 2019.00, Found: 2041.85 [M+Na]. The peracetylated trimer GaINAc (82 mg, 0.04 mmol) was hydrolyzed upon treatment with LiOH.H₂O (34 mg, 0.81 mmol) in a THF:H₂O (3:1) solution (8 mL) to afford NAG18. MALDI-TOF mass calcd C₇₃H₁₃₀N₁₀O₃₀: 1626.89, Found: 1634.52 [M+Li].

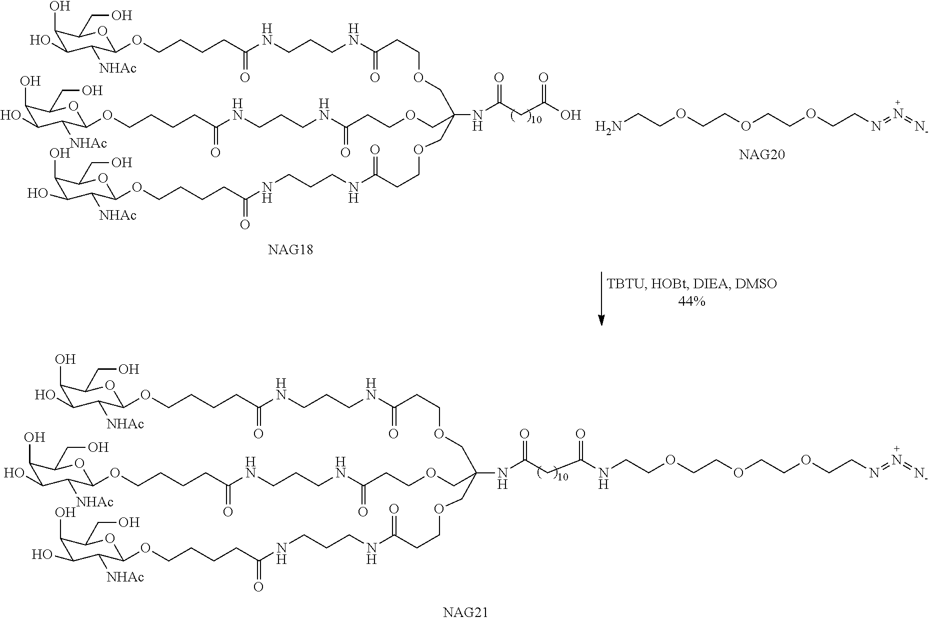

Preparation of azido-Peg₃-trimer GaINAc (NAG21). GaINAc trimer carboxylic acid NAG18 (60 mg, 0.03 mmol), azido-Peg₃-amine NAG20 (45.6 mg, 0.21 mmol), TBTU (23.8 mg, 0.07 mmol), HOBt (11.5 mg, 0.03 mmol), and DIEA (34 μL) were dissolved in DMSO (0.5 mL) and stirred 2 h. The base was removed in vacuo and the crude purified by RP-HPLC to afford NAG21 (24 mg, 44%). AP-ESI+Mass calcd C₈₁H₁₄₆N₁₄O₃₂: 1827.02, Found: 914.8 [M+2H]²⁺

Synthesis of Hexavalent Mannose Targeting Moiety (M9)

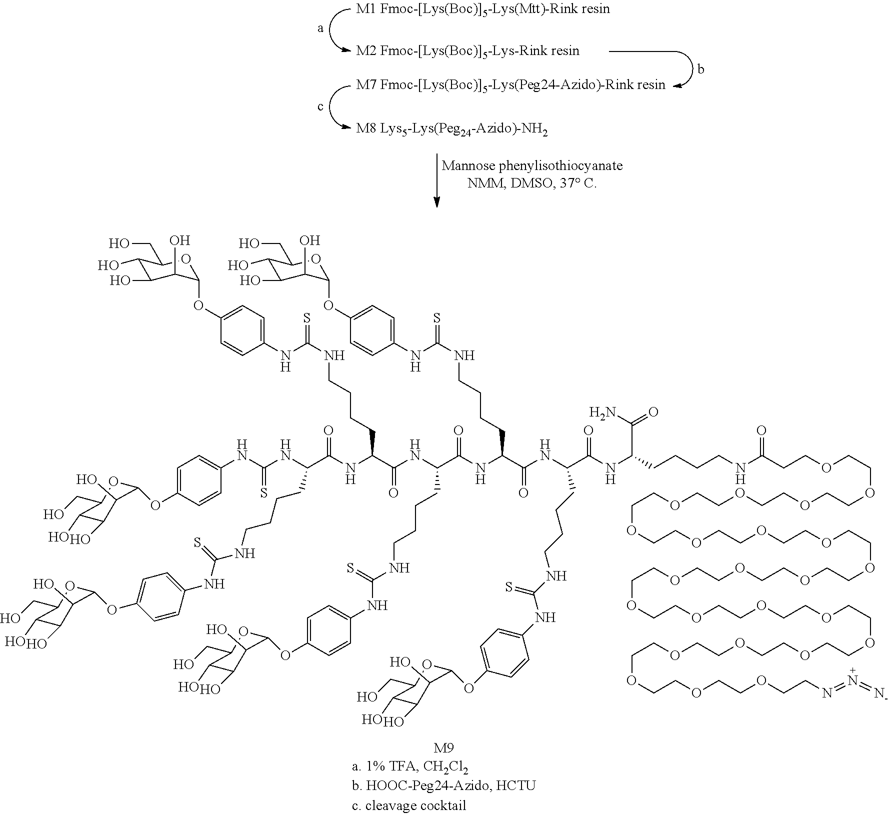

Preparation of Lys₆-Peg₂₄-Azide (M8). Peptide scaffold was synthesized using standard Fmoc chemistry on a Rink amide resin (0.61 mmol/g) with HCTU coupling and 20% piperidine deprotection. In short, peptide M1 was prepared on an automated synthesizer on a 100 μmol scale. After deprotection of Lys(Mtt), Azido-Peg₂₄ acid was coupled to provide M7. Release of the peptide from the resin using the cocktail TFA:TIPS:H₂O (92.5:2.5:5) afforded M8 (167.0 mg). MALDI TOF Mass calcd C₈₇H₁₇₄N₁₆O₃₁: 1940.4, Found: 1941.1

Preparation of Man₆-Lys₆-Peg₂₄-Azide (M9). Peptide scaffold M4 (167.0 mg) in DMSO (2 mL) was treated with mannose isothiocyanate and NMM (500 μL). The reaction was stirred at 37° C. and monitored by MALDI TOF until full conversion to the desired product was achieved (a total of 58 mgs of mannose isothyocyanate was added). The final product was purified by RP-HPLC to afford M9 (22 mg). MALDI-TOF mass calcd C₁₆₅H₂₆₄N₂₂O₆₇S_{6:} 3820.37, Found: 3843.79 [M+Na].

Synthesis of Trivalent Mannose Targeting Moiety (M15)

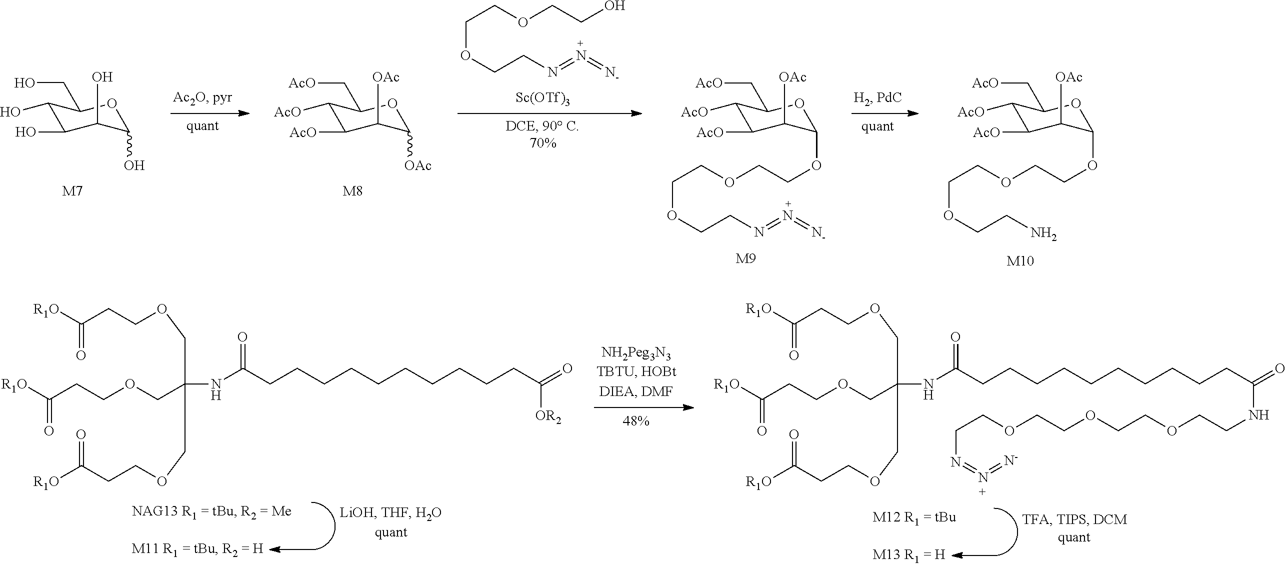

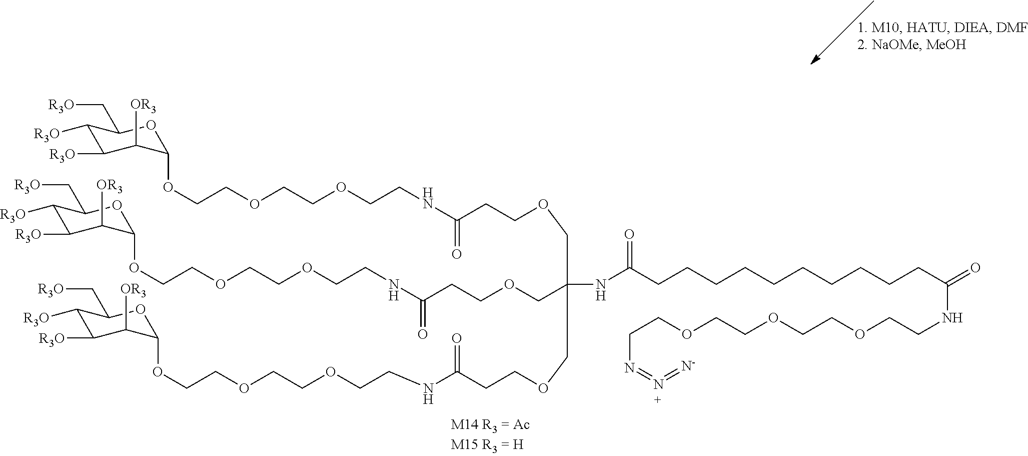

Preparation of azido tri-mannose (M15):D-Mannose was peracetylated by Ac₂O in pyridine overnight. Concentration by rotary evaporation followed by azeotroping with PhMe provided the penta-acetate (M8) in quantitative yield. Activation of M8 with Sc(OTf)₃ in the presence of commercially available azido-Peg₂ alcohol afforded azido-Peg₂ mannoside (M9), which was hydrogenated quantitatively to amine (M10). In the meanwhile, the methyl ester of tris linker (NAG13) was hydrolyzed to selectively to acid (M11). Coupling of commercially available azido Peg₃ amine to M11 by TBTU activation provided azido tris linker (M12). Treatment of tri t-butyl ester M12 with TFA gave tri-acid M13. Coupling of M10 to M13 was mediated by HATU and the crude mixture was globally de-acetylated to afford azido tri-mannose (M15).

Synthesis of Hexavalent Mannose Targeting Moiety (M30)

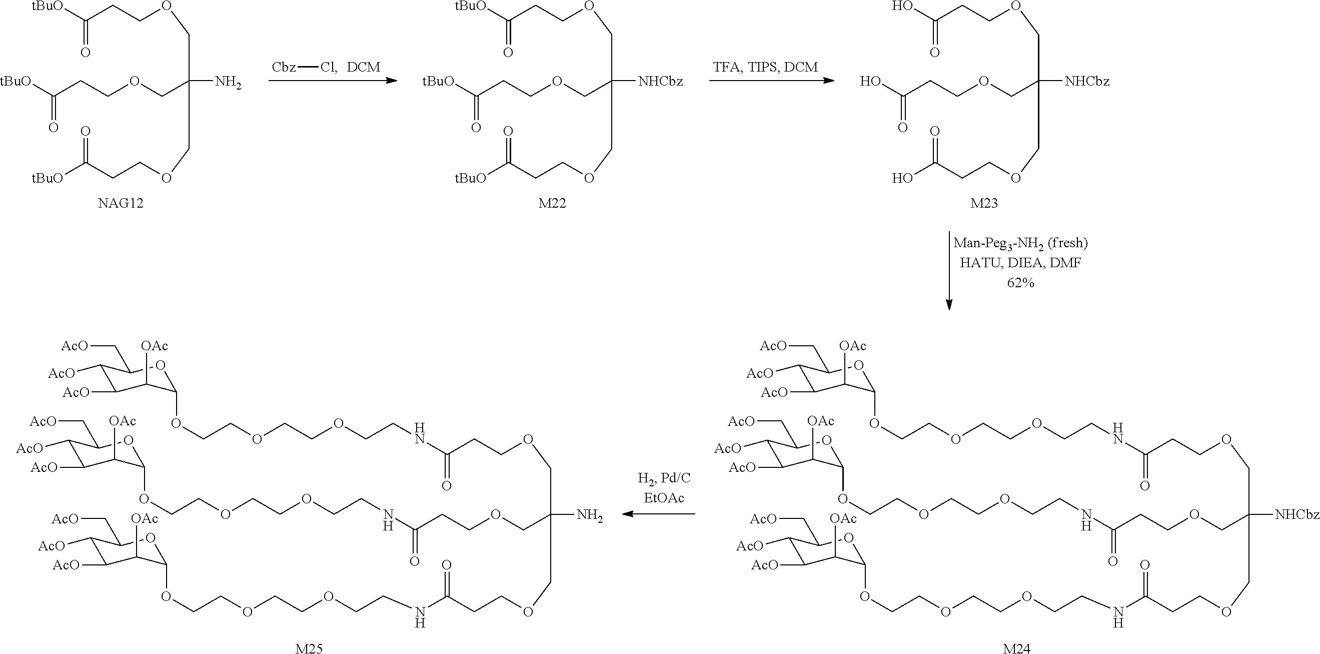

Preparation of N-carbobenzyloxy tris-(t-butoxycarboethoxymethyl)-methylamide (M22): To a solution of NAG12 (3.55 g, 7.02 mmol) in CH₂Cl₂ (12 mL) cooled in an ice bath was added Cbz-Cl (35% in PhMe, 7.3 mL) and TEA (3.9 mL). The reaction was warmed to rt and stirred overnight. The mixture was diluted with CH₂Cl₂ and washed with saturated NaHCO₃ (aq), dried over Na₂SO₄, concentrated in vacuo. The crude oil purified by SiO₂ chromatography to afford M22 (0.98 g, 22% yield). AP-ESI+Mass calcd C₃₃H₅₃NO₁₁: 639.4, Found: 662.4 [M+Na]⁺

Preparation of N-carbobenzyloxy tris-((2,3,4,6-tetra-O-acetyl-1-O-α-D-mannopyranosyl)-Peg₃-amidoethoxymethyl)-methylamide (M24): Tris-t-butyl ester M22 (0.97 g, 1.51 mmol) and TIPS (0.93 mL, 4.55 mmol) in CH₂Cl₂ (5 mL) was treated with TFA (20 mL) for 5 h. The mixture was concentrated in vacuo, the oily residue was washed with hexanes and dried under high vacuum to provide M23. AP-ESI+Mass calcd C₂₁H₂₉NO₁: 471.2, Found: 493.9 [M+Na]⁺

Crude M23 in DMF (5 mL) was cooled on an ice bath and treated with HATU (0.62 g, 1.63) and DIEA (0.65 mL, 3.71 mmol). After stirring for 20 min, a solution of M10 (0.89 g, 1.86 mmol) in DMF (5 mL) was added and the mixture was warmed to rt and stirred for 3 h. The solvent was removed in vacuo and the crude was dissolved in EtOAc and washed with saturated NaHCO₃ (aq), dried over Na₂SO₄, concentrated in vacuo. Purification by SiO₂ chromatography afforded M24 (0.49 g, 62% yield). MALDI-TOF Mass calcd C₈₁H₁₂₂N₄O₄₄: 1854.74, Found: 1850.14

Preparation of tris-((2,3,4,6-tetra-O-acetyl-1-O-α-D-mannopyranosyl)-Peg₃-amidoethoxymethyl)-methylamine (M25): A solution of M24 (0.49 g, 0.26 mmol) was dissolved in EtOAc (50 mL) with HOAc (0.2 mL) was degassed under vacuum and purged with Ar (g). Pd on activated carbon (0.16 g) was added and the mixture was evacuated and then purged with H₂ (g) thrice. Reaction was stirred for 2 days, catalyst removed by filtration, and mother liquor concentrated in vacuo to afford M25. AP-ESI+Mass calcd C₇₃H₁₁₆N₄O₄₂: 1720.7, Found: 1723.42

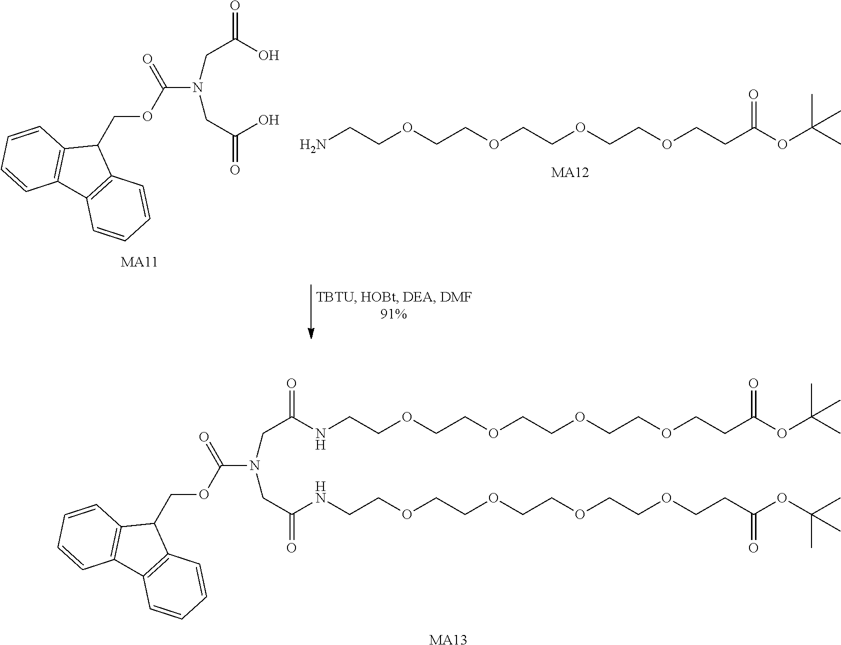

Preparation of N-Fmoc bis-imino-(acetamido-Peg₄ t-butyl ester) (MA13). N-Fmoc imino diacetic acid, MA11, (107 mg, 0.30 mmol) was treated with MA12 (212 mg, 0.66 mmol), TBTU (193 mg, 0.60 mmol), HOBt (92 mg, 0.60 mmol), and DIEA (209 μL, 1.20 mmol) in DMF for 2 h. The reaction was concentrated in vacuo and purified through SiO₂ gel chromatography to afford MA13 (250 mg, 91%). AP-ESI+Mass calcd C₄₉H₇₅N₃O₁₆: 961.51, Found: 962.6 [M+H]⁺, 984.6 [M+Na]⁺

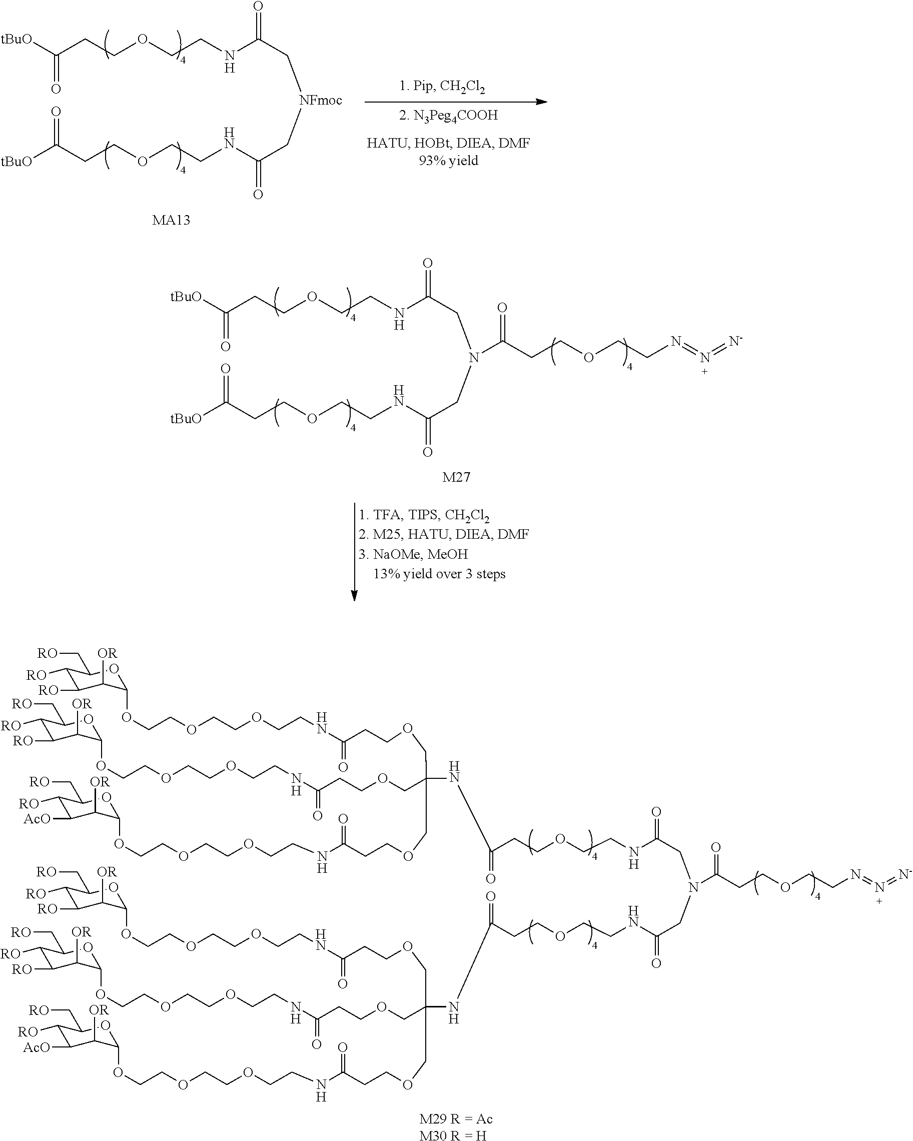

Preparation of azido-Peg₄-imido-bis-(acetamido-Peg₄-t-butyl ester) (M27): N-Fmoc MA13 (0.72 g, 0.75 mmol) in CH₂Cl₂ was treated with piperidine (0.75 mL) for 1 h. HPLCMS showed complete conversion to M26, AP-ESI+Mass calcd C₃₄H₆₅N₃O₁₄: 739.4, Found: 740.5 [M+H]⁺. The mixture was concentrated in vacuo and azeotroped with PhMe. Crude M26 was reacted with solution of azido Peg₄ acid (0.44 g, 1.51 mmol), HATU (0.57 g, 1.51 mmol), and DIEA (0.52 mL) in DMF (5 mL) for 1 h. After solvent removal in vacuo, the crude was dissolved in EtOAc, washed with sat NaHCO₃ (aq.), dried over Na₂SO₄, and concentrated in vacuo. Purification by SiO₂ chromatography afforded M27 (0.71 g, 93% yield, 2 steps). AP-ESI+Mass calcd C₄₅H₈₄N₆O₁₉: 1012.6, Found: 1013.6 [M+H]⁺

Preparation of azido-Peg₄-imido-bis-(trimer mannose) (M30): Imido linker M27 (0.69 g, 0.68 mmol) was treated with TIPS (0.28 mL, 1.36 mmol) and TFA (10 mL) to afford tri acid M28; AP-ESI+Mass calcd C₃₇H₆₈N₆O₁₉: 900.5, Found: 900.9 [M+H]⁺, 922.9 [M+Na]⁺. Volatiles were removed in vacuo and M28 dried under high vacuum. Di-acid M28 (82.0 mg, 0.09 mmol) was activated with HATU (75 mg, 0.2 mmol) and DIEA (0.28 mL) in DMF (2 mL) at 0° C. After 30 min, a solution of M25 (0.26 mmol) in DMF (2 mL) was added and the mixture was warmed to rt and stirred for 2h. RP-HPLCMS showed complete conversion to M29; Mass calcd C₁₈₃H₂₉₆N₁₄O₁₀₁: 4305.84. MALDI-TOF Found: 4303.36 AP-ESI+Found: 1436.1 [M+3H]³⁺, 1077.3 [M+4H]⁴⁺. The reaction was diluted with CH₂CL₂ washed with sat NaHCO₃ (aq.), dried over Na₂SO₄, and concentrated in vacuo. The crude M29 oil (538 mg) dissolved in MeOH (20 mL) was treated with NaOMe (25 wt % in MeOH, 0.5 mL) for 1 h. RP-HPLCMS showed complete conversion to M30. The reaction was quenched by addition of Dowex H+ resin to neutralize. The crude material was purified by HPLC to afford M30 (38.1 mg, 13% yield over 3 steps). Mass calcd C₁₃₅H₂₄₈N₁₄O₇₇: 3297.59, MALDI-TOF Found: 3318.61 [M+Na]⁺ AP-ESI+Found: 1100.0 [M+3H]³⁺, 825.3 [M+4H]⁴⁺.

### Conjugation of Delivery Domains

Copper-THPTA complex preparation:

A 5 mM aqueous solution of copper sulfate pentahydrate (CuSO₄-5H₂O) and a 10 mM aqueous solution of Tris(3-hydroxypropyltriazolylmethyl)amine (THPTA) were mixed 1:1 (v/v) (1:2 molar ratio) and allowed to stand at room temperature for 1 hour. This complex can be used to catalyze Hüisgen cycloaddition, e.g., in the reaction shown in the Conjugation Scheme below.

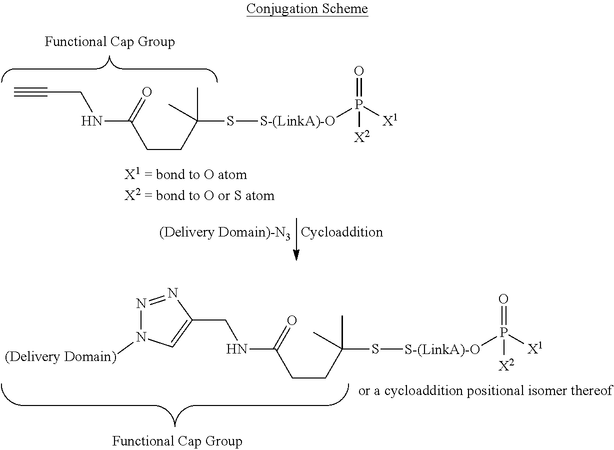

As shown in the Conjugation Scheme above, a Delivery Domain can be attached to the mononucleotide of the invention using, e.g., a cycloaddition reaction (e.g., Hüisgen cycloaddition). Hüisgen cycloaddition may be carried out with the copper-THPTA catalyst (see above).

The following conjugates can be prepared using the methods described herein:

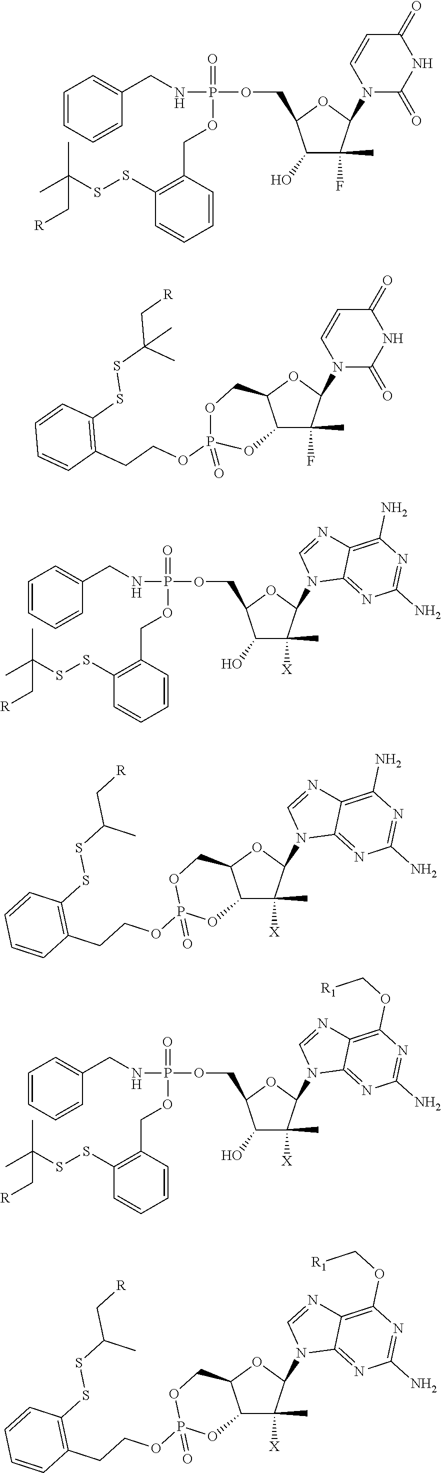

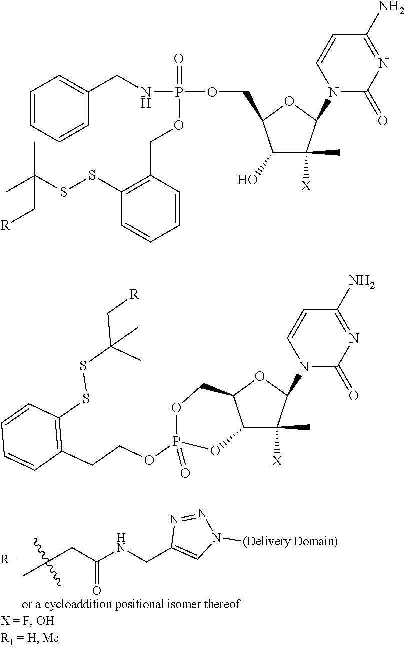

Delivery Domain can be, e.g., a targeting moiety (e.g., GaINAc, Mannose, Lipid, etc.), a cell penetrating peptide, or an endosomal escape moiety.

### Example 2

### HCV Replication Assays

The antiviral activity of the test compounds was assessed in the wild type GT1b (Con1), GT1a (H77), and GT1b/2a, GT1b/3a, GT1b/4a and GT1b/5a NS5B chimeric replicons, as well as the NS5B mutant replicons listed in Table 2a and 2b.

To generate HCV NS5B chimeric replicons, the GT1 b replicon was used as a backbone with the NS5B gene replaced with the NS5B gene of GT2a, GT3a, GT4a and GT5a derived from clinical isolates. These NS5B genes were cloned into the GT1b backbone and were confirmed by sequencing.

The HCV replicon mutants were generated by site-directed mutagenesis (SDM). The SDM was performed by PCR and the PCR fragments were inserted into the backbone replicon construct. The inserted PCR fragments and the mutants were confirmed by sequencing.

All the replicon assays were luciferase based in Huh-7 cells, either in stable format (GT1b and GT1a) or by transient transfection by electroporation (chimeric and mutant replicons). For a standard HCV replicon assay, stable or transiently transfected Huh-7 cells were seeded in 96-well plates (5,000 cells/well), cultured in DMEM containing 10% FBS, and incubated at 37° C., 5% CO₂. On the following day, test compounds were diluted with assay media and added to the appropriate wells (final DMSO concentration in the cell culture medium was 0.5%). Assay reference positive control was included in each run to ensure assay performance. Cells were incubated at 37° C., 5% CO₂ for 72 hours, at which time the cells were still sub-confluent. The antiviral activity was determined by measuring replicon reporter firefly luciferase activity using Bright Glo kit in accordance with the protocol provided by the supplier (Promega). The toxicity of the test compounds was assessed by CytoTox-1 cell proliferation assay (Promega). The half maximal effective concentration (EC₅₀) and the half maximal toxic concentration (TC₅₀) values were calculated using the GraphPad Prism software.

TABLE 2
Genotype Profiling of Nucleotide Compounds

| | | | | | | | Selectivity |
|---|---|---|---|---|---|---|---|
| Com- | GT1a | GT1b | GT2a | GT3a | GT4a | GT5a | Index |
| pound | EC50 (nM) | | | | | | (TC₅₀/EC₅₀) |
| 4A/4B | — | 40 | — | — | — | — | 226 |
| 6A | — | 840 | — | — | — | — | >12 |
| 6B | — | 730 | — | — | — | — | >14 |
| 7A | — | 9 | — | 8 | — | — | >500 |
| 7B | — | 20 | — | 14 | — | — | >500 |
| 8A | — | 10 | — | 6 | — | — | >500 |
| 8B | — | — | — | — | — | — | — |
| 9A | — | 7 | — | 6 | — | — | >500 |
| 9B | — | 13 | — | 29 | — | — | >500 |
| 10A | — | 40 | — | 41 | — | — | >500 |
| 10B | — | 21 | — | 21 | — | — | >500 |
| 16A | — | 20 | — | 6 | — | — | >500 |
| 16B | — | 40 | — | 5 | — | — | >250 |
| 18A | — | 50 | — | — | — | — | >200 |
| 18B | — | — | — | — | — | — | — |
| 19A | 169 | 177 | 151 | 228 | 77 | 288 | — |
| 19B | 858 | 1034 | 780 | 1342 | 344 | 1350 | — |
| sofosbuvir | 40-61 | 36-66 | 36 | 90-95 | 54 | 65 | >100 |

TABLE 3
Genotype and Mutant Profiling of Select Nucleotide Compounds

| | | sofosbuvir | 7A | 9A |
|---|---|---|---|---|
| Genotype | Mutant | EC50 (nM) | | |
| GT1a | — | 40-61 | 5-8 | 3-6 |
| | S282T | 194-694 | 60 | 6-35 |
| | L159F* | 80 | 11 | 10 |
| GT1b | — | 36-66 | 5-8 | 4-5 |
| | S282T | 446-554 | 65 | 23-25 |
| | S96T | 62-314 | 8 | 7 |
| | L159F* | 63 | 15-17 | 9 |
| | C316N* | 25 | 9 | 4 |
| GT2a | — | 36 | 3 | 3 |
| GT3a | — | 90-95 | 8 | 4-6 |
| | S282T | 84-572 | 44 | 6-18 |
| | L159F* | 134 | 15 | 8 |
| | V321A* | 166 | 12 | 12 |
| GT4a | — | 54 | 5 | 3 |
| GT5a | — | 65 | 8 | 2 |

*clinically identified mutants

### Nucleoside Phosphoester Stability in Serum:

Mononucleotide stock solutions were prepared at 10 mM in DMSO; 10 μL of each stock solution was added to 1 mL of serum (mouse, rat, and human) to provide 100 μM of final compound concentration. Samples were incubated at 37° C.; 100 μL aliquots were removed at selected time points and added directly into 200 μL cold acetonitrile to precipitate protein. Samples were centrifuged at 14K RPM for 30 min at 4° C.; 100 μL of the resulting supernatant was combined with 100 μL of water+0.1% formic acid and subjected to LCMS analysis as described below.

LCMS conditions were as follows:

Column: Phenomenex Kinetex 5u C18, 2.1×50 mm;

Mobile phase A: water+0.1% formic acid:

Mobile phase B: acetonitrile+0.1% formic acid;

Flow rate: 0.4 mL/min;

Injection volume: 10μL;

Gradient: 5-95%;

B in 2.5 min;

Detection: ESI positive and negative m/z 250-800.

Extracted ion chromatograms were generated using M+1 H or M-1 H ions of the intact theoretical MW of each nucleotide prodrug and integrated peak areas measured using LCMS processing software. Quantification was performed by comparison to an external standard curve of compounds spiked into appropriate serum and processed as above. Data plots represented as ratios of compound remaining compared to t=0 time point.

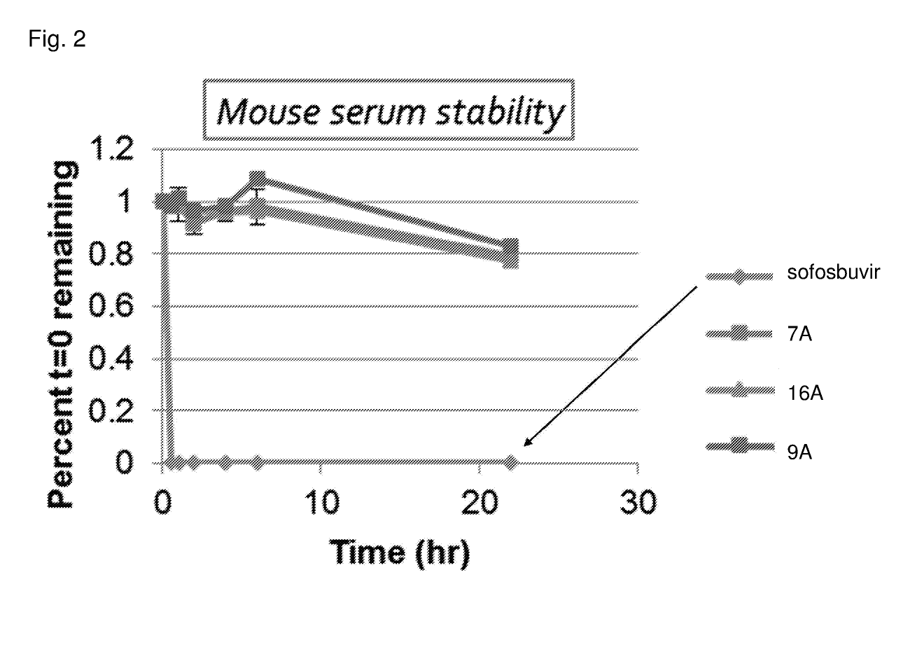

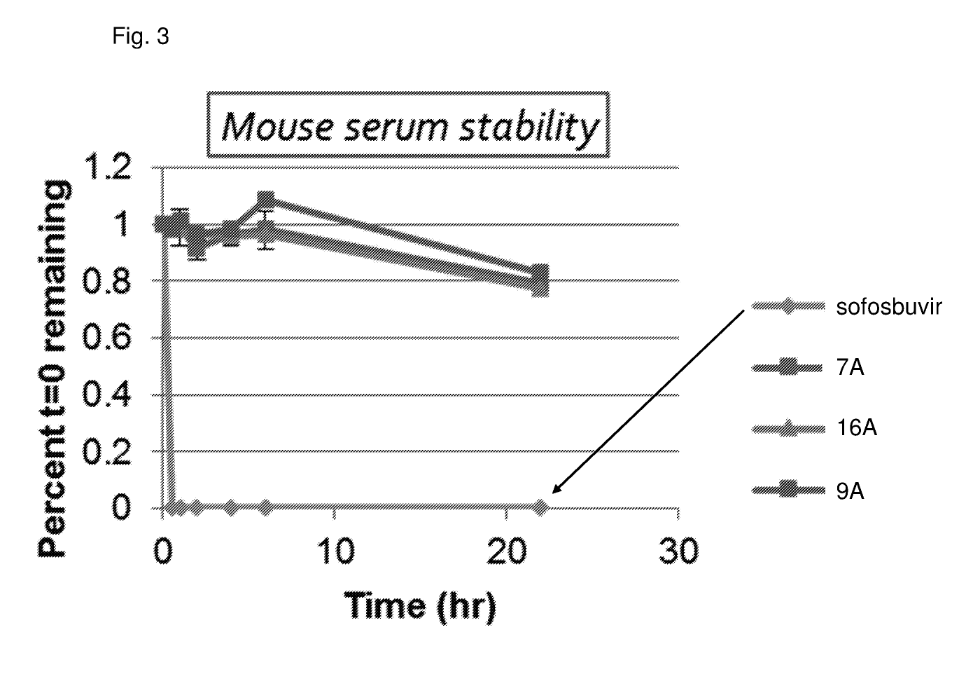

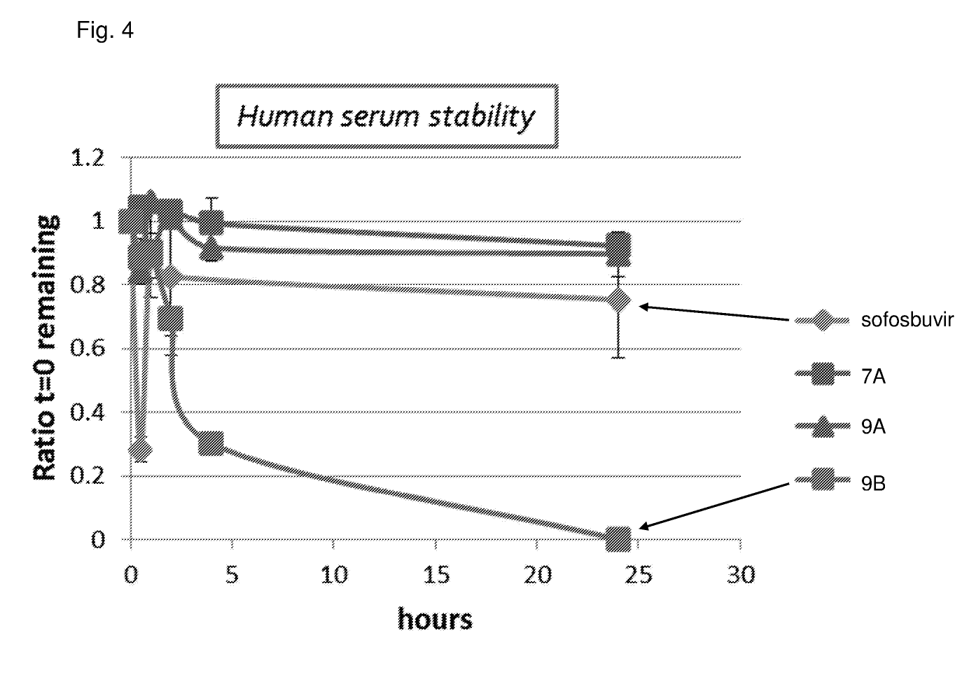

The results of this test are shown in FIGS. 2, 3, and 4.

In another test, mononucleotide stock solutions were prepared at 10 mM in DMSO, and 10 μL of each stock solution was added to 1 mL of fetal bovine serum (FBS) to provide 100 μM of final compound concentration. These samples incubated for 24 h in a 37° C. water bath and 100 μL aliquots removed at t=0, 1, 2, 4, 6 and 24 hours. Individual samples were precipitated with 200 μL cold acetonitrile, the debris was pelleted at 14K RPM for 30 min at 4° C., and the supernatant was removed and subjected to LCMS analysis as described below.

LCMS Method:

Column: Kinetex 5u C8 100A, 2.1×50 mm

Mobile phase A: 95:5 H₂O:acetonitrile, 10 mM ammonium acetate, 0.01% formic acid

Mobile phase B: 95:5 acetonitrile:H₂O, 10 mM ammonium acetate, 0.01% formic acid

Flow rate: 0.4 mL/min

Injection volume: 7.5 μL

Gradient: 0-100% B in 2.5 min

Detection: A254, m/z 100-1000 (coneV=30)

Extracted ion chromatograms generated using M+1H and ammonium adduct for each compound.

The results of this test are provided in Tables 4, 5, and 6

TABLE 4

| Time (hours) | AUC | % t = 0 |
|---|---|---|
| 0 | 8933 | 100.0 |
| 1 | 8595 | 96.2 |
| 2 | 8624 | 96.5 |
| 4 | 7952 | 89.0 |
| 6 | 8180 | 91.6 |
| 24 | 9129 | 102.2 |

Table 4 shows the fetal bovine serum stability data for sofosbuvir; ESI⁺, EIC: m/z=530 [M+H]⁺and 547 [M+NH₄⁺].

TABLE 5

| Time (hours) | AUC | % t = 0 |
|---|---|---|
| 0 | 6052 | 100.0 |
| 1 | 5813 | 96.1 |
| 2 | 5973 | 98.7 |
| 4 | 6018 | 99.4 |
| 6 | 5783 | 95.6 |
| 24 | 5818 | 96.1 |

Table 5 shows the fetal bovine serum stability data for compound 4; ESI⁺, EIC: m/z=654 [M+H]⁺ and 671 [M+NH₄⁺].

TABLE 6

| Time (hrs) | AUC | % t = 0 |
|---|---|---|
| 0 | 5006 | 100.0 |
| 1 | 5056 | 101.0 |
| 2 | 5055 | 101.0 |
| 4 | 5052 | 100.9 |
| 6 | 4633 | 92.5 |
| 24 | 4401 | 87.9 |

Table 6 shows the fetal bovine serum stability data for compound 7A; ESI⁺, EIC: m/z=547 [M+H]⁺ and 564 [M+NH₄⁺].

### Example 3

### Nucleoside Triphosphate Measurement In Vitro and In Vivo

For in vitro experiments, approximately 5,000,000 isolated hepatocyte cells were plated onto collagen coated dishes and allowed to adhere for 6 hours. Dosing solution containing nucleotide prodrugs in growth media were exposed to the cells for up to 24 hours. Cells were harvested by scraping from the dish, pelleted, and kept on ice. For in vivo experiments, individual mice or rats were exposed to nucleotide prodrugs either by intravenous tail vein injection in physiological saline solution or by oral gavage (PO dosing) in a PEG-methylcellulose mixture. At selected time points, animals were euthanized by CO₂ overdose, livers were dissected, and 200 mg sections of livers were snap frozen in liquid nitrogen.

Hepatocyte cells or liver tissue from above were suspended in cold 60% methanol, 10 mM EDTA, and 50mM ammonium acetate and homogenized using bead disruption. Debris was pelleted, and supernatant was analyzed directly by anion exchange LCMS as described below.

LCMS conditions were as follows: column-Thermo BioBasic AEX, 5 μm, 2.1×100 mm; mobile phase A-30:70 acetonitrile:50 mM ammonium acetate pH=6, mobile phase B-30:70 acetonitrile:10 mM ammonium acetate pH=10; flow rate-0.4 mL/min; injection volume: 25-100 μL; gradient: 30-95% B in 2 min, hold at 95% B for additional 3 min; detection: ESI negative m/z 250-800. Extracted ion chromatograms were generated using M-1 H ions of the intact theoretical MW of the triphosphate compound, and integrated peak areas were measured using LCMS processing software. Quantification performed by comparison to an external standard curve of appropriate triphosphate compounds spiked into blank matrix.

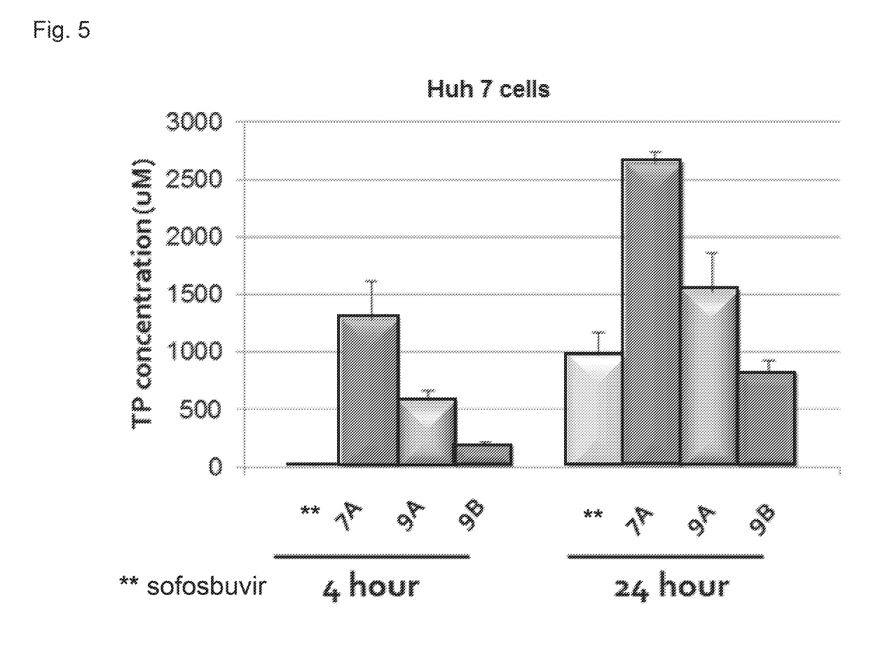

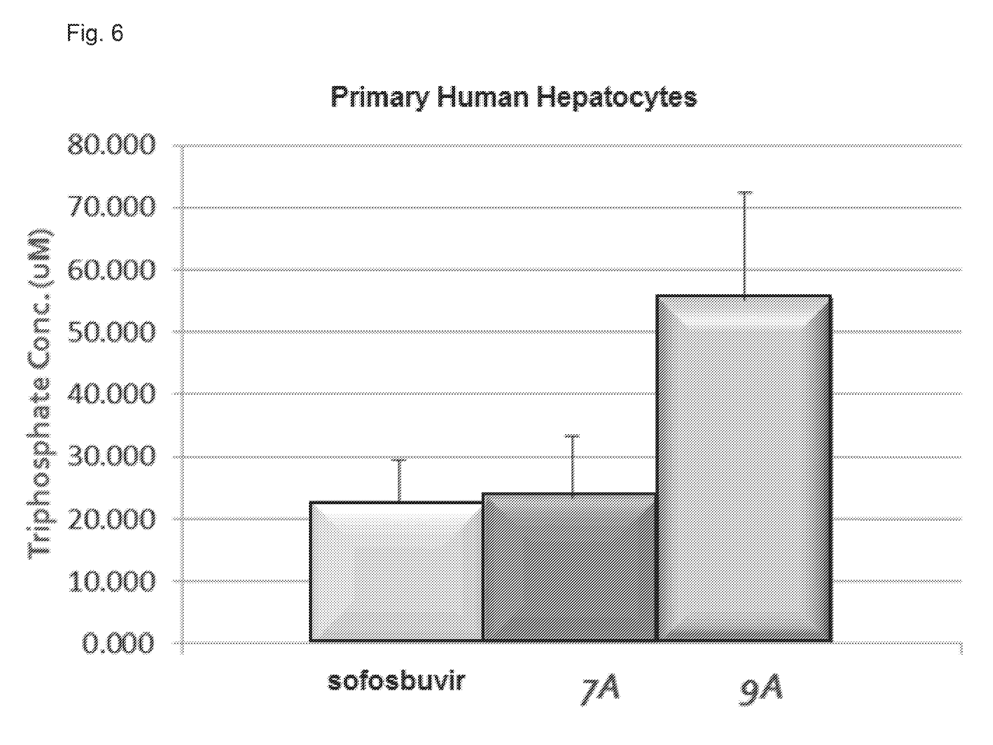

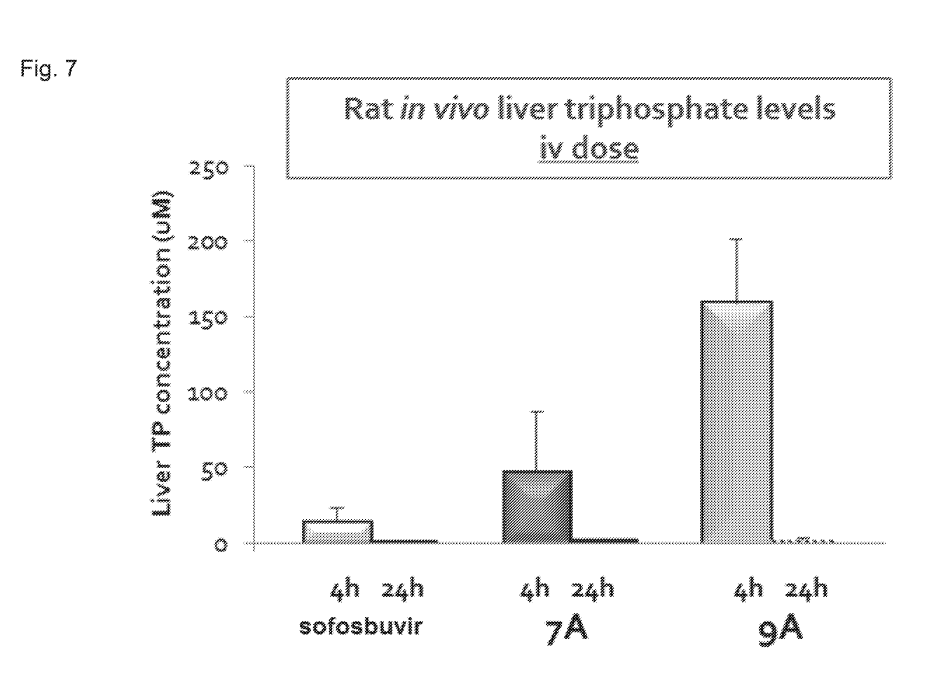

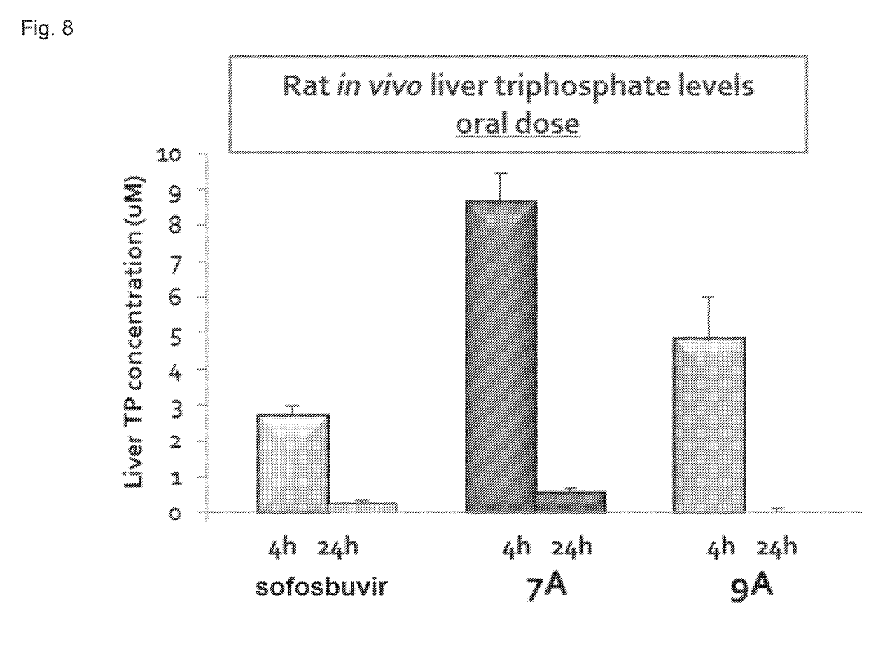

The results are provided in FIGS. 5, 6, 7, and 8.

### Example 4

### Nucleoside Phosphoester Stability in Simulated Gastric Fluid

Test compounds at 2 μM were incubated at 37° C. with simulated gastric fluid (SGF, 0.2% (w/v) sodium chloride in 0.7% (v/v) hydrochloric acid, deionized water, 0.3% pepsin (w/v), pH 1.2). Duplicate samples were used. Samples were removed at 0, 15, 30, 60, 120, 360 and 1440 min, immediately mixed with cold acetonitrile containing internal standard (IS), and stored at −80° C. before analysis. Omeprazole was used as a positive control. Samples were analyzed by LC/MS/MS method, and disappearance of test compound was assessed by comparison of peak area ratios of analyte/IS and reported as % test compound remaining at each time point.

The results are provided in Table 7.

TABLE 7
Stability of Nucleotide Phosphoesters in Simulated Gastric Fluid

| | % Remaining | | | | |
|---|---|---|---|---|---|
| Incubation | | | | | Control |
| Time (min) | sofosbuvir | 7A | 9A | 9B | (Omeprazole) |
| 0 | 100.00 | 100.00 | 100.00 | 100.00 | 100.00 |
| 15 | 93.47 | 95.13 | 107.89 | 95.72 | 79.48 |
| 30 | 99.50 | 99.01 | 102.51 | 98.51 | 66.64 |
| 60 | 99.63 | 92.49 | 97.63 | 100.39 | 34.79 |
| 120 | 95.33 | 91.52 | 94.89 | 92.48 | 33.00 |
| 360 | 87.35 | 91.60 | 91.06 | 65.99 | 6.17 |
| 1440 | 61.36 | 70.36 | 76.22 | 21.04 | 1.24 |

### Example 5

### Nucleoside Phosphoester Stability in Simulated Intestinal Fluid

Test compounds at 2 μM were incubated at 37° C. with simulated intestinal fluid (SIF), which contains 0.68% (w/v) monobasic potassium phosphate and 1% (w/v) pancreatin in ultra-pure water (pH 6.8). Duplicate samples were used. Samples were removed at 0, 15, 30, 60, 120, 360 and 1440 min, immediately mixed with cold acetonitrile containing an internal standard (IS), and stored at −80° C. before analysis. Chlorambucil was used as a positive control. Samples were analyzed by a LC/MS/MS method, and disappearance of test compound was assessed by comparison of peak area ratios of analyte/IS and reported as % test compound remaining at each time point.

The results are provided in Table 8.

TABLE 8
Stability of Nucleoside Phosphoesters in Simulated Intestinal Fluid

| | % Remaining | | | | |
|---|---|---|---|---|---|
| Incubation | | | | | Control |
| Time (min) | sofosbuvir | 7A | 9A | 9B | (Chlorambucil) |
| 0 | 100.00 | 100.00 | 100.00 | 100.00 | 100.00 |
| 15 | 5.57 | 102.65 | 99.21 | 98.51 | 81.31 |
| 30 | 0.00 | 104.34 | 103.81 | 91.63 | 64.39 |
| 60 | 0.00 | 111.04 | 103.14 | 99.30 | 34.82 |
| 120 | 0.00 | 101.89 | 109.48 | 97.42 | 5.08 |
| 360 | 0.00 | 121.95 | 101.64 | 72.51 | 0.00 |
| 1440 | 0.00 | 51.70 | 98.27 | 33.69 | 0.00 |

### Other Embodiments

Various modifications and variations of the described compositions and methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other Embodiments are in the Claims

1. A mononucleotide comprising a nucleobase bonded to a sugar having a 3′-carbon and a 5′-carbon, wherein said 5′-carbon is bonded to a phosphorus (V) atom of a phosphate group through an oxygen atom, said phosphorus (V) atom being bonded to
(i) one and only one disulfide bioreversible group through an oxygen atom; and
(ii) (a) optionally substituted amino, optionally substituted C₁₋₆ alkoxy, optionally substituted C₆₋₁₄ aryloxy, or optionally substituted C₁₋₉ heteroaryloxy; or (b) said 3′-carbon through an oxygen atom.

2. The mononucleotide of claim 1, wherein said phosphate group comprises one and only one phosphorus (V) atom.

3. The mononucleotide of claim 1, wherein said phosphorus (V) atom is bonded to said 3′-carbon through said oxygen atom.

4. The mononucleotide of claim 1, wherein said phosphorus (V) atom is bonded to optionally substituted amino, optionally substituted C₁₋₆ alkoxy, optionally substituted C₆₋₁₄ aryloxy, or optionally substituted C₁₋₉ heteroaryloxy.

5. The mononucleotide of claim 4, wherein said phosphorus (V) atom is bonded to optionally substituted amino or optionally substituted C₆₋₁₄ aryloxy.

6. The mononucleotide of claim 5, wherein said phosphorus (V) atom is bonded to an optionally substituted amino.

7. The mononucleotide of claim 1, wherein said disulfide bioreversible group has a structure of formula (I):
(i) G-S—S-(LinkA)-X (I),
wherein
G is a functional cap group,
LinkA is a linker having a molecular weight greater than or equal to 28 Da, and
X is a bond to the oxygen atom of said phosphate group.

8. The mononucleotide of claim 1 having a structure of formula (II): 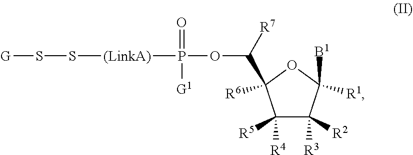
or a pharmaceutically acceptable salt or a phosphorus diastereomer thereof,
wherein
G is a functional cap group;
LinkA is a linker;
B¹ is a nucleobase;
R¹ is H, azido, cyano, optionally substituted C₁₋₆ alkyl, optionally substituted C₂₋₆ alkenyl, or optionally substituted C₂₋₆ alkynyl;
each of R² and R³ is independently H, amino, azido, optionally substituted C₁₋₆ alkyl, optionally substituted C₁₋₆ heteroalkyl, optionally substituted C₂₋₆ alkenyl, optionally substituted C₂₋₆ alkynyl, halo, cyano, hydroxy, or optionally substituted C₁₋₆ alkoxy;
G¹ is optionally substituted amino, optionally substituted C₁₋₆ alkoxy, optionally substituted C₆₋₁₄ aryloxy, or optionally substituted C₁₋₉ heteroaryloxy, and R⁴ is hydroxy, optionally substituted C₁₋₆ alkoxy, optionally substituted amino, or azido, or G¹ and R⁴ combine to form —O—;
R⁵ is H, optionally substituted C₁₋₆ alkyl, optionally substituted C₁₋₆ heteroalkyl, optionally substituted C₂₋₆ alkenyl, optionally substituted C₂₋₆ alkynyl, or cyano;
R⁶ is H, azido, cyano, halo, optionally substituted C₁₋₆ alkyl, optionally substituted C₂₋₆ alkenyl, or optionally substituted C₂₋₆ alkynyl; and
R⁷ is H or optionally substituted C₁₋₆ alkyl.

9. The mononucleotide of claim 8, wherein G is a blocking group, a delivery domain, or a dye.

10. A mononucleotide of formula (II): 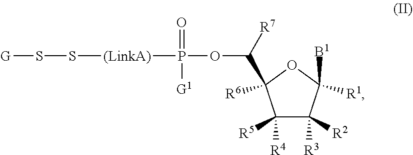
or a pharmaceutically acceptable salt or a phosphorus diastereomer thereof,
wherein
G is optionally substituted C₃₋₁₀ alkyl, optionally substituted C₃₋₁₀ heteroalkyl, optionally substituted C₆₋₁₄ aryl, optionally substituted C₁₋₉ heterocyclyl;
LinkA consists of 1, 2, or 3 monomers independently selected from the group consisting of optionally substituted C₁₋₆ alkylene, optionally substituted C₁₋₆ heteroalkylene, optionally substituted C₆₋₁₄ arylene, optionally substituted C₁₋₉ heterocyclylene, optionally substituted aza, O, and S; wherein LinkA does not comprise two contiguous atoms selected from the group consisting of O and S, and wherein the monomer attached to the oxygen atom of said phosphate group is optionally substituted C₁₋₆ alkylene;
B¹ is a nucleobase;
R¹ is independently H, azido, cyano, optionally substituted C₁₋₆ alkyl, optionally substituted C₂₋₆ alkenyl, or optionally substituted C₂₋₆ alkynyl;
each of R² and R³ is independently H, amino, azido, optionally substituted C₁₋₆ alkyl, optionally substituted C₁₋₆ heteroalkyl, optionally substituted C₂₋₆ alkenyl, optionally substituted C₂₋₆ alkynyl, halo, cyano, hydroxy, or optionally substituted C₁₋₆ alkoxy;
G¹ is optionally substituted amino, optionally substituted alkoxy, optionally substituted C₆₋₁₄ aryloxy, or optionally substituted C₁₋₉ heteroaryloxy, and R⁴ is hydroxy, optionally substituted C₁₋₆ alkoxy, optionally substituted amino, or azido, or G¹ and R⁴ combine to form —O—; and
R⁵ is H, optionally substituted C₁₋₆ alkyl, optionally substituted C₁₋₆ heteroalkyl, optionally substituted C₂₋₆ alkenyl, optionally substituted C₂₋₆ alkynyl, or cyano;
R⁶ is H, azido, cyano, halo, optionally substituted C₁₋₆ alkyl, optionally substituted C₂₋₆ alkenyl, or optionally substituted C₂₋₆ alkynyl; and
R⁷ is H or optionally substituted C₁₋₆ alkyl.

11. The mononucleotide of claim 8, wherein R¹ is H.

12. The mononucleotide of claim 8, wherein R² is optionally substituted C₁₋₆ alkyl.

13. The mononucleotide of claim 8, wherein R³ is hydroxy, optionally substituted C₁₋₆ alkoxy, or halo.

14. The mononucleotide of claim 13, wherein R³ is halo.

15. The mononucleotide of claim 8, wherein R⁵ is H.

16. The mononucleotide of claim 8, wherein R⁶ is H.

17. The mononucleotide of claim 8, wherein R⁷ is H or Me.

18. The mononucleotide of claim 8, wherein G¹ is optionally substituted amino or optionally substituted C₆₋₁₄ aryloxy.

19. The mononucleotide of claim 18, wherein G¹ is optionally substituted amino.

20. The mononucleotide of claim 8, wherein R⁴ is hydroxy.

21. The mononucleotide of claim 8, wherein G¹ and R⁴ combine to form —O—.

22. The mononucleotide of claim 7, wherein G is a delivery domain.

23. The mononucleotide of claim 22, wherein said delivery domain comprises a targeting moiety, an endosomal escape moiety, or a cell penetrating peptide.

24. The mononucleotide of claim 23, wherein said delivery domain comprises a targeting moiety.

25. The mononucleotide of claim 24, wherein said targeting moiety comprises from 1 to 10 carbohydrates.

26. The mononucleotide of claim 25, wherein each said carbohydrate is independently GaINAc or mannose.

27. The mononucleotide of claim 26, wherein said carbohydrate is GaINAc.

28. The mononucleotide of claim 27, wherein said carbohydrate is mannose.

29. The mononucleotide of claim 24, wherein said targeting moiety is a lipid.

30. The mononucleotide of claim 7, wherein G is a blocking group.

31. The mononucleotide of claim 30, wherein G is an optionally substituted C₃₋₁₀ alkyl, optionally substituted C₃₋₁₀ heteroalkyl, optionally substituted C₆₋₁₄ aryl, or optionally substituted C₁₋₉ heterocyclyl.

32. The mononucleotide of claim 7, wherein LinkA consists of 1, 2, or 3 monomers independently selected from the group consisting of optionally substituted C₁₋₆ alkylene, optionally substituted C₁₋₆ heteroalkylene, optionally substituted C₆₋₁₄ arylene, optionally substituted C₁₋₉ heterocyclylene, optionally substituted aza, O, and S; wherein LinkA does not comprise two contiguous atoms selected from the group consisting of O and S, and wherein the monomer attached to the oxygen atom of said phosphate group is optionally substituted C₁₋₆ alkylene.

33. The mononucleotide of claim 32, wherein LinkA consists of 1, 2, or 3 monomers independently selected from the group consisting of optionally substituted C₁₋₆ alkylene, optionally substituted C₆₋₁₄ arylene, and O.

34. The mononucleotide of claim 33, wherein LinkA consists of 1 or 2 monomers independently selected from the group consisting of optionally substituted C₁₋₆ alkylene and optionally substituted C₆₋₁₄ arylene.

35. A mononucleotide: 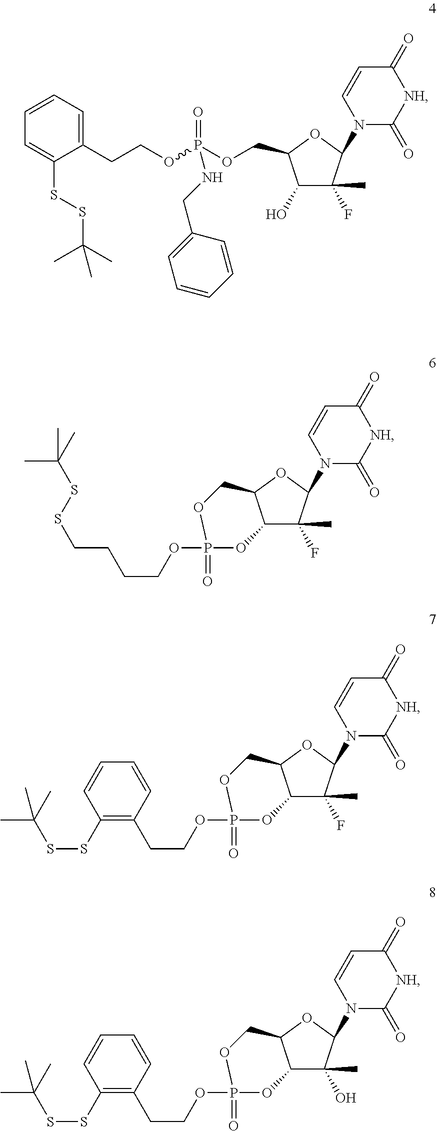 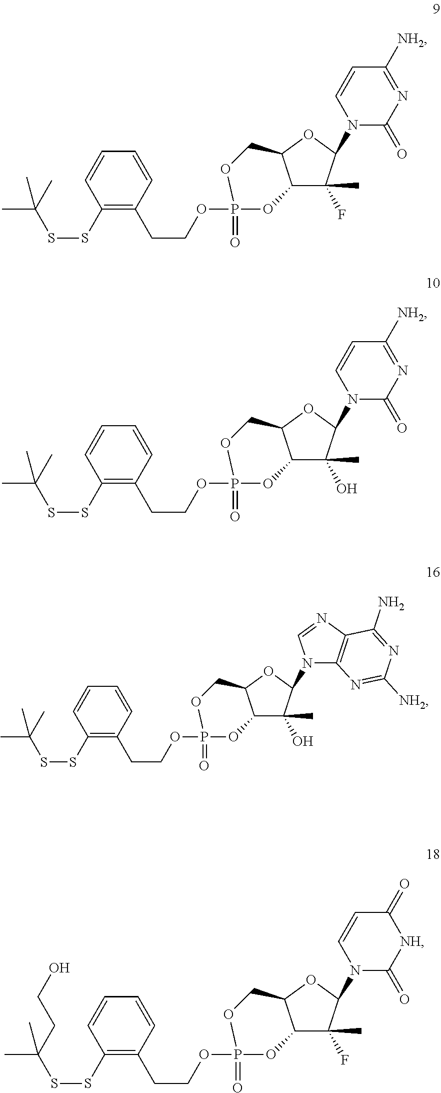 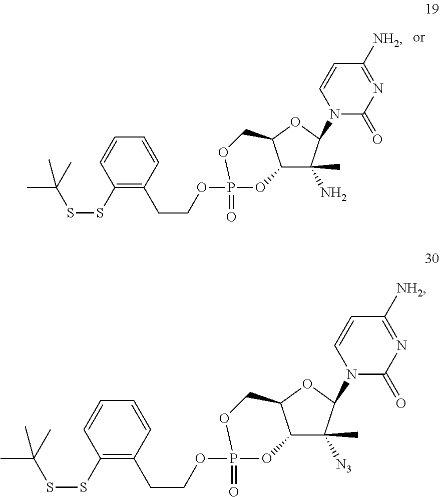
or a pharmaceutically acceptable salt or a phosphorus diastereomer thereof.

36. The mononucleotide of claim 35, wherein said mononucleotide is 4, 6, 7, 8, 9, 10, 16, or 18, or a pharmaceutically acceptable salt or a phosphorus diastereomer thereof.

37. A composition comprising the mononucleotide of claim 1, wherein said mononucleotide is isotopically enriched.

38. The composition of claim 37, wherein said mononucleotide is enriched in ¹⁵N.

39. The composition of claim 38, wherein said nucleobase comprises an exocyclic amino group.

40. The composition of claim 39, wherein said exocyclic amino group is isotopically enriched in ¹⁵N.

41. The composition of claim 40, wherein said mononucleotide is: 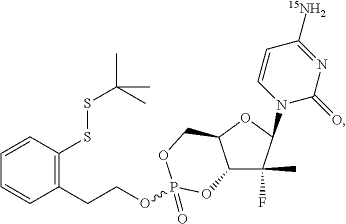
or a pharmaceutically acceptable salt or a phosphorus diastereomer thereof.

42. A pharmaceutical composition comprising the mononucleotide of claim 1.

43. A method of delivering a mononucleotide to a cell comprising contacting said cell with the mononucleotide of claim 1.

44. The method of claim 43, wherein said cell is a liver cell.

45. A method of treating a subject having Hepatitis C comprising administering to said subject the mononucleotide of claim 1.
